SEQUENCE LISTING (30 sequences)

SEQ ID NO: 1 - RNA, 78001 nt, Homo sapiens
augcugugggggaagggggaaaacucagccauguggaagcaagacaaccaauauaaaaccgucacuguguacuccccauacagagagaagguuaagagcaacaagaugggaggcagcugcauggaaccugucccacugaggaaguaaaacagguaaugggguggcagcaugagaggaguggaguguuuauggggccuauuauaugcaaguuuucuggccauauaaucaucacaucacuuuugcuagucugaacaaugcaaguuaaauguugaaggaugcaguuaaauguugaaggaugcaguuaaauuuccuagguccuugcauucugaaauugguauuuaccugaaaaauaaaccuauacaacauacagcaagacacggcaaucauauccagauuaaaacucuucaagaaugaaaaaccaaguauuaacacucaugaaugaguauuuagugcacugugcuauguaugaaucaaaaaucuuuuugaagugagagaaauuauccaggucuucguccuacucagaagggcaauacuuugugaaaaauuuagcaguaagaucuaacuuuagcuucaucccauuuuuguuuguucuaaugucuugugggucacuuagaaucuuguagggaaguaaauuauggauuauuaauuuaaaaacaugauaacaagggaaggaacuuuugguccucccagcauucauuauuuagugcaauagacugaauguguuuauauccuugcaaaauucauguguugaaaccugauuccugaugugcugauauuugcaaguggggucuuugggacgugauuaggucaugugggcagaaccuucaugaauggaauuagugcuuuuaugaaagggaccuuagagagcucucuugcccuucuucuaccacgugaagacacagucagaagacagucauccaugaaucaaaaagcaggcccccaccaggcaccaaucugcagaggcuugauuuuggaccccucagccuccagcugugagaaauacauuucuguuguuuauaagccaucuagucugugguauuuuuguuacagcauccugaacagaauaagacauucagugaacuguagugguggaguauacauuaauagcuuuuagaaauugggguagccacugaagaaugcauuuuuagaguggccacugaagaaucuacuucugauuuuucuaauuuuuccacauguguuuuugaaguuaagcagguuccuacuugauacuuuugaagaauuuagguuucaucaagaguccagagugcucuaaagauucuuaauuucaaugaagcacuuuuuaugaguuaucuguuuagcuuuucuucagauuguaaaguaaacuuguucuaaagaaaacuggaagccugaaaauuaggccaaggggcacaugcucuucuaaauauuagcagaaguuggaguagcaaugauuuucuuuguccuugaaccaaaccguuuacauuucagacaguccauacuacuucaauucuuauaaucaucauuuuuaguuuuauugauuuauuuguuauuuggagacauuuaugauguacuaugauguuugcucauaagguucuaaaguuuauaccuuugauuuguuuuuauaugcugccuugacuucuaaaggaauccaaaauaaaacguuugcugggaauuauaaagaauuucauugagaaaguaaaaaagaaaaaucuaugcaaaggaagugauucuguuguuuaaugccaucaccugucauucugucucuuaccauggcuggcuuggcuaguggcaccacgaacagaucccuacagaccccaacaccaagaaaaguagguuuuucuucuuucuuugcaguucuuugguauagauugauguuacuagggugguggaaggccaucgugugagaaaagauuacacuuaguauugaggaauguggugaggaugacuuccuuuuucuuacccuaauaauggacuuuuguuuuuagagaugcuguccuguaaccucagguuuaagaugaggauauuucuggauccaguguuuguacaguacuauuaauuauuuauuucuucguguaaaaaauaaauugauuauauucuugugagcuuagacaacagaaggcugucccaaguaggaccauucccuauaugcuggaagaaaaaaaaaaaauugagcugaccaagcugagaaccugaguuggaucauagugugguuaccagguagacuuccccaacagauauuccaaaggccaguuaaucuugauacuaauucucaguaccagggacaaauuucugcaucaaagacccugaggaaauacuuggcgacauuuucuuagguguguaucuuuaaaaagcauuaaagaugaguuucauaucuuuggucauuaccaccaacauucuuauaauaagucuguugaaagcucuuucccuucucaguuauuugcaggaaggucuuuaugggcuccugaaauuuuuucuucucauuaaugauuuagauuaugggucuuuuuguuucagauuucccuuucaggccucugaauaguuccugacuggggagacugcaugacaucacccuucugagcuucucuaaggcauugauaugguuguucuccaggucuuaagcacugcuggggcaaaacuaggguuuuuugguuugguuugguuucuugauuuucuuaagucauucagaccaaagcuuauaaaaaaaucuuguaaaacuuugcuuuucaaauccgcuuaucucaaugauaacuccuuuuucauagaaauguuaucacaugcuaucauuucaaucagcagaacacuugaaucauuacacuguuucagaauuaaaguuaagaguuaaaaauauuguaaugauuggccaggcgcgguggcucacgccuguuaaucccagcacauugggaggccgaggcaggcggaucaccugagguugggaguucaagaccagccugaccaacauggagaaaccccgucucuacuaaaaauacaaaauuagcugggugugguugcacaugccuguaaucccagcuacuugggaggaugaggcagaagaaucgcuugaacccaggagguggagguugcggugagccgagauugcaccauugcacuccagccugggcaacaaaagcaaaacuccgucucaaaaaauauauauauauauuuuguaaugauugauagaauguguguauaaauauauauauaaugauugauagaauguguguauacauaucucacacacauuuauauuauauacacauaaucuugguuuugaaagcucugugacuuauucaguccuuucaaaaaggaggaaaaaaaaaauccuuguaauuugcuggccaaaaaauaaauacuuggcaaaggcugagcaggagcaaugaagugacaucccagaugaaaggcaacagaaacugaggaaauggcgcugcaaacgcaugugguauugaauggguucauaacacagcucugagcugccguuuuucuaugggcauaauggauuagcugagccgagaauacauugaguuuucuccucgugucuaugggaacuauuucaggugaagaacuguauccugcgacacaucuuuuuagcuugaggcuaaaaucuaaauuuuaccauaaaagcacuuuucacauuucuaugccuaaggacaauuuuguuggaaucaaugcacaaaucuccuuugagaaugaugagugcucaagacaaucucuucauuucucucuuuuaaugcugacugauaggcaggauaucccuucugcuuucucccuuuuucucucucacacacauaaauguuaugugucuaucaaucagucucgugguuggcugacauugcacuaccccuauauaucacuaccagguuuuuggugaccauugcuuaggccuuuucgucauguuuucaaguaaauuaacagaauuuugaacguggcuggggagaaggaggcucagacauuuucgucccaccagaaaugaguagcugauggauaguugcuggcugaacaaauguuccagcucaugaagucccuggcuggggcugaguuuacuuucugaguaucuucacucugccuguaggugaaauccugguuucucuuacuuuggaugcaauagcuauacuaaacaaggcugaagacagaucuucauagucaugaguuuggaaaacaugggauuuguauugaggaggagguggaagugaaugggaaacucuggcuauauacaucacuaagucuaucgcauuugcaugcuggcuuugcaaugugucucauuauuuguaggagucacagugacauuaugaaauauauugauagugauggccucccaauggguuucacaccuucuacucuuuuccaacucuaguccauucccauuggauuaauuuuuuaaaaacacagcugcuauccauccacugaucccuuacgcaacaucuugcauugccugcugaauuccaaacaaaguucuugccuauccacauaaggcaggccuggcuggggcuucaacacaauuuuucauccucuucuccuauaccuuucugacuucuagaaaaacuagacuauucccuauuuccgggagcauagccuaaacucucccacccuucagacacuacucauguuguccccucuuccuucucauccuugggaauuuauguuuuuacuuuaaaaucuuuucacaucugacucaauugguaucuucuucauaaaguauuuccugggaucuucaacaucugauguacaccucacuuaaacuauuacagcauguucuauguuaauuaagauauuuguuccucagccuacuaccagacuguaaacuccaugaucaugcaugcccugaugauccuguucaaaacuauuuuccaacaucuacagaagugccucacacuuaagaggaaaauacaauaaguauuuguugaauaaaugaguaagacgaacaaaggcguuagauuuaguuccuuaaaaucuuucuuucuaucucuugcagcaucaaggugagugauugcauagaagacuggauaaauauuuguuaaauuuagaagacccuucaaaauuuuguugaauuaggaaaaacuauguuuaugaaaaaaaaauauauacguauauauagagagagauagagucucacuccaucacccaggcuggagugcaguggcacaaucucaguucacugcaaccucugcuuccagguucaagcgauucuugugccucacccuccagagcagcugggauuacaggcacaugccaccacgccuugcuaauuuuuauauuuuuaguagagaugggguuuugccauguuggccaaacugaucucgaacuccuggccucaagugaucugcccaccucagccucccaaaaugccgggauuacaggcaaggcccaccaugccaggccauccuauuacguauuguaaaacaccuuuaaauaguuaccuuucuucagacuuaaugccuuaaguugggcaaaucuggguuuuuuuuuuuucuucucauguucagauugaguuauuaaggcauacacagaaauggaaauaguagcccugggauaggacugcugguuuucugaugcuugucaauaacaacagaugccugaguaacccauccccagauaaucuccauuucaaaggggcagaggaaggaggaacuggcaggaguuauucuccuaggccugugaucugggcuuuccagcuggagaacugguggcacaugccacuguugguccgccagagccacaauauacagauuggcuuaaagaccauuaucuucacacccggugagauucucccacacccuagcuggaacuaugagauaggucguuuuagaggcuuuuuucaacauucugauggcuuauucauuugcauuagguuucagaaaugaggaauagaugacagaucaacaauuuuaguauccaugccucucuaccuaaccucugacauuuccgaccuaaacaucuaaggucaaaaguagacgagguggguggaucacuugaggucaggaguucgagaccaggcuggccaacauggugaaaccccaucucuacuaaaaauacaaaaaauuagccaggcaugguggcauaugccuguaaucccacauacuugggaggcugaggcaggagaauugccugaaccugggaggcagcgguugcagugagcugagauccugccauugcacuccagccugggcaacagauugagacucugucucaaaaaaaaaaaaaaguagacucagucauucagcugacucugagcucaugucuuaaauccuaauuuuacagcuggccuuggguuuaaaaguaugaaaaaggggccgggccgguggcucacgccuguaaucccagcacuuugggaggcugaggaagguggaucaugaggucaggagauugagaccauccuggcuaacagagugaaaccccgucucuacuaaaaauacaaaaaaauuagccaggcgugguggcgggcaccuguagucccagcuacuagggaggcugaggcaggagaauggcgugaacccaggaggcagaguuugcagugagccgaguuugcgccacugcacuccagagccugggcaacagagcgagacuccaucucaaaaaaaaaaaaaaaaaaaaaaaaaaguaugaaaaagggcccuaagcaucaaaaaugggugaucauuuguaaggcaacagaguuuaaauaauuggagauguucacguuguaauguuugcaaaugaaauauucacuaagugcaauauaucuagcuuauagcaccuucuuuacuuaugauauuuuaaucucauauucuucucuggaaaacuuuaaaggacuuuuaaggcuuucuaugagagugccaauuaugcccuguuccaugugcuaagcaugauacuugguuuccuugccuucaaaauuaacuagauuaaaaaaaguuuaaauagcuuuauagaaugccaauuuuagaaauaauacaugcacaaugcuaaaaauucaggaaauaauaucaaaaagaggaaaaaugauuaucuauaaucccccucacucaucgguaaucauuuuaacuuuuugauauauaucuuuccaaacauagguagguaauugagguuuuugccacuacuuuuaacagcaaaaaccacaauuacuuuugugccaaccuaacagauuaacgaguagaauagauaggacaaagguauuuuagaauauugacaguuuugaacuauuuuugaaacacuuucauuugguaauuuguuuuauagacauuaaugagucaauaccugaaacaucacacaaugaaacccaaauucuuaaaaccauuaaacuguuuggaaggcuuauuuuuucuucugugaagcaacuugcuaucaccuuuuaaaaaauuagcauucacucuguccaauucuaagaaaaaaaaaccuuaaaaaaagauuuaaauuucucucuaggccugauggcacagcuagaauugaguuggguguaguuuuugggccaacuccagauauuuucuucuaugaauugaccuuuuuuugaggcuuaauaacaguaaaacucuaccucauaaauauugugucaagauuugacauauuccauacauaccuacuaaaaauguggcagaauauucuccuuagaaggaaucuuuuagguaacucccauagacaucaguaaaaccucaucuaaacccuaaacagaaaacgcaauagaaaauacaauuucauauguaaaggugauaugucacugcuaucaaucaguaacuucuccauaguucuuauaccugcaugauaucuaucacugaaauuuacuguuugugcuuccaucaggauuugguuagauaagucucauauuucaauaugcccugaaauaaaaauguuaucagcuuugauuuuuguuauuagaacauuauaagcuagcuguguguugcaauaaccagcuacauauuccagaaguccauuugacccuucuguuacugggacugauauucuuuuuuguuuguuuguuuguuuuguuuguuuuguuuuugagacagaguuuuacucuuguugcccaggcuggagcgcaauggugcgaucucggcucaccgcaaucucugccuccugaguucaagcgaggagcaagugaguagcugggacaacaggugugccaccaugccuggcuaauuuuguacuuuuaguagaaaugggguuucucuguuggucaggcuggucuugaacucccgaccgcccgccuuggccucccaaaguguugggauuacaggcguaagccaccgcgccuggcugggacuaauauucuaugaaggcugccagccuggggcuaagcaccuagcuuuuccaaauguugacauguuuucucuauagcccuaccugauggacuggacuucugccuggauuggaguuugaucaugccuccauauggguguuuaccaggcguaugcauugaaccugaguuugucucuucaauacaagguaaaacagagagaaaugauuuaauucauccuauagcauuaugucuucaaauauaaguucuaauaugaaauauuugaccuaaauuauuuugaucacaauuggauaacuccguuucaaccaggugacuaauuuuggcuugaaguugcauuugaaacauuaaaaagaauacccaaugccauuauuuuuuaauuggaccuuguagacaaaggcauauucaaaauuaaaaaaaaaaguacauucagaucaugcuuuuaugcaguaaguuaacugaaauucaauagguacuaugcuuacuauggucauuucuuccauguaugcgauuauuaacauaaauuauucaggugauacguucauuggccccaguggcacuaauuuucugugcauaaacucagaauugauuuauaacaucccuaaguauuaugcuuauaucugauuauuaaaaugaauacaaauuaacucuggccauuucuuguuucauaauuucauagcauccuuugaaagaggcauugaaagaguuugccaauuguuauagaauaaacaguaucuguccgugucuuacaucccauauuagugggggcaugaugguuagugggagagacugauuggggucagacugauuuccacccuguccugccucuccccagcugagaggccuuguacauauuuaacucucuaagccuugauuucuuuuuuuuuuuuuuuugagacagagucucacucugucacccaggcuggagugcaguggcgugaucuccgcucacugcaagcucugccucccagguucgcgccauucuccugucucagccucccgaguagcugggacuacaggugcccaccaccacgcccagcuaauuuuuguauuuuuaguagagacgggguuucacccuguuagccaggauggucucgaucuccugaccucgugauccgccccccucggccucccaaagugcugggauuacaggcgugagccaccgcgcccggccuaagccuugaucucuuaaucuguaaaaugaggaaaauaguuuccaccucacagcauuguuauaaaaaugaaacagaaaacaaaugggcccaggugaaauacuuaaugaagggucaggcacacugcauucccuuucuccuucgcuugagcugaggaacugucugaauuccugccuugcacauaguaguugcuuaaucaaugucuuuaaaaugaaugaggauaugauauaauacguuuucagauguaauaaguaagcugcgaacccaaagcacaguggaaucaauuucaucaacccugcucauaaccucugcugcuaccaccuuauuucaaacuaccagucucccaucugaccuuccgcugugacuucugagcuccugcagccauugccguuguagaccccugaggaucugucuuccacacagcugcaugucccgccucugcucaaaacucuccagcaacaucuucaacucaccucuuagcauaagagccacagucuuuguagaacaugcaugguuccacaucaucugccccuaccgugugucaaccaccuggcuuucuacaugccuuccucuugcucacagggacccccuugcuguuccuagaaugugccaugcacacuucccucaggaccuacccucugguuggaguguugucuucaagauaugggagggcucacuccguuuguuccccugucccccaggcuuucucugugcucugcgcuuugugcugcaggaggcugaccuccaugaacugcuucuccuggcucccuagugcuguggcucccaggagcacuggcaggaggcuggugagcaggaggaggcagggagcacugaucucccuugcaaccuggggcuguucccuccugccuggugcuuugggcaguggcugaguuccuuuacuucuggccccacugcagucaagcagacuuuuccucuuggcuacagcucuugcuaaguuccaacaccgccacucucucccucugccccuucaggacugugugugguaacagcuuccuacuguagcuaguccguggguacugucuauaccuguguaaauagcaccuuuauucaguucucgucaauuucuccuuugaaugugccaucuguuccugcugggaucuaaacugggcucagaggucaccuccagagggaagccuuccagaauaaccucuuuaaaauaauaauccucucacucauuuaugauacuuuauccuggcgguacuuaccaccaucugacauauaauauacucauuuauauuugcugauugugucucccucuacuagacuguaagcucaaaggacaucaucuuucuuuuguuuccugaugugucuccaguuccuauuacagagucuaguuuacauaaauauguacuuuaauaaauguuuguugaguggauggauucuaaaauguagaaaauuacauuggggauuucauuucuuucuaacauuuuaaaugguaagggcagugaacuaauagacacagaaacuauucugauccaugcagagcagaauuuaaggagaggaugggauauaaaaucauugaaaaccugacugucccuagguuaggcuuguaguaaaaguaacugguacaucucugacuugagcaagaacuugaagauauuuaaacauucuacucuccugacauaucugagugccuacaguagaauuagcaagugaugaauuaaaugguuucacuggaggccuuuuaaaaguacuccaucucaguauuucucacccugaguaucuaaaugguaauagguuauggcaccaacaauaccacaaaaaagauaaauuaugcagagaaaacacuaaggauaauauggccaucauuaagcuagacucucucugaacagauaauagauuuucaaguuuuuuuuucuaauuaucuagauuaucuuuuuaaauauaucacuauucaaguaauuuuauuuuauccuaaauaucaccacauuuaugaucagaaauuaguuaauaaacucuugaucuccuccugaauuuguuuauggcguuuugaguuuacauguuaaaagguagggcuaaucucaagacuucaaucacguagaucuucauuuucagaugagccuuagaacagcgguccccagccuuuucggcaccagagacuaguuccauggaagaccauuuuuucacacaugcagggaggggcagaggaugguuucaggaugaaacuuuuccaucucagaucaucaggaauuagagucucauaaagggcgugcaaccuagguccguugcaugcgcaguucacaauagggcuugcacuccuguaagaaucgaaagccagccgggcacggugacucacaccuguaaucccaguacuuugggaggcugagguggguggaucaccugaggucgagaguucaagagcagccugaccaacauggagaaaccccgucuguacuaaaaauacaaaauuagcugggcgucaugccuguaaucccagcuacuugggaugcugaggcaggagaaucgcuugaacccgggaggcggagguugcagugagcagauugugcugcgauccagccugggcaacaagagcaaaacucugucuuaaaaaaaacaaaaaaacaaaaagaaucaaaugccccugcugaucugacaggugguggagcucaggugguaaugcucgcucaccagcugcucaccuccugcuguguggcccaguuccuaacuggccacagaccgguaguggucuguggcccaggggcuaggggaccucugccuuacaggauaccaaaguugagcucuguuaauguucugcucuucauuugaaaugagagaaugaagucugauaggcccaagaacuuuuucuuucauucuaaagucaggacuaucagaaaucucccuccgguuaggaaugguccaccuccucccaucucacuuuuuaagucagccuggguuauagcaccugguuuuuuuuguuuguguauuguuuguaucuuacaaguguuaguccugguuuuuaguugggaauagcuguugaaugaucauaugucuguggaagaaaagaauaaauggacucuggagacagaaauguucaaugcacagugccauaugccugcagucauuuacaaaugcaccuauucucacacuuggcugcauguugaaaucaccugagggaguuucagaacaacacugauaccuggauccuaucccuagagaccaugauuuaauugauaugggguacaucacaagcauuggagguuuucaaagcucuuggguauucuaauauggcagagaguuggugaacugcugauuuaggccauccaaacaucacuggcauacauuugaugcagguuucacugauucuagucacaaguagcuucugcugcuuuaacucuagagacacuacaguuccucuugcauucucccacuuugguucaauaggaaaaucucugccgcuuagugauuuuccaagaaacaugagcuucugccuuucaaugaggaagauacucagaagucauguucgagcacuccgguaagucacacagcaaggaaucuuaaucccagguagggauguuuggggguguaucagaagaauggguaaucacuucugaggagaaugaucucucagccugugaaucuuuaaccacugaagcccagagaggaguguuuucauuaacuuguggccaggcauugguaagaucuggccacauaccaguuuccuuccuaguggauuauaccauuccuugucucugagagguugugaauguuccuucuuuccccccgccccacuuuuuucugcuuguuugucugcuuccuugacuauuuaugauguggagagaccaaggcccagauauuucuacagggacacagaaaaagcaaacucuuaggggaaccgcaguacuggcagugccaaggauaauacagcucuacuuaagcuagaaaucucucugcacagauaauagauuuuugaagauuuuuuuugguucuucuaauugucuagauuaucucuuuaaauauaucacuauucaaguaauuucaauuuauccaaaauguuuucaccaaauuuauuaucaggauuuaguuaauaaacucuugaucgaaggcaaaagaccaauuuugccauuuuuuuguaaaguagagggaaauaugcaagaguggcagagugggccgugaaagagaagugcagaagguuauuggucuuagaccucauuuccuuguuagauuuuuuucuaaaccugaccaaauggucuacugaaguacucacaugguuuaaaaaaucacuuuacaaaaauuaaaucaaaggggaaaaagcucgguggcuucuggaaggcauuuguacuagcauuugaggugguguuuauagcucauaccuguaaucccagcacuuugggaggcugaggucggcagaucacuugaggucaggaguucgagaccagccuggccaacacggugaaaucccauuucuacuaaaaaauacaaaaaucagccaggcgugguggcacacaucugugaucccagcuacucaggaggcugaggcgggagaaucguuugaacccaggaggcagagguugcagugagccgagauugcgccacugcacuccagccuaggugacagagcgaaacuccaucucaaaaauaaauaaauaaauaaauaaauaaauaaauaaauaaauaaauaaagugguguuuacaaagggaucauaacaaucaguucaucagaauuuggagggcaaaauugauucuuauugacaauuuccuggaugauagugcaauaugaauuaguuacuauaaaagcacuuaucuccuuuaauuuaauuugcugucucagauuguccaucaggggauuuuugaggaaaaguguucaaggauauucugaacuuuugguucuguuaugaugucagacacaggagacccagucaggugucuggacucuggaagagcucucuaugcugagagagagaacauggaagcuacuggcggagcaaagaauccaagaaauuuauuguuuuuuaacacuggcuuuuccuccccacucuacuggaaacaacacauauguauuuaaaagaguuucacauauacuaaugcacagccaagcuucuugaguguaccaaaagauacugucuaguuuccauaaaagggcuguauuaugccuagauaauaccaagauuguggagugacaacccccaugaguagcggaguuaauauuccaauacucuacguguuuacugagguacuuguuagcucccugucuuuagggauacaccuuaacaaagcuuagaagcgcagagggccaauauuuauacuuguuagacaugccuugaugguuuggagccauuuccaacaacagcuguacaaaugguugauguuauggugcuuucucagacuuaaaucccauuuggcaauuccuauguaugugcaaacacugaaaauauuuucauuuuugacauguaggggaauucaauuugggauuuaaauguuaacccaaaagggaucauccauucuccugcuugcccuuccacauauguuccucccucguucucccucucagugacagugcugacugccaugacuuccccucaagaaauucccagagccugcucuuuugugcugucauaaacauaaggagugaaacauuggcuggaaauguaaacuuggaucgaguuuccaaaacacaacagaacaauugguggucuugucaaguccaguccuuaaauuggacaaaaccauagccuugacuauucuguuguuuauaagguucaucccauauauaaacgauuuuuuuaaaauuaugggagauacacauuuauaagugcagugaaucuuuguuuagaaaaagcaauuugucugucuccugaauugguaagcuuuuucucuuuccucuuuccagcccaaaguaaaacaaagcacuguuucaccuuguaauugcgggugagccacaaugaacuugcucaauguaaagaaaucuucauggcacuuuaaaucauuuuggggagaauauaguuguuagauaagagguguugccuugugagccacgauucuuggaaaaugucugagacuuaggcacacacaugugcacaugcaaacacuugggcaaacacauaugcagcuuccaaaguuuugucagauuuuauuagaagaggauuuucuccuuggcuccguguacacugcuuguacccugcuaguuaagggaaguggaagauccuugguuuauagggugaaaagcccaaguggcaggauaaccauccauaaaccagccucaggaaggggccaaguagggauaacugaaauuagccacuaacugaugauaauggggauuaucuggggguaacuuuggggaugcacuaagcaacauacauugggguugcuuuuuaaggaaaaaacaguguacuucugagaaaggaaaugcuuuuuggagucaggacaaauuuaggccucucuaaucuaaaaagucccuuugccugauuuuacggccauagugagaaaagagcaagguaaauauacaaggccacccacagggacacagagccuuuccccugccuguguucucacuguaaaaauaugcauggccacucacacacaugcauacacacagacacacacacaugcauucaugcacacacacagguaugcacauauacauuugcaugcacacacacacacacacaccucaaaauuauuucuugauacugcuuuguagaaacagaaacaggaacacaccauauccaggcuacauuugguagagaacauuccagcuuugugccucaguugauuuccuucucucuuccucuugcucucuuuugguaauuuauucacauauauaucaaacaggagaauaucauuuuuaaaaaugugacuaggcugggcacaguggcucaugccuauaaucccagcacuuugagaggcugagucaggcagaucacuugagcugaggcguucaagaccagccugggcuacacggugaaaccucgucucuacaaaaaaaaaaauaaaauaaauuaaaaaaucccugguuaggugguacacaccuguauucccagcuacucaggaggcugagguaggaggauuccuuaaaccugggaaauugaggcuguggugagccgugaucacaccacugccugccuggauggaagagugagaccugucucaaaaaaauaaaaaaaaaugugacuaacaauaauauggauauuuaacuugcccagugcgacuuauacauauggccacagauacagaaccaggcauuagagaauuaucuguauguuuugaacauuuuacaggaaaauguccuuggaguuucaacauuucuuuggucuuccacauuucauuuuguccugauuaaagaggaagccaguugcuguuugugguaggugcucccugacgugggugccaguggaaucucaucuucuggaauuuauauacuuguguaaucacauccccuggaguguguggauuggaugcaguggugugucucuaauaaaugaaauuuggaaaaagugguaagaugucacuuccaagguuagauuaugaaagaugacuacuuccuucuugguuucauccucucucucgcccucucacuugcucacucugaugaagcaagaugccgugcuggaaccugcccuguggagaggcauacauggugaaaaaccagggaaggccucugagcaacagcucuugagaaacuaaagcccuaaacccaucaacuugcaaagaacugcauccugccaccaacugcgugagugaggucagguggaucuuucccugguccagccuugagaugacugcagucccagacaacaccuugacugcugccuugugaugcagcuggcgccaagcagugccaauuccuggcccucaaaaaccaugagauaauagugcuguuguuguaagucacccaaguuugggguaauuuguuaucuagcaguagauagccaauacaucaaacuagaaacauucuccaucauugauuggaagacuuuggguuuuuaaaaaggguaaacacacauauacacagcuuauuauauuuccuuccacauuuauuuuggaggggaaauguuuuacuucuuaucccaccgcuuugcccuuucuaaaagaauguguuaggauuacugugauucaaaugguaaaaauuuucccagugaaauaaagacuuuauaccaacuggauuucuugcuuagaaagcuguguuuucuccacuaauaucuugaauuugauauuuucaacuaucucuauugaauucacuuguaaaugaauugcguuuuucccucuauuaugaacugaauguuuguaucucuucaaaauucauauguugaggcuguaauucccaguguggcuauauuuaaagaugaggccucuaaggaaguaauucucguuaaauuuggccauaagauucccugaucccaucuuggcuaacacggugaaaccccaucucuacuaaaaauacaaaaaauuagccgggcgugguagcagguaccuguagucccagcuacucgggaggcugaggcaggagaauggugugaacccgggagguggagcuggcauugagccgagaucgcaccacugcacuccagccugggagacagagcgagacuccaucucaaaaaaaaagaagaaagaaagaaagaaagaaagaagauucccugaucccauaguauuaguguccuccuaagaagaggaaacaggggagcucacucuuucucuccuucucucccaguggccaugugagcaggcagggagaugguggcugccuagaagccaagagaaguggccucaagaugaaaucuaccuugcugguacuuugaucuuggacuugcagccuccagaacugugagguaaauuucuauuaagugugugggauuuuguuacagcagccuaaguugacuucuguacccucucagucugguuuuguuugaucacuucagcaacaaaauaauggagaagacaaaugcaguuauuuggaggaaagauuaaaacuuuccuuauacaaaauuaaucccauaaguauuuauuaggcaguuauccauguuugaaauuauacuagacacuuuaggaaauaguuugguauuuacagauuaucucuacuuggaauggucugguaaaaauagguggguuuuagaauuuaauuaguuugaaugacucagccacauauucuuguccacuaccacccacugaaaaaauugucuccagauguucaacaaggcugagugaucaauaaaacuggcaauuugaccagcagugaucugggacuuaagggaauguaccuaugaucacauuuuggacuuucugccccaauauuuccaacaaaaaugacgcauauuucaccaccugaagaaugguaagugaauucguggugcccaauuugguguuauaaaacccauucuuacaacacgaaaaaauuguuaugcauaaaacuugugaugagaaaccaacaugugggaaaucuaguuuggugaaucacaguuuuucuauacauggguuuagaaaccaccgaguaucucauagucuauuuuucaauaagaauggggcaaaaugguaguauuuuaucuuccagaaaaaacaaaaauuaaagaaaaacaaaucaaaccuuaaaacuucucugucaaagauaaauguacauucuucugaauucccugccccaucacaaggcuuuuaggcagcaucuauaccaagauagcaauuaugccaacccugaacauguagguuaaacauaauccuuauauuuucuaccuuaacuuaaaugcuugaaggguacacaauaauauaugaugauaaauuugaaaaggaugauuuuaauuauaaugaagaauuuuuaacaguugggaguuggcugccauuuugaaagaugagauaauuauugauaaugugucugaggcuaagacagagcaagcugggucccauuuuauugauuuuuuuuucuaaaaugauuaaagugauauaucagaaaauacauuuaauuuauaaucccugcaaugaaaaaauacucuuauauuauuuucaacaugaagagcucugacauucgagcuguauuaggccuuaaaagaacuaucucaucgggugaucccacacucauaugauugcugcucugcaaacucaaucauuagaccucaguuuaaucuuaggguucuguucaaggagaaugaacugugcccauaugguaugguuuucuuccaaaggagauugagaagagauugugguuucuugcauuaaaggccauuuggcuucauaagacuuauuguugauacucccuuccucagagcacagauguauuucuguuucugacuagugacuucugucaggucugaaauguuaguuuuauagaaauccuggcuguguaagaagaauggggaagguggauugauuccuagucauugugaacagacauuccucuucucaugguaccacacuuuggacaggucagcuacccagagaaagcucuacaaccccaacaccaaccuggacuaggucccacaagaaagccugacaugucaggugcugggacacucaucucuggcuagucacugagcccagcagcuccagaugaccuggcgccaccacuuccugacagcuucuucuaggaucuuacauucaguauauccggugugacuagucagaagucacagaagccuacucagaaguaaagcuagugauaagaguuuaaaucuguguuccugcccaaaucucauauuaaaauguaauccccauuguuggagguggggccugguuggaggugauuggaucaugggggcagauuucucaugaaugguuuagcaccauuccccuuggugcugucgucacgauagugaaugaauucucauuugaccugguccuuuaaaaguuugcagcaccucucccaacacuccuccucuugcuccagccaugugaagugccuguccccucuuugccuuccaucaugauuguaaguuuccugaggccuccccagaaggugagcagaugccagcauuaugcuuccuauauagccugaagaaccaugagccaauuagaccucuuuucuuuauaaauuacccaaucucagguauuucuuuauagcaaugugagaagagacuaguacagcaagugagucuuacaaaugaauauguuugcccuuuaaagauaaucaacuuuuaagaacugaauacuauuaacuguguuagcucuuggcugcucuuagguugggaaugucauacaagguagggaaaguauguuuugguggguaagagaaaagggaacacuuucauacuauuggugggaauguaaacuaauauaaccacuauggaaaaaaaguguggagauuccuuaaagaacuaaaaguagaucuaccauuugauccagcaaucccacuacuagguaucuacucaguggaaaagaagucaugauaugaaaaagauacuugcacaugcauauuuauagcagcauaaucugcaauugcaaaaauauggaaccagcccaaaugcccaucaaucaaggaaaggacaaagaaaaugugguauaaauauaccacaaaauacuauucagccauaaaagaaacaaaauaauggcacucugcagcaacguggaugguacuggagccuauuauucuaagugaaguaacucaggaauggaaagccaaacaugguguauucucacucauaugugggagcuaagcuaugaggaugcaaaggcauaagaaugauacaaugaacuuuggagacuugagggaaagguaggggguggugagggauaaaagacuacacauuggguacugcuugggugaugggugcaccaaaaucucagaaaucaccacuaaagaacuuauucauguaacccaucaccaccuguucccaaaaaacaaaauaaaauaaguaaaauauuuuaaaaauuuaaaaagagugaaauuucuggaaucagauagcuugggaaaguccauuaaucuuucugugucucaguuuucuuaucucuaaaaccaaaauaaaaaacaguacuagaaucauagaauaauaaagcuuaaaugaucauacuacagagcuaggcuuaaaugagcauacuacagagcauacuacacuuacuacaguaaguguucaauauauuaguuuuuugcuauugaauaaaaauuacaacuuuccuuggcuuacaggaauugaauuuauuuuacuaaguauauauuacuuuauauauauauauauauaauauaguauauauauuauauauacuaaguagagugcuucucaaacucucugcauuaaaaggccagguuuuuuguuugcaauccauuguagccugauaguuuugcaaauuauaaucaaaacgaauuaguagaaaaaugaaauaaaaugaaacaaagauauacaaaaccuaagcuuaaacagcuguaagaagaguuucuaaaugcuuccucucaauuucuguacuucugucuuugucacuggguugcauaguucccaggugggcaccuguucacagaguagcacuggaagaaaaaaagcagcuguguuucugcuggacuguggugcacgugcagcuucuccuguguuggggacuccccuccuccugagguggagcucagcucugagggcuagugcuaaaaacgucuggugcuugcaaucucacuguucuuauagcuauggccagcucagggcgguggaccuggacccaucagacacacucauacuuagaaugcaagcuaaacaugagaagaagcaggaagaacacaggagcaaguccaguaaaugguagaggcagcaccagcagcacccucuacuuccagagguaauaguagccgugauuuuagaagcaucauggaaggcagcugugaaugguccuucaccagaccagcauccugacaugguuguggucugcuguccucaagguggagucucucugguuuucagauucuucuggagggauagugauugacccaacacucuuuuaacaaguuuuuuuuuucugcuuaaauuagucgauagauuuguauugcuugaaaacucagaaccaggauuaauucauauggauuuagaaaagaaaaccaugaagauaggugauccuggcauuuggcaucucaucacagcuggggauccuauagauguuuagaaugcuuugguauugaucucuguauuaucaucucauguacucacucuagcucuacuccuuuuuggucuuguucacaguacaugcuaacaauacccuucaguuuagaucugaaaucgagcugaagauggaugaauuugauacuaccuuaugacagguuuaagugucaugggcuuguaggcaagccaucugucacaaggaugaaaggaacugaucuucccagaguuuccuuuuuuagccugggggaauuuauaccaagaauucacucuucugugauacuaagcaaagauuuuguucccuaaagcugauuucucucauaggaaacugcucaugugaaagccuccugccauuuaaugggcagcaaagguaauguuuugcuugguggcuaaaggccucuuuaaccuccagagaggcuaaguauggugcagacuccccuagggacacguguucuucauuccugguggagggcaagcagaggcuauccuggcgucugguagggaugcuuuagaauuagaaagaaggagauuaaaccugaagauuucuuuucccuucacaacugagaaucagguuacugucuuuaaggauuucuucuggccguuauaaaugcugggcuuccaguuuaaucggcuagcauacuagcauauuuugcaugacuucugcaaggagguaaaaggauaaaaaaagaugaacuucuagaagugaaaauuaaugcacuagcuuucaucaucaagacagaaauaauucuugagaaagaaaacaagugaaauuguuacccagaaaacuauccaagucuggucccauuccuagagcaacugaugcaacugaucuagauggaccuaaacaucaagauuuuuaaaaagcuccccagcuuucugauacuaaugcaaaaugacagaaccauugcuuucugguuucugguggagacucuggguucugcuuucccuucuuaggggagauguagccagaguucaggucagccaagggcagcuugaaaagugagaaggaggcuaggaaagaauauaugaagacccuaagugcauuuaaucuccuccaaaauguaguguauggacagaaaacucagccuggcugcgucuccauuuucucuagcuuccaucacauucuuuaauugacuguaugagcugugcuuuaguauggaugaaaggcagugugguuuaauaaaaagggucaccauuagaaaggguuccagauuuucuguaggaaauacauguguccaaacauuagagaacuccucaaggaguuggagauccagaaacuagaucauaauuugagaauuaauuauaaauaguuugaagauaaguuccaggcucuuucuccugggcuaugaagauuaaaucgagagcccacacugguccaucuucaugcagaguugcuggagaacauggagagagacuuggggggaaagaagccaucaguggguauuugguuaacagcucagaaaggcauuauuuguagggcaucacuuugaguuuauuguguguuucaguauguauaaguaucaggaguuuauugacagaaauauugacaucucccuaaacagcuuccauuauagcuacuguagaaguaacuucaguccucuauaaucccugacuccccgcaacuucugcaguguucuuuguucaaaaagauguggcugaccuugaggguggcgugcucugugaagugccugccagaacaacugaaggauuuacauagccuuuucacacucacguuguccucauagugcagcacuuuggggagcucauuggucuggguuuuggucugcauuuguagcuaagcuacacucccagagggcugaaacccagcaccucgccccuuuaggacuaggccugaaagucugguaaggcuagaaggucaugcgagaaacuguaugaagagacacagaguaaggaugggcaugagagaugaacacgaacaaguguggaagaucagaauuuuuucagaggguaacugguuauaagugcuauguuucucuuuagcgggaauauuaaaaauauaguaauuucuuuagcaaacaggugaucagacccgcaaaaauuuagaaagcauuaugaguuauuuuaaaaagagaaacucaguaccacagauaggcuuggcaaugcagaaauaacaguagacuaaaaauuaauauuacuaauuuucucuuugcuuuauuaauuuccucuauuacagaaucugucuuuugcuaccuacagagagauggcuccacauaaucauuuuacauuauuuuauuauucauuguuauuuaccuugccaagugcauagaucuacacagcaaaaaauuagaaugacaaagauaggcuguuuguaucaucccaacugcaucucuucagucucuauggaauugaaauacgauuaaaugaaucccucaugaaaauuaacaggcaacaagaaggaaauuauuauuucuguugccauuucacauuagucuaacuuuucuuugaauauugcauuauaaagucaauagaaacuaugcaaagugacugagcaagagcuuuccauucaaauucucaaauaggcucauucauuuucauaacccaaauauaaucauggaauaaguaggaggacuucuggagacuugaaacaauuuuuacauguuacugaagagaaaacugcugcaaaugucaagaguugcagauauguugugaagccagcgacagcuuuucaggaaaaaugggaugcugugugcuggggguuugauuugucuuugguaguaacuagcuaugugaucagauacaagccagcugaccuuucugaauuuggaauaaacgcugauaucuagaaaaagcaaauucacagugauguuaaauauuuaaaaucucugcucuuugacauacaaaacuuuagaggaaugaacccagguuuagaaugccuuuucaguucuucauucaucaauucacaaaauacuuaguaaccaucucccacuaguagggcucagugccucacacugauaaauuuguuucauucugcaaacuaucaggcaucauguagugaaaauacuuucuaaaaggcauuaccugacuuuaguuuucuaauugcuuauuuuaaagaaauugaaauaagagcucccauaaguggaacuuagaugacaaauauaccuaacuauaacacagaaagguuaaaaaauagauacagguuaauaguuucuuacuauaagaaguaaagucaguuucuuauuuugauguccaugugcaguaacugguagaguuucuaaguuucacguuucuaauacucaaaugaagagaaaccuuugaacauggagcuuaaaagcaaugcccuuacugagugcaaaaucaacacauuagcuauaaauuaaucaacuaaaaugucauauuuucaaacuaacuuuaaaugcaacauaauaaguaucuaguuuaaaauaaugggaacaccuuuuuuuuuuaauuauuauacuuuaaguucugggacacauguguagaacguucagguuucuuacauagauauacaugugccauggugguuugcugcacccaucaacccgucaucuacauuagguauuucuccuaaugcuaucccuccccuaaccccccacccucugacaggcacccaugugcgauguucccuucccuguauccauguguucucauuguucaacucccacuuaugagugagaacaugcgguauuggguuuucuguucuguuaguuugcugagaaugaugguuuccagcuucauccaugucccugcaaaggacaugaacucauucuguuuuauggcugcauauuauuccaugguguauauuugccacauuuucuuuauccagucuaucacugaugggcaucuggguugguuccaagucuuugauauugugaauagugcuuuaauaaacauacgugugcaugugucuuuauaguagaaugauuuauaauccuuuggguauauacccaguaaugggauugcugggucaaaugguauuucuaguucuagauccuuaagaaauugccacacugucuuccacaaugguugaacuaauuuacacucccaccgucaguguaaaagcuuucuuauuucuccacauccucuccaggaucuguuguuuccugacuuuuuaaugaucaccauuuuaacuggugugagaugguauuucauugugguuuugauuugcauuucucuaauggccagcgacgaugagcuuuuuuucauguuugcuggccguauaaaugucuucuuuugaaaagugucuguucauauacucugcccaauuuuugaugggguuuuuuguuuuuuucuuguaaauuugcuuaaguuccuuguagauucuggguauuagcucuuugucagauggauggauugcaaaaauuuucucccauucuguagguugccuguucacucugaugauaguuucuuuuccugugcagaagcuauuuaguuuaauuagauccuauuugucaauuuuggcuuuuguugccauugcuuuugguguuuuagucaugaagucuuugcacaugcaaugguauugccuagguuuucuucuaggguuuuuaugguuuuagaucuuauguuuacaucuuuaaucuaucuggaguuaauuuuugcguaagauguaaggaagggguccagugucaacuuucugccuauggcuagccaauuuuccuagcaccauuuauuaaauagggaauccuuuccccacugcuuguuuuaucagguuugucaaagaucagauguguguagaugugugacguuauuucugaggccucuguucuguuccauuggucuauauaucuguuuugguaccaguacuaugcuguuuugguuauuuuagccuuguaguauaguuugaagucagguagcgugaugccuccagcuuuguucuuuuugcuuaggauuuucuuggcuauaugggcucuuuuuucauuccauaugaaauuuaaaguagguuuuucuaauucugugaagaaagucaaugguagcuugauggggauagcauugaaucuauaaauuacuuugggcaguauggccauuuucacaauauuuauuuuuccuauccaugagcauggaauguucuuccauuuguuuguguccucucugauuuccuugagcagugguuuguaguucuccuugaagagguccuucacaucccuuguaaguuguauuccuagguauuuuauucucuuuguagcaauugugaaugggaguuugcucaugauuuggcuguuugucuauuauugguguauaggaaugguugugauuuuugcacauugauuuuguauccugagacuuagcugaaguugcuuaucagcuuaaggaguuucugggcugagaugaugggguucucuaaauauaaaaucaugucaucugcaaacagagauaaauugacuucaucucuuccuauuugaauaugcuuuauuucuuucucuugccugacugcccugaccagaacuuccaauacuauguugaauaggaguggugagagagggcauccuuguccugugcugguuuucaaagggaaugcuuccagcuuuuguccauucaguauauuggcugugggucugccauauacagcucuuauuauuuugagacauguuucaucaauaccuaguuuauuguguguuuuuagcaugaagggcuguugaauuuugucaaaggcuuuuucugcaucuuuugagauaaucaugugguuuuugucguugguucuguuuaugugauggauuacguuuauugauuugcauauguugaaccagcuuugcaucccagggaugaagcugacuugauugugguggauaagcuuuuuaaugugcuacuggauuugguuugccaguauuuuauuggguauuuucgcauugaucuucaucagggauauuggccugaaauuuucuuuuuuugugugugucucugccagguuuuguuauuaggaugauuuuggccucauaaaaaaugaguuagggaggauucccucuuuuucuaucguuuggaauaguuucagaaggaaugguaccagcuccucuuuguaccucugguagaauucagcugugaauacaccugguccugggccucuuuugguugguaggcuauuaauugcugccucaauuucagaacuuguuauuggucuagucagggauuugacuucuuccugguuuagucuugggagggcguauguguccaggaauuuaugcacuucuucuagauuuucuaguauauuuguguagagguguuuaugguauucucugagaguacuuuguauuucugcgggaucagugaugaucuccccuuuaucauucuuuuauuguguauauuugauucuucucuauuuucuucuuuauuagucuggcuaguggucuaucuauuuugcuauucuuuucaaaaaaccaccuccuggauucauugauuuuuuugaaggguuuuuuugugucucuaucucccucaguucugcucuaaucuuuguuauuucuugucuucugcuggcuuuugaauuuguuugcucuugcuucucuaguucuuuuaauugugauguuagggugucaauuuuagaucuuucccgcuuucuccugggggcauuuagugcuauaaauuucccucuaaacacugcuuuagcugugucccacagauucugguacauugugucuuuguucucauugguuucaaacaacuuauucauuuuugccuuaagucauucaggagcagguuguucaguuuccauguaguugugcaguuuugagugaguuucuuaauccugaguucuaauuugauugcacuguggacugacagacucuuucuuaugauuuccauucuuuugcauuugccgaggaguguuuuacuuccaauuaugugguugauuuuagaauaagugcuauguggugcugagaagaauguauauucuguugauuugggguggagaguucucuagaugucuauuagguccacuugguccagagcugaguucaaguccugaauauccuucuuaauuuucugucuuguugauaugucuaauauugauagugggguguuaaggucgcucacuauuauugugugggaaucuacaucucuauguagucucuaugaacuugcuuuaugaaucugggugcuccuguauugggugcaaauauauuuaggauaguuaacccuucuuguugcagugaucccuuuaccauuauguaauguccuucugucuuuuuugaucuuuguugguuuaaagucuguuuuaucagagacuaggauugcaaccccuguuuuuuuucuugcuuuacauuugcuugguaaaucuuccuccaucugcuuauuuugagccuaugugugucuuugcacaugagaugggucuccugaauacagcacagcgaugggccuagacucucuauccaauuugccagucugugccuuuuaauuggggaauuuaguccauuuacauuuaagguuaauauuguuauuguuauuguuauuguuaugugugaacuugauccugucauuaugaugcuagcuggcuaguugaugcagcuucuucauaguguugauggucuuuacaaguugguauguuuuugcaauggcugguaccaguuuuuccuuucuguauuuagugcuuccugcucuuguaaggcaggucugguggugacaaaaucccucaguauuuccuugucuggaaaggauuuuauuucuccuuugcuuaugaagcuguuuggcuggauaugaaauucuggguugaaaauucuuuuaagaauguugaauauugucccccacugucuuguggcuuguaggguuucugcagagagauccgcucuuagucugcuggacuucccuuuguggguaacccgaccuuucucucuggcugcccuuaacauuuuuuccuucauuucaacuuuggggaaucugaugauugugugucuuggaguugcuauucuugaggauuaucuuugugguguucucuguauuucauaaauuugaauguuugccuaucuugcuaugguggauaaguucuccugaauaauauccugaaguguguuuuccaacuugguuccauucuccccaucacuuucagguacaucaaucaaacguugguuuuuucacauagucccauauuuccuggaggcuuuuuccuuccuuuucauucuuuuuucucuaaucuugucuucaugcuuuaaugaucuucaaucucugauauccuuucuuccacuugaucaauucagcuauugauacuuguguaugcuccacgaaguucucgugcuguguuuuucagcuccaucaggucauuuaaguucuucuguaaacuaguuauucuaguuagcaauuccucucaccuuuuaucaagguucuuagcuuccuugcauuggguuagaacaugcuccuuuagcucagaggaguuuguuauuacccaccuucugaagccuacuucugucaauucgucaaacucauucuccauccacuuuuguucccuugcuggcaaggaguugugaucuuuuggaggagaagaggcauucuaguuuuuggaauuuucagccuuuuugugcugguuuuuccucaucuucguggauuuaucuaccuuuggucuuugguguuggugaccuuuggauggagcuuuugcauugucauccuuuuuguugauguugaugcuauugcuuuuuguuuguuaguuuuccuucuaacagucugcaggucugcuggaguuugcuggggguccacuccagacccuguuugccugggugucaccagcagaggcugcagaacagcagauauugcugccugcuccuuccuguggaagcuucaucccagagggacaccugccagaugccagcaagagcucuccuguaugagguguuugucagcuccugcugggagaugucuccccaucaggaggcacgugggucagggacccacuugaggaggcagucugucccuuagcagagcuugaacgcugugcugggagaucugcugcucucuucagagcuggcaggaaggaacguguaagucugcugaagcugugcccacagccgccccuuccccugggugcuugccugagugagaugggaguuuuaucuauaagccccugaguggggcugcugccuuucuuucagagaugcccugcucagagaggaggaaucuagagaggcauucuggcuauaguggcucuguggcacuguggugggcuccacccaguccaaacuuagugggaacacuuuaauauaacuucuuuaauuuacaagugauauaacuacauaauaaaaaugugauagaaaaacuaauuuauagucaauauacccagcaaaaccagcaugaacauguuugaguaauccccccaccaucuuuuucuuacuaaauuucccucuacccuaacugacacuccaaaaaacccuuaaucuuuuccuuccuuccagguguaucuaaccucauccucucuacugccacaggagggacuuuucugauccgugcauuuguuucuccauucucccucuucaaaugcuccagcagugccuuggagucuuugggauaaguccacacuccacagcauggucuuuaagccucuugauuuugucugugccaccucaccaaccucugcucuucauacauugccacacccccaucuccaugcaaccccugaaugccugcuuaugauuuuaaacucaacccuggcucaccaacuccaaaacaucauccccaucccacccccauggaucauuuagguugguaggucccuucuuuuccccccucaccauaccuugacaauagcaacuaacuuauuauuguuauuauugacagagggucucacucugucacccauccuggaguacaguggugcaaucgcggaucacuauauccuuaagcuccuggguuuaagugauccuuccaccucagccuccugcguagcugauacuacaggagugugccaccacauccagcuaauuuuuguauuuuuuguaaagacaggauuucaucauguugcccaggcuggucuggaacuccuggacucaagugaucugcacaucucagccucccaaaguauugggauuacaggugugagccaccauggccagcuuaauuauuauuuugaugauuccccaaaacaguauugaugaaaaccgugagauuacauagcaacaauauguauucccaaaacaccaauuuuacucaguggcguaguuuuaaacacuucaugacaaaauauaugcucaaaauauguuuugaagauugacuacuaaucugggcacuggggauacagcggcgaacaaaacauauaaaaaucucuauuuagggaucccuaaauccugcuuagggaucugcuauuuagagaaggauuuggauaagauaccaaaacaaauuaguaaauucuauauuauauuggaaagcauuaagagggaagaugacguaauuuauugucucagucuggacaauucuugcgugaaagaagugccccugauaauuacaccagggccacaugugucggccggaaguacggcugauaacaacauuaaagcaguguuuguuaacggggacugucccauuuagccuggggagcaugaugugguaggcagcuucugacaaggcucccccaaaugccugcuuccugguauucacacccuuguguaaucaccucuaacuaugggcuagaccuagugacuuccuucuaacaaagaaaauauaggcaaagugaugggauaucacuuccaagauuaggcuacaaagucugugacucccgucuuacuagcacucucucuugccuucuaggcuuguccauuuuaaugagaagcuuauguggcauggaacugaguucacccccaaccagcagcuggugaggaauggagcucuucguucaaugacucauaaugagcuuugagacaaccaaagcccccuagcuaaccugugccuggauuccuggcccacaaagacagugagagaguaaguacguauuguuugaagucccuuaguuuuggggugauuuguuacacagcgauaguuaacucuuauauacaauuacucuagugguaaguacuacggggaaaaaaaugaaguagggaagagaacuagaaugugaugagguugggggugagggaugaguugaaaucuuaaguagcaugaccaaggaaggacuuuaaggaggagguuccauugaauggaaacuugacagaggugagauugugucauguagacaucugcggggagauccuuccaggcugaggggcauggauuuguaaugagguggaggugaaaauucauaugugccaaccaucuucugagugcuuuaccuauaccuucacaugcuguccacacagaagccuaagcgacagguucuggauucuauccauguacagaauguggccacucagauuuaguuccccaaaucacacugcaguagcauugaugcuggguacuaaacacaguucuguugaguccaaguccaugccggagaagguaucuuaguuuccuuuugcugcuguaaugaauuaucauggacuuaguggcuuaacacaugcaaauuuauaauuuuacaguuuugugggucagaauucuggcauggaucucaccaggcuaagaucaaggcaucaggagagcugcauucuuuucuggagguucuagaggcaaacucauuuaucuucucauucagacuguugacagaauuccacuguaggacuuggcuuugcagguuguagguuccuguugccuugcuggcuugccagccaggggcugcucccugauucuagaggacacccacauuccuuggcccauggucccuuuccuccauuuuuuuuuuuuuuuuuuuuuugagacggagucuugcucugucgccacgcuggaguacaguggcgcaaucucagcucaccgcaaccuccgccucccggguucaagugauucuccugccucagccuccuaaguagcugagacuacaggugcacaccaccacacccggcuaauuuuuguauuuuuaccagagacgggguuucaccauguuggccaggauggucuugaucucuugaccuugugauccgccugccucacccucccacagugcugggauuacaggcaugagccacugcacccagcccuuuuccuccauuuuuaaagccagcaacaguggguagagucccucuaacacuuugaaucuuuccugccucuuuguugugguuguguuucuucugccugucucuuccacuuuaagcacccaggugaucagacugagcccacuuaaguaaucaagauuaaucucccucuauuaaguucaguaaccuuaauucaaucugcaaaauccuuuuugccauuuaauagaacucauucacagauuccagagauuggcauacagacacuuugggggccauuauuguacuucccagaggggcauuuugggaaaacaauaucagaauuacugaagauacaaggauguucuguuggaguugaaacacaaguaaaccagucacugcugugacaggucuaugugauagauguuauuuaugggacugacaccaacuuccuuagacugaaggacugucaagacaggcuuucuguaggacguguguuugaguuaagucuuaaggauagucaggagaagguggagaugauagguagucuucuagagagaggaagccuggagcaaaaucacuaaguugaugaacagcaugcccaguuuaaagaacuguaugcuaagaacugaaagccaccugcuuuagggaagggaagacugugagugauuccaagucugagagaacaggggacuuuagugcauagugaguacugaaacuuuauccuaaaggauauggauuugaauuuacaaggucuucuaauuaaagagauuugaauaucaggauuuauauguagaguggaaaaaaagcgcugcucuaugccucaccuccaaauuuauuuccuaccauaauguaucuuucuuaaguuucuaacuuaacucucaaguuagacagggacaaaaguugguucauucuaccccauuccauauaagugaugggcauuaauagacuggguuaaaaaaaugugguacauauacaccauggaagacuauacagccauaaaaaggaaugaaauugugucuuuugcagggauauggaugaagcuggaagccaucauccucaguaaacuaacacaguaacagaaaaccaaauaccgcauauucucacuuauaagugggaguugaacaaugagaacacauggacacagagggggaacaacacacgccaggucuuguugggggguggggggugaggggagggaaauuaaaggaugggucaauaggugcggcaaaccaccauggcacacauaugccuauguaacaaaccugcacauucugcacauguaucccauuuuuuuuuagaagaaauaaaaaaaaaguaaugggcauuagcuacagauaaaguuaaaggaaaaaauacaggaacaauuucucagaaguaucauuugaaaacuucugaucaugaguggcuagcuaucugcacauauguguuauauuacuguauucacaaccugugaauguuuuggugguuuucucugauaaugcacauuauauauuagacauugaugucagugacuuaacuaauucuagcaaugguuaggaaaacaucuugguccaguaacauuuuucacucagcuaauaauuccacaguugcaaggcucauuccugugucuacaugugacaaauaacauguagaacugcagccauaauugaaaacaaaaucuaccucuguuucauucgcacaauuucuagggaaaaggagacuuuuuuuuccaguaacaaaacugucauauagauuuuauccuaggagaaaaaauacaaacaauuuuauuucuguaagaauuauuuggcuauggugggccuucuggucuaaggggucccauuguugucaucuuuaaagauaauaagaauggguaggagaucuacucuuaacgaaaacuagagaauuacauguucuggugucagggagggagcaguuacuuuaugugggcauuuucccugaagcuguuauuuaggggauuguuuucucugaccuuugaagcacuuuuaauuuuuucaacauguaaagaaaggccagcauuucaucucacaacaauuaugaugaaaauagcucuaaguaaaagugaguugaaaggaacuguccucaccacaugguucuauuguauauuguuuucaagaaggcauuuuaaggagagagaagggaggagggaagaagaaggggaaaaaagggagacagaaauagaaguggggguggggagagagagagagacccagagaccaagaacggaguaagcaaaaugcugagugaagauguaaagggauuaggagagagggugcuaagagugagaggcuugucaucaauucuuagcuucauccgugggucugugaagugugauggagaggcagcauuaugcagugcaggaauuaagucuaaguuauagaaguaucuuugcuucuagauccuccugacauauauaaucucaauucaaaucuuucucucuuuaaagcccucuucgaacauucagguaagcuccaauuugcuuuuaagcuguuuucucccugugggaccaggaucuaaucagucucauuauggauagcuaugguacucauucaaccuccuugcaaccuuuccuuugggaacagcccuuccuucagcucguagcauuuagauggagcugucagucagggcguuacuccucucccacagaagcuggacaaaugaugcuuacuuacccaucagagaccuuuugggggcuggaauggauguuuggagccagagaugguuucucuccuuaugaaauuuugaguuauaaugaccacuuaaguuuaaaaugguaauccuucccaucauguaaagagaauccuucagcaaaugaaagcagcacugccagaaaucagagccaagucuuggagggagaaucaguuccugagaucauucacagaaugucugaauuuagccagccuaaaggaagacaauaccagaccuuuuuaucugcagcagccaauagacuccaucuuaaguugaggcuaguuugagcuugcauucuuuucccugcaauggaaaaggccuuagauauaauaauauuucuuuaauauuuucuuaagauggauuucaauagaaauuagaaucuacugaaccugcugacuuauucuacugcaacguaaaccaugugucuuggcuacauuucagcauuaacaaaucugcacuacuuuucugguuccucccaguaacuuuauucuacuuguucugugacacuuguguuaagcgauuucugccaaguuugguuacucuucucugagagaccuaacagauaaucuuauuuucauccauucuuagcaccucgcaguccucaauacccauccguaacucuagucaauagaccaucagugauagggaaccaccuacaacucuagaauaucaaugacagggcuucuggaaauggugucuuaguccauuuggauugcuauaaaggaauaccuaggcugaguaauucagaauggaaaaaaaaaguuugauuuacaauucuaaugaccaccaaguacaagauuaggcacugauagagaaccaccuacaacucuagaauaucagugacagggcuucuagaaauggugucuuaguccauuuggguugcuauaaaggaauaccuagacuggguaauucagaaugaaaaaaaaguuuguuugauuuacaauucuaaugaccaccaaguucaagauuaggcagugauagggaaccaccuacaacucuagaauaucaaugacagggcuucuagaaauggugucuuaguccauuuggguugcuauaaaggaauaccuagacacucauguuaagugauuucugccaaguuugguuacucuucucugagagaccugacagauaaucuuauuuucauccauucuuagcacaucacaguccucaauacccauccguaacucuagucaauagacuaucagugauagggaaccaccuacaacucuagaauugauuuacaauucuaauggcuaccaaguucaagauuaggcaucuagugagggccaacagcugcuuccacucaugguggaaggcgaggaggacccugcaugggcagagagcauacaaagagaggaagcaugagagagugaggagguaccagguucuuuuuaacaaccagcucucuuggugaacuaacagggugagaacucacucaucaccaaaagagagcauuauucuauucaugagggcucugucuccaugacccaaauaccucccacuaggccccaccucccaggacugacacacugaugaaauuucaacaugagauuugguggugacaaaugaaccauauccaaaccauaagagacagcaagaggaacacagaaauggugccagacaguaaaaggaauuuugucaccaaugugaggucacaucgaccauucuccauggcucccauguagauauuuuaugacccuagcccacguggaucacaucagacugguaccugucaaauuccugccuugauuuaugugaauauaacucuauauucucuggcaaaagcacaugaaaaaagacuuuuucuucuuaaauaaaaaugggcccuuaaaaagaaaaugaugaccagaaagcacauguuccaaguggaaaagguuaacacugaaaaucuuuaauuuuagggauuuccucaguucuucucuuaagcuucuauuucucgcuuuccauccuuggguuuuugcuacuuauugcuaccugcacuagagagcaaauaaaaugaaaucuaauuuagaguggacgucugcuaccucacagugcccuuugggaaauuuuaguacaagaggucacuguuuaaacacuuugcuauacaucauguguggggauugcagucaaccgagauaccccugugcaguguacacacacccauacauaagaacaugcucucaauacacacagaugugcacauguaguaaaauguuugaguuccccaggggcuuucuuagaugggaaugagcagcccuacuggccucagguuuuggauguggcauaggauagggaguaugggacagaaucccuuuccuccacgucaguaaggacgaggcuccaccccagcuggacucucagggaguugagguuccuuucugcugcacaucagcacucccugcuucugccccaaaucagcuuugcccaccuucccacugccacaucauccagcugcuucaguggcauguuccuguucugcauggcaggggacccaacucagcccaugaagccacugaaauacaggaaagagcaucuucauugaagccacuggagcaugggaaagagcaccaccagcucccugcucgcuagcugugaaccucagguacucuacagaucuuugaguuguuuccuuaauuuguuaauugagaauaaaaaugacugccuaauaagagucuugugaggauucaguacaauaauauacauaaaaugcccgguuuuuguagauguucaauaaaggucuguauuagucuguuuuuaugcuguugauaaggacauagccgagacagggcaauuuacaaaugaaagggguuuaauggauucacaguuccauguggcuguggaggccucacaaucaugguggaaggugaaaggcacguuucacaugguggcagacaagagaagaaugagagccaagugaaaggcauuuccccuuauaaaaccaucagaucuugugagacuuauucacuaccacgagaacaguaugggggaaaccgcccccaugauucaguuaucuuccacaggaucccucccacaacaugagagaauuaugggagcuacaauucaagaugagauuuggguggggacacagucaaaccacauuaaggucauuucuccauucuuuucuuuccauucucaaagacagucaguguuaaaacucuuuccauaaguuuaauuuuccaugucaccuuauaccacuaauuaugccuggccuauaguagauacuccauaaauaaauguuuaauaaacaauuaaaugaagucuggcugaucauauauuaaaauauguauuuguagaagauuacugcaaccuagguccuuagacaaauggaguuggaauauggcggauauaacagggugagcaugugccacguuuuucagaaggagcccaacaaugugccacauugcaccaggugaccugaagagggcugcugauuuucuguggucaacaccccacgacuucaggacaccagcagggcugguuauguuaauaggacuaugcuaccagcucucucucuucuguucauaucccugcugcuauauucuagagaagccuguguuucuaucuuugcuuggccuuaaaaccagagguugggaaguauggguaauaaggucugcuccaugcaguccucuaugcucaguggcucugagaaucaccaaguacaguguaguuucccacugagucaguauagauguccuuuguuggguguccgugcaacugucacuaccuuaugccccaucacucugugccauagcaaagaugccuuuugccuucaaccacauagguggcugagcaagagagggcuuuggcuugacagaagaucucaagcauuugcuuucugccuucuaguuugagugguuguuguugcucuuuucaaagacuucagauacagggacaauguaaaugucagucauucugguaauauccuugcauauguggucugugaggcagccauccuugggagcuugugccugugucugaauuauaagaaagugugaugacaauggcacuccuguuucaaguuuuagguaucaguacugccauggucacaccugggccuaagaaaccaccuauuaucuugguaaggccaauuaagccacaaaacaugcaguguuaaagaauacacacuggguacagauguaccacuguggaaugccauguagcaaauauuaucuugaagcacauucuaggguagcaaaaaguguggugcagacagccuagaaaugagaaucagcagauaagaacugcgguuccaccauaacugcuaaccagcuguuugaccuuggcaaagauacuuaaccucuuuggaccuuagcuucucuaaguauauaauuagacaucuggucuagacaauuaguuucuaaaucuccugucaacucuaauacucuaugauuauuucggcccauagucuaguaauuuuccaagacaaguccaauaauaagaaccauaucaccccuaaguaucuaauugguuaaagaagccuuggauuuguugacaacuacgaaaaugaggaacuaaaaagccagguuaagggaaauuaauacacugaugaggaugaaugugagaagacaacaaagacuggaauaagugcuuuucaaggacuggcuccaagggaccugaauuugcauaagcuuauccgaaacuaaaauaagcccaacuuccuaaagaauuaaauauuaucugugcaacuaggacuuuucaauacgaugugcaaggcuuggaucagaauuccuuauugguagccuggaugcacuggacuucuuugacaagagauacaaagaauaaaauaaaucaagcaggacagccagagggcauuucucaauauuuauaaacuaaaaaugaagaguucuggaaauaauauugaaaauaaaauaucugauucaauuuucucauacuuaauaaauuuagguaacucuaggcugucagauaggagaauauguguccacauaaauagaaaaguacaaggaauaaaauuuuguuaccaauaaauuguuugaaaacuggaauuccacguaugccuauauauuuucccuagucuucucagcugaacuuugauuuccuagaaguaugugcuuuuuauuuaauaguucuagauauaaauauucaauggaucuaaaaucucuaugauguacaguccccuaauauuuacaacugucugcaacuggauguugaucugcauuuucugauuguguuucuaacuacccaugaaggaaugaguuuuauugauaaagaggauaaacuuaugagucaagccugaguuugauguuaacucuaccauucacaaauugugaaccuuggucaacguauucaaauuuuuauauccucagguucuuuaucuguaaaaugggucaggguuucucagccuuggcgcuauugauaucuugaguggugacaugguuugucuguguccucacccgaaucucaucuugaacuguuguucccguaaucccuacaugucgugggagggaccugaugggagguaauugaauaaugggggcaggucuuuccugugcuguucucauuauaguaaauaagacuaaugagaucugauuguuuuuauaaaagggaguuccucugcacacacucucuugucugccgccaugcgagacaugacuuugcuccuuuugccuucugccaugauugugagacuucccgagccacguggaacugugagucaauuaaaacccuuuccuuuaucaguuaccuugucuugcguaugucuuuauuaucaguguggaauggacuaauacaggugagauaauuuguccuuuacucaguagaugccaguuggauaccagcaguugugacaaccaacaaugucucuagacauuguucuguguucccuguggggcaaaaucacccuagguugaggaucucugaugcuuaucucaacugggguaaucaugcuuaucuuauugugucacuggaaggauggaauagaagauggauaaaaagcuucuacugcagccugggcccaguucacuuucucucuuggcuccaauccugguggacacaguaggggcuccucaaaagcuccacccaucaccuccuugaaucacugcaucaucuacuucacacuggggcacagacaauuggauggcuucuuugaucucugucuagcacuugaucguaaguauuggauuugaaaaccuaacuuuaaauaaugugaggaagaggauccaagguauguuuuauuucaacauauuaaaaauggauaucagauauaaaaauacugguuaaguuauauaaguagaauuguuuccagaccauauuguguauuaauuauauccuaguucauagccauaaaacaauauuuuauuauacuacauuuccgguggucacugagcauccaugaguauaguaaguggaaaaaugguaaaaucugcaaaaugguucuauauuagauuuuauuaaauaagagaagcaacaacuuaaaucagacucaaaaaaucaaaauaauuuggcuccuuuaaaaagcaaagaaagaucaacaaaaaacuaacauuuggccgagguagcuugcucccuuguggauucagagcugaaaguuacugaagacauaucaguacaaaugugcacauuagauaauucccacuaaagcaucucuugcaaauaaaauuuauggacaucugacagcauuuauugccuguccuuuauaaacacaguacuuaacaauaaaugcccucaaagaauucuccaguucucagaucccaccacagaaggacuuaagcuucaaaacuacuggcuuucuucugaaaaccuuuuuuucauccaaugaauaccaaaagaaccucuuuccugaauaaaauauuagcugaaugguuuacuguagaauaaucccuuaucuuagcuaucaaugcugaugugucauuuaugguuauuuguuauaugauagauaaaauguuugaaugauaaauccuaaauaaguuuugaauauguauuauauauaauaugcauauguuauauauauuauguaaaauauauauauacuuuuuccucccugcuccaguuuuuuccaucucuaauaguuguaaggauaaaauuugcaauaauucuuuuacaguguuacagaauucuaaugugaauaagaaaaaaaggaauaagacuggaauaccaagccaugaacuuggaaagaaauccaaacuggcauacuggugaauacuacauuuucuugaaccaaugugccaacuuggaacaauucuuuaacuuaccuguuucuauuuccucaacuuauaucuaucgguccucaaaauagagacaugacaaacauacacauucaauaauuauacacauauacacauauaccagaugcuguagcauuuacaaugaagucacccuuugcaaaucauuuucacauaugucugcuaguaguuaaaaagauaccuaacuuuauuggaggaaaaaauaaaucaucuauuucugcauaauuaagcuaagcucaaauauguuuucugaucccaaucuaauuguauugauuucucacuuugccuaagaccacaagaaaacauucacuacuuuugccaccuuuaaaaucuuugauccugaucuucuauguuugauggugccuacggcucucuuaugcucaugacauuuuagugaacuucacuaggugacuuuguuggaauaccucuuggaaacaacuuacuuuauagaaaauaaguaaaacauagaaauauagcaugcaagugaagagcauggacucugggaccauauugccuaauucuaaugguggcucagccucucaggagaugugcugaugggaaacgucauuuaggcucucucuguguuccucagcuuccuuaucugcaaucuggggauaguaauuguacccaacucacagaguugauaugagaauuaaaugaguuuaaaauuagaacagugccuggaacucugaaguguuggcuugcuauuauuauacuuuacauaguuaauauuauaauucagcugggcuugaccccaguacuuucuacagguguaaggaugagcaaaccccuaccccucucaggcucaguguuuugaucaguaaaauugaggugggaugaaugaucucuaucccugucauuugguuaggguuuacugacaaugccugcggcagugccaugggaguauaaauugguaagauguuauggaaggcaauguugcaguaugguuuauaauguaauggguucauacuuuuagucuugggaauuuguaauguagguaauuuaucuuacaaaaauacuugcauacauacauaaagauaaauauauaugaauguuuauuaaaacauguuuaaaaaugaccgcaauaugcauuuauuaguaacugguuaacuaugacagauaaugcuacuuuaauuuauagcuauucaaaaggaguuuggaaggagucauggaaguaaacagccaaguggaaaaacacaaguauguauaucauggucacauuuugaaaagaaagaaaaaaaaggaaggaaggaaggaaggaaaagugaaugaaagcaguaaaaaaauacauguguaucuuagaauauggacagaaaacagauuuuaaaaacaugugccagacaauuaacaguaguuaaucuggaguggauuuauugaugucucuaucuuuuaugucauguauuucuuuauucucuaguuuuuuuuuuaauuuuacuuucaguuuggggguacaugugcagguuuguuauauagguaaacuuggguuguggguguuuguugugcagauuauuucaucacccaggaauuaagccuagcacccauuauuuauuuuuccugauccucucccaccucccaccuuccacccucugauaggccccagaaugugcuuuuccccugguuuagcucuguguccccacccaaaucucaucuugaauuguagcucccauaauuccuacguguugugggagggaccaggugggaaauaauuugaaucauggggucgguuuccccaugauacuguucuugugauagugaauaagucucacaagaucugaugguuuuaucagggguuuccgcuuuugcaucuucuucauuuucucuugccaccaccauguaagaagugccuuucaccucccugucaugauucugagaucuccccagccacauggaaguauauguccaaugaaaccucuuuuucuucccagucucagguaugucuuuaucagcagugugaaaauggacuaauacauccccucuauuuguccauaaguaaauauuacuuugguaguuugaaacaaaaagcuguuaaguuuuucuuuuuaagaacaaucauagacuuuggaaucagacaagcuuggguucaaauuguaucuuuauagcuuuaaagcagugugaccaaguucaaacuauuuaaacucucuguucuuauuuccuuaguuguaaaauggauaauauggaacucuccuuguuacauaaaaauuaguuuagaaaguacauuuaaauucacuaucacaaagccuggcaaacuguaaauguuccauaaauguugguucucuucccuccacucccugcuaauuacaaaauaucugcuuaugaagcuguuuauaguuuaaaaauagaaauauauuucucuacaaagauuuuuauuuagcucuaggugugacacaaauuuaacagauugaauuugugauaacuuuuaccuuaaauguaucauuuuuccuacgaucagaguuauuuuaggaggaacaagaaaauauaugaccuaagaucugaaaaaaaaugguuaacauuuagcacuauuuuacaguagguccauccagaauaaaaguauggguguccacugugucucaauccaucuauaaaacaguaaagcaccacauagugaagugagaguuauuucuauauauauauaugcagaaaaacgucugaaaggguaaaagcaccucagcaucaagugucuauuuuucagauguaaaauuacaaguaaauuucuucugagcuuuuuuauauuuuacacauuaacaaaauuuaugucuuuugaaauaaaaaugcacaaaauuuuguuuauaccuaaaauauguagaguuauacaaugaugaaaaauaaaccauggcccuugauaaugauuuuacuuuaauuuucuaugaaaauuagcagggcaaacuuucaagcuaaguguuuucuuaauuuggaugaggucaacuaaauucugcaaaggaaguagagacaauggauuuugacuuguuucuaauaugaggaaauacccuggucuuaucauuacaacaguggauuguuacuuuuguuuuauuguaaacaauucaaaaacacauuaaugaagaaauugauggguauuauuuagggaauagcaucuguuuuuuuugguuugacugagucuguuaugaucgaauaguuuaguguauuugugagaagggaauaaccaucaaaugcucacucaaaacagaacuugacuaaaaaguuuggguaacuuaaugcaaugggaaauuguuaaaaaugggugaaggauguuugucacucgcuucagccaaaaugaccuguuucaggcugaaagcaugagccugcaacaggugggagagugcgugagaaaguaugaaguauuauacucuuaauauguucaaauucaaaagcauuuucaaauuaaaaaaggauuuaaaaaucauacaguuuaauuucuccuauuucuagaaaagcauuugaguugaacuugaacugguucuggaacccagaucucccaucucaaccuucuuccuucuuuccuuuccacuaaagcaugcuaauguaauuaacauucagguaaagucacauggcacauaggcugaagauuacccagacagacuucagauccuacugaaagauagaauaauuaauaugauggcuaacaggauguuugucaaaauuauauuuuuggaauuugauuuuuuuucaccuuuaggcugaccuauuaauuauagucaauuaccguauguacacacuuaugaggaaaaaacacuucuuaguguugcauguccaaaugaacccacugugcugaggcccuauaugugucugauaaauuacuaacagaugguuguuuauuauauggauuucacagcuccucugccucucucucuuauuucugcugaguauucugguaacauaaccauuucucucuaaagacagaaagaugaaaucagaguaaggaaacucccaagugguccacuuuauucuacugaauauuuuaguccaauggcuuggcagagaggaccuuuguagauguugaugaaaacaaucuguuacaucgcugucacuguucauuaagagacuugcaaagaucaaauauccauaugucauucaagaagauagccaauuauuauaaauguggaauuuagagcuguguacugaacaaauucaaacuccacaaagcuacugcacugauuuuucuaaaaccaucaccauaaggaaaauucauauuccuggucacaaacacaaaacucuuuagaucuuacccuugcccauagccuugacuucaugcuuugccccuucuugccuuguauuaauacuuaaaaauccagcccugugaauggcguuucuucucaagucagagcaugcauccaccugccugccuggggcaugaaggacuuggacuugaaggacagcaugauaugcuccucgacaaggucuccucccaguuacuccgcauugaacaucccuaucacaaggaugcuuguccaggauugagucucauuacuaaggucccaggaauagauguuccccgcucaccucucgccaccccugccacuagccccaaguaccaucucucaacucgaggucucuacccagccaugccuucuccgugguaggcuccucagaucucuucauucaacuaguaaccccuuucucaucccauaaauuccccuucaagaugucaccuccuugaaaauacagaagcacagccagagacugcucauggccccucuaucucaaaccccacuucacugaaucagcccuggggugugccggcuggcucuucuccaugcucuauggcacucagugcuugcauguucaacacauuugcuguguuccacuguagcacagugauucugaguguggacucuggaacaagugccccugggcucaaauucuggcucuucauuugccagcaggauuacauaagacaagugacuugcuucaccuuccuuauuuguaaacuagacauaaaaagcaugaucugccucauagagaagaggauuaaauaaaucaauauaucuaaagcuuacaauagucagcccuccaugagugacagcccuccauaagugacuguauuugugcuucacccuugaugucaggauuuaguuuucccaggaaagucuuacuuauuuguauaaccauugccccucacagagugcuuugccucuaguaagcuccauauguacaguggccaaacugaacuaguaagaauaaggggaauaaacucaaggugagaaaauccuagaacuggaauuaucuaaacacccagguuuaguuccaaauucaugaagcaaagaggagaucuauguaggcauauauggccauguuuuuuuuuuuuccaggggaaacuauuucagaugauucuacccuugacuguaaauguggucuucuccaacagcagucaguauguagcuucugacuaauaccaaaagguuucaugucuaugcacgguauucuauuauaguuuucaacagaaaguacaaauuaccggaugccugauaaauucauccagaaacacuaagaaaaacagaugcuauuuuccaaaccuuaucaaaugauucccuuucuccucacucaccaggguaguuuggggccaaauaugauggaaaaguaauuugcugacuucugaccacuugcacacuccagagggaggggcuggggcaguaaggcagauauaucaauuucccacacucuacucucuuccuucucugcgugguuaaaaacaaaacaaaguauuugauuuccucauuuuuucuacuaauggaagaugacagauccaaugguugagcucacaaaugaaaaucuuuauuuuguaugaucagaaucuuucuguuuccauuguaaccaauccaauaccagagagauauuccaaaguuuggaaggauagugcaagucagcuaugaucuagcauguacugaaggacuuuucaccaagaugucagcucccuacacacucagagacuuagcuagcuuugaccuugaagccauugacagggcaugcaaaggagagauaauggguuguuccugcuuggagguuuguguucuagauauaccuuuauuauauuuuagacauucuucacuacuaaaaaguaaagugguuaccuuagggggaggccacaggguagcuacauauaggaaauauauauauuacaaaaaaacuagccaaggaacuuuguguguguguguguguguguguguguguguguguguauuaguuugcagaaucuuuuuuuccauuuguuauuuauuugcgugagaauagcaauacacaggcuuauauuuauccucagcccucccuagcccucccaaacuauauuaacuaccugacucuuaaaaagaauugugaaggaacuaaaaugauaaugcaaagagacauggaaaauaauauuuauuuaauuuauauggauuuagccuugagaauuacuuuaugcuaagagacucagggugacuacaacccccacucacgggcagaacaagaggcuaaacauguccuuaugcugcagaguauauuucaaacuauuagagugacccauuauuucauuaauuuaaguacauuucugguugcugauaaaaugaguucuagccuguuggcggacaagaagagaagaacaaggcuuuauuccuaugguuaggugccguugcgaccugaggcacaugcuauuguaugagugauucugacaaagagaauaucuguuuucaacuugcacuucauuuugcacacaggcuaaaauaggaaaagaggccauuuaugcaucacuuucaaacacaaaguacuugauagagaaaaaaaaauucucugaacaguggucagacucuccaaauagacaagcugaugccaaauaaaaggagugauugugaacggaccuggcagcacguucuucaggaaauggccggguguguguauccuugcugauuacauguauaauagcuccacaagcaaugcugucuaucaagaaaggcugcaucucagagucguguuuucaaaaggaaagagaaugagcacucagcugagccugucuggaaagaggcaaagcggcucuaggagaggggaaggaguugggaguucuuacaugcuggcgacagccaagccgcaagagaugagcagggcacagagcacacguucaaaggaaaauccaguguugacuuuuaccucuacaggauguuugaaaaaggauucaggaaaaucaaaacucaaaagcauagaucagccuguggaaaguccagcaugucuuuccacuuuauagugaacucucuggaacaaaaagugacauucagauuucaagucacagcuaggauaucugagccucauggguaaugggaacauguccggacaacauugggaaaugagugggcgcugcccugcaagaaccgaaugggauacaaugcuggaacacacuugccacaaugccuagcaguaaguucuaugaaaacauuaucauucccaacauuaauacuaaucauuauaacaaacaucuaaauuuuaaaaggcccuucuaccuuugucacccuguaaaugacauggauaaagucauaguuuauguggcugugacaccacaaagacccagggcugauuuaccuggcaauacccccuuucucuacggaagcugugguucaaagggaccauagcuagaagcuacucugcaagggaggcuaugagcuuagcucuuagagcccaacugggaaagaauauuguacaauaacgucuacaaaauuacuacuuuucagccuggaaucacguuugugaucuucuuuggaacuuaucaaugcaaagcuucauaauaacggaauguuuuccuauuguugcuuggauugccuuaaucucuuaccauaacuuuuucaucccccaacauuuaaaguuuuuguauacuaaaaaguaauagcuauuuauuuaauuuaaaauauuuuaccagcuuugaaguuacaguguguggauaucuaagauaucaaucuaagcucuaaucuauacaggcagaagugcuggugcuugugaaagcaggaccuggaaauagacuggguucaaguccuaagucaacccccacuagcucaggucagauaaguugauuaaucacuccaugcuucaguuuucucaucuguauaaugggaaaaaugagagugcuucacucaugggagaauugaaggaguucuuaauaauuaucaagcucuaaaaacagacgaggcagaagcuaaaagauuuuaaugaacuucacauuuuuacagguaugaauuuggguaaacugucauaauaguaugaaucauuucaagaaguuaaaaacaaacaugaaaauuugcauuuugaauuuguagggaugucuuaaucuguuuccacucugcuaauaaagacacaccugagacuggguaauuuauaaagaaaaagagguuuaguggacucacaguuccacauggcuggggaggccucacacucauggcagaaggugaaagacacgucuuacaugguggcagacaagagugaacuuguccaaggaaacuuccccuuauaaaaccaucagaucuugugagacuuauucacuaccaugagaacgguaugggggaaaccacucccacaauucaauuaucuccuaucaagucccuccuacaacaggugguaguuaugggagcuaaaauucaagaugagauuuggcugggggcacagcaaaccauaucaaggaauuuguuucuaauucgcuaauucuauucauccauucaccaaaugacuauuagccucuccuagaggaucucuuucagaugcaaugaagagaaaaaacaaacugauacuaaauuguaauaggcagaucaagagaggagucacacacugcauucacugcacaguuggcuuuaauuaaauuuggcuuuaaguuugcaaauauuaaaaucacaguccaaaggauaaaaggaguaggugaagccuauuaagguuuuuaaugcauggaggaaucaccugcacagaccauguaucaguggaguucaugaaacugcacauuacuagagcaaucauuuauacauuaagcgcuaagaacacccaaagaaacauuccaucucagcagcugccaucuuggauuuacuuuuccaaaagagacaauaauccagacuauagcauuugaaaacauucacucgggcuuuguuuccuuuuaaacagucaaaaauucagugcuccugaaaaauauuguuugaaguaauugaggccccuaaaguuucugcagaaaaaacaggggcucuuucuuccuuuuucaugaaauaaauuuugggcucucagccacaacaaaaucccacaguuacuuuaguuucauuagaaagguuuacuuuugacuuaaaguucucucuuaguuaaaugauuccauuaaacaugcggccggggaugguggcucacgcuuguaaucccagcucuuugggagacugaggcaggcagaucacgaggucaggauuucaagacuagccuggcuaacguggugaaaccccaucucuacuaaaaauacaaauauuagcugggcgugguggcaggugcugguaaucccagcuacucgggaggcugaggcaggagaaucgcuugaaccugggagguggagguuucagugagccgaaaucacgccacugcacuccagccuaggugacagagcuagacuccgucucaaaaaaacaaaaacaaaaugcaugcaugcauauacacucggaaaacuguuuaagcuucaaagaauguugugucugccauauuguguuuuuuuuuuaagaaagguagaauuaaguaauugagguaguccugaugacacaaagcaucagaaaagccuuccaaauuaucacagaaagaacagauuauuauaaauaauuauucaaagaacauaauuguacauacaacaauguacacuuugauauuuuuguccaauacauuucuagcaguacaacugcuggauggaguaucugccuguugcuguguaaauaauuaucauaaacucaacggcuuuaaacaaagcuauuuauuuucucacaguuuuugugggucaggaguauuguuugggcacucagcaagggccucacaagacugcaaucaguguuagcugggcuguguuccucuccagugcuaagcguccccuuucaaacuuagguguuguuagaagaauucagguccuuuuggcaguaagacuauagccccuguuuucuugcugucugcccacugcggguggcucucugcuccaagaggcucccauaaccucucccacaacauggcaguuuacuucuuaaaggucagcaggaggauuuuucucuuuaacaaggcccuagucucucuucugagggcuuucaccugauuaggucagguccuccuuggauaaucucccuuucaauuaaaucaaaaucaacugauuugagagcuuucugauaucugcaagguccccccaccuuuuccauauaaugaaaccuaaucaagagagugaaauucuuucauaugcauaggucuugcuugcccuaaaaggagugaauuauauacaggauguacacagaggacagauauugugggggcugauaugguuuggcuuguccccacccaaaucucaucuugaauuguaguucccauaauccccacgugucaugggagggacccagugaaagguaaucaaaucaugggggugguugcccucaugcugcuguucuugugauaguggaugaguucucaugagaucugaugguuuuauaaggggcuuucccugcuuuugcucagcauuucuugcugcugccaugugaauaaggauguguuugcuuccccuuccaccaugacuguaaguuuccugaggccuccucagccauguugaacugugagucaauuaaaccucuuuccuuuauaaauuacccagucuuggguaugucuguauuaguagcaugagaauggauuaauucagggaccaucugggaauucugccuaccacacuggguaaagcagugcuuccuuuaaaacucuggaucaguaugcuaaauuguccucuaaagagggugggauaauuaugaguucaccaagaguguacgauuugcuauuuucucauaccucaucaauauucaagauuauugguucuuuaauuuuccacccaaucugguagaacauuuuuuucuuauaauuuuuaauuucuauaucuuugaaaaaaagauugagcauaauuucauguguucacaaaucugugaauuaacuuuuuacaucauuugcuuucauuuuaaauggucauuuauuuuguuuuuacugauaaaagcugucuguauauaacagaugacaaccuucucuuuuuucaucuguuacaacuauucucucuucucuuucucucucucccaagaauguaacuugagcugugucucauuaaugccacuuuuaaagaaaaaucagauuuuaagcucaaccagucagaagcagccaacaaccuguauaacuagggacuuugcaacaagauggaccaaauaagacaacgguauaauuguaaccaaucaaauauuuucuuggcuuuccuuugcagguuaucauauaaaagucccccuacccuugcauucucuuggugaaacuccagaacaucuucuggcuuggagcuaccugauucauaaauugcuguuugcucaaagaaacucuugaaaauuuuauugugccucgguucacauauuuaagagauuuugugacuuauauuucuuucacucuuuuugccauggagagauuuuuauaacaaauauauaauagugaaauaaaaacaauguaaacuccauuaauuuguuaaaaaacugacauguauuaauacucuucaaacauuagaagaugagaugaauuuauaagcuauggauauggaaagaagaacucuauuacaaucaguagaaaaacaaguuauaaaaaaggauucauucauugugauacccugcuugaucaaauguuuguuaucuacauauuugaauaauuguguauucauuuaaaaauguccagaauggcacaugccaagauauauccacacaccauaguggauauauuuggagaacuggacuaggcagauggcuuugacuuuuuacuuuauucauuuauguauuuuuauuuuuauuuuuugagacggagucucgcucuguugcccaggcuggagugcaguggugcaaucucggcucauugcaagcuccgccuccugggguucacgccauucuccugccucagccucccgaguagcugggacuccaggcacccgccaccacgcccggcuaauuuuuuguauuuuuacuagagacgggguuucaccguguuagccaggauggucucgaucuccugaccucaugauccacccaccuuggccucccaaagugcugggauuacaggcaugagccaccacacccagccucuguauuuuuaaauucaauuuuguaauaaauaaucuuugauuuuaucauuaaaaagaaguuaaguaaugauauaauguuuaaucuacuuuuaauaugaaacauggcauuuugcuguaucuuaauuauuugaauauuaucauuuuaauauuuaacagauauuuauuuagcaauaugagcuccuuccaauuauuuauacuagauggguuaguccuaauuuucucagcuuguaagaggagguaaaaguauguuacuuggcuaggggucagggguugguuaguauguaaaggggaacuauaaccuugacacuuaaaauccuaucagaacaucuauuuuacaugucacccaucaaugggaacuuauugaaugcauucaaguuguauuuggcugguaugugcagaaagacaaacuagaaacagucaauucaccgcaucagaagaguacacagaaauaaccauuaccagcauaaggaaauuguccaaaugugugcuuccucaaguugaaagaaaccauuucucagaaggaagaauuuucacccagcaguuaaaucagagggaucaacacaauuggguacuaacuugugcaaggcagaagagaaagugggaagcagucccaggcugcaggauggcuucaugucuggaagcacagcacagaaggccgauggcuaagcuggaggcugagacguguccuaugcaaagaaggacagauuuggaggccacaguggcuuaaggaaaaguuguuucuuauagggugcuuggccauugagcuuucuuauauucaugacucuuagcuauuucugaagucuguuuuuagggagaggaugcaggaguggucugugaaugauugagauuuaucccuuguacagaaauauugucauuuuuuuuuucuguggaagcauggguucccugucaagcacaccugccuuggcuuccucccugaggcucuaucauguuuccuaagguagacacucccuucauaguguuggguuuucucucacacucccaccuguauccuccucuuggguuugagugaugcucuccagagggguaagacacugggggcuguggccccauaauccuuccugacuucugaccauucaucuucauguagcugcauuauccugcauacucuauacucagcuaugcucuaccgaucaugagagauggccuggccaccccaccugguuuucuuucccugcuccacaggaagaauuggaaagaagccuggggaacuguccagcagugccaagagcugccgugcaggauuaggaacaggacaggcuucagaauauuacugcuguguauuuggagccacucaagcccaacuccucucacaguggguuuucacggggggcaucucuggaaagaaggagaggcuugcuucagcagcuaaguggaagcagaccuccgauguaagcaguggagguaauacuuccccuucccuggcacuugaauggaaaagauugaagcaaaaugaaaggccaaucuguaugcaauuaaaugcuucuacuggcaauugcuuuuccuuccccugcuaaaaaagcagacauccaaauuucccaccuucuuaguguguccccccuccugugcuaaauugccacaguugggaguagcgucuucaggcaguuacuggcaagaauagaugcaucaaaggaaugaagauuuuuguuuuugcuuuuaacuuaaaaaaaaggcacuucucuagagauguaguuacuaagcuuuuuagcuuaaucuuuagaaacuauacauauauauuuagaaacuauauauucuauacuauauauaguuucuaaauauauagcuuuauauguaaauauaaagcuuaaauauauauaguauagcuuaauacacauauuaagcuauauauuuaggaacuauauugcugcuccuguguguagauucuucauuaaagauuauucaucugacaccuucuuggguuuuaaauuagcuuuaagauuuuaaaauuucaaugaaaccugacaauaaaaucacaauuauucaguaauuugcaauaugucucaaauucuuagaaauuuuagagauuuuaaaguccucuuuuaaacugcauucaucagauauugcaauuugagaagagcaauuuauggacauuuauuauucgucuucugaacacuacuguucuuugcauucuuagacuauggggcuucaugcauaguuuuaauuccaugaucacucaaccaaacagaaggaaagauucauauuuucaauuuuauucauucgaauauuuacuaaauucauacuguguggcaggaauacagcagugaaaaacagaggugaaaucuaugaccuugugaagcuuucauuuagacaggaaagccagaaagacuacacgccaauaagcaaaugugugugugugcuguggugaaaggguuaugaagaaccagaggggguaauaaggagagaaaaaugggaguuuauuuuauacuguguauguuagaagaagagaagaauacacuuugacaaaggcaaaagcuuauaugcucaucauuuuauauuagcauauacaaauaucuuaacacuauacauauguagaaaauuugaggaaauaaacaagagagauagaaauuuccugaaaauuauauagagaaucaccuaaaaacauaagccugauauaaagauguuucaaaauauaaccuguuauuuguauacaaccucguauuuguacauuuguauauaaccuuauauuuguagcaaagguaaaacuuugccaaugauagguaguaauaaaaugugguacagcuuuguauuauucuguucuccuccugcuaaaaaagacauaaucaagacuggguaauuuaaauuuauaaagaaaggagauuuaauugacucacaguuccacguagcugggaggccucacaaucaugguggaaggcaaaugagcaaaauaacgucuuacauaguggcaaacaagagcuugugcaggggaacuucuauuuauaaaaccaucagauauugugagauuuaaucacuacaaugagaacaauauggaggaaacugccuccaugauucuauuaucuccaccuggccccacccuuaacaugagggcauuauuacaguucaaggugagauuuggguggggacacauccaaaccauaucauuacacccugggacccucccaaaucucauguccucacauuucaaaaccaaucaugccuucccaacagucacccaaagucuuaacucauuucagcauuaacucaaaagucucaucugagacaaggcaagucccuuccaccuaugagccuguaaauucgaaagaaaguuaguuacuucccacguacaauguggguacagguauugggugaauacgcccuuuccaaauggaagaaauuggccaaaaugaagcggcuauaggucccaugcaagucugauguccaauagggcagucauuaaaucuucaaguuccaaaaugaucuccuuugacucuauaucccacauccaggucaugcugaugcaagaggugggcugccacaaccuugggcagauuggucccugugguuuugcaggguucagccccccucccagcugcuuucacugauggcguugagugucuguggcuuuuccaggcacacggugcaagcuguugguggaucuaccacucuggguucuggaggaugguuucccucuuaucacagcuccacuaagcagugccccaguggggaugugguguugggucuccaaucccacauuucccuucugcacugcccuagcagagguucucuaugagggcuccgccccugaaacagaccucugccuggacauccagguguuuccauacauccucugaaaucuagacaaagguucccaaaccucaauucuugauaugugugcaccugcaggaccaacaccauguggaagcugcaaaggcuuggggcuugccuccucugaagccacagccccagccuuacauuggccccuuucagcuacagcuggaguggcugggacacugggcaccaagucccuaggcugcacacagcaaggggucccuggaccuggcccaggaaaccauuuuucccucauagggaaaauguauuuuacucuguaaaauacaaaguaguucaagcaagggauacaaacuuuaauauaucaugauguugggacauauuucaggaaugcaagaaggugguucaacaucagaaaucgaccacugcaagcuaccagguuaccagguuaaaaugagaaaaaaaaaaugaacaucuuucuggauucugcugaaagauucaacaaaauucauuuuuuucuauuuauguuuuuuauuucauuauucuauuauaaaacacucauuuauaauagaaacucuuagcaaacuauaaauggagggacauuuuaaaacugagauuuauucccaacugcuaacauaggucaaauuuaauagggaaacuuuggcauuucuaugaaccagaaacaaaaaaugauucucauaucauucugacuauucaacauuauacuagaaauuccaaccacuaccaaaaaaaccaaccagcaaaacaaacaaaaacaagaaaaaaagaaguuaauaguauaagaauugggaaaaaaagagcaaaguuauacuaacugauaaaugucauuacucuacacaaaaaaauuucaaggguaacuauagauaaacuuuuggaacuaaugggaaaguucuauaguguugucagauacaagacacacaaaagccagugacauugcuauaggccuuaauugacuaguaagugugauaaaaaguauaaacacaugauaauaaaauuuacuaaaugccuaugaguaucuauaucaaaaauauuacaaaauguuacuuaacaaucuauaacuuuuuuuuuuuuuuuuuuuuuugagacagagucuugcucuguugccaggcuggagugcaguggcgugaucucagcuuacugcaacguccaccuccuggguucaagcgauucuccugccucagccucccaaguagcugagacuacaagcgcgugccaccgugcccagcuaauuuuuguauuuuuaguagagacgggguuucaccacgcuggccaggauggucuccaucccuugaccucgugauccaccuggcucggccucccaaagugcugggauuacaggccacugugcccggucaauaaucuauuauuaaauagcaauauuuaucauauucgaggcagaaaauguucaauaucauaaugguguuaaucuucuccaaauuauuccauaaauucaacugaccacaaaucaaaaucccaaugaaauuaucuguggaacuugacaaacugauuccaaaauucauaugaaagaguaaagcuaagcucuauucuuaacuuggaaaagaaugaggaggagggguuuuuacuaacaggugucauaacuuacuacugaguuugaguaaauaagacugugugauauuggcagagggauagaaaaauagaucagugagacagaauaaaaagcacauaaaaaagacuuuucaaggcacacuugaagguuuuuccuuugcagccugaggucuucuaggauauuuacauugugacacuuucuccgaggaaagacugggauggaacaaaugucaguauugcuccuacagcauguacuugaaagcuuaggucauuucagccuaagaaauaauuuauaaaaaugcaacaagagaaggacauaaagacagaagauauccuacagaaauuuuauauuuuacucuggacaaaaaugguuuauucauagcccaaguaaaaaaauaaaaguuguaacuaaauguaaucaauguaaccauuuaguuguaaaagaguaagcagauuuuucuucuuuuauuaagcagucacccuguugggguaaaacacugcaacucuccuuccaaaaguggggggcuuggaucuacuugagcauugggagaaaaagaaggacagggaggagagguaggaguaagaggaagaggaagaggagaaaaagaaaaggaggaggaagagaaauaagagaaaugaucuuauuaaauaaauauuaaaucuaguguagccuaaauguaacgccuuuauuacuauuuuguuaguaaauaaauaaaagagaacaaauaucaacaacaaaaaauauccauuugguuguucuuagugcauuaaauauucauuucaccauucauuagaaugaaccaauacauugaucagggcacauacagaaguuuggaauaaaauaaaagaagcacuagaauucaauggaaaaaaagauauaugcaauauuuaguaaugacaaucugguuccccauauaaaaaaaaucagauagauugcuauauuuuaucguacaaugaauaaacucuaaauagacuaaagaccuaaauacaaagaacaaaauuuuauaauuuuagaauaaaauacuugagagugucuccuugaugucaggggagagaauuauuuuuaaaaggaacacaaaaagcagaaaucacaaaaugauagauaaauuugacuacaugaaaauuaaaaucuuuguaugacaaagaacacgaaugaaagauaaguuaugaaaaggagaaagaaauuagaaaauuguaugauagucaaaaaauuaguacucagaaucuaaguacuaacaguaggauuccuacaaauuggaaaaaaaaaaaagaugacccaacagaaaaauggguaaaguguaugaagaagcaguccauagcagaggaaacuugaguggaacugcaaugcaugaaaagguuuuaaaaacaaacaagcaaaaccaaauacauaauuauaguaauuuaguggcaguuacuguuauaaauacaucuauagauagagccauacuuagaauuuagggcaaaauauaauguaaagauaacaacuuuaaagucauuaauuuucaaguaguucaaacucucagugugcacuagaaagggaugugugagauaaacacagggcccagggagacuccccagggaagaagagcauuuugaauggguaguuaaggaggaagagaugucaauaaaggaagacagccuuagucgugggaguagagaaagaccaagaaaucaaggguaaaagaaggaggagguagggucaucagugccaaauacagcugaaauaugcauugauauaaagaaacaaauccuugcagaucuuuuacauucagcaacgaggaugcucaauauccgucuagggugugcuuaucuaaaucccuaauguggugcuauagacugaauguuguguuaccacaaaaauucuuauguugaaauccuaacccccaauguuauggcauuaggagguggggugccugggaggugauuaggucaugaggguggagcacucaugagugggauuagugcucuuauaaaagagaccccucgcaucuuccacaauaugaggacacaaugagaugauggccaucuaugaggaauugagcuguuacuaggcacugaaccugccaacgcuucaaucuaggguucuccagccucuagaacuguacaauguaaauuucuguuguuuauaagccagccaucuuguggcuuauauggcacuuuguuauagcagccugaauggacuaaaacagaggacugagcugccugggacaaccuggagccucauccauccacaucucccacuuggacaccaacauacugaacucugacuagaguaaggggacaguccugcaguaugacaccacaccagacaacaguccucuugcuccauggccuaacaacccaacuccacagaagcagcuucaaaaugucaggagaaaugauuauaucucuuuugcuccugaacaaaacuuauuuauccauuuauaguucuguauugugcugagugauuaaguuucaaaacucucucuaaaaauaaaugcuacugcugaucugagagaaacauaaaugaccacugagaacagaguuucaacgagcccagagaacugaagcugucagaugcagaccucacauuccuuucucuccaccaaguuauuuucaaggacaaugcacuauguucauggucuuugacuaacuugaccucuggcugccauccuuggcucucauaauaucuuaacacuaaacauacuaagaauuuuuuuaaaugggaagugauuagauaauaaauuaucuacagaggguggucuaaaugagcucuaucuagaauuuggcucaauuuuacuuugcuggauucaaucuaagcacgcuuuuccuaaaugccaauuguaaccagacauaaaugucuuguuaaaacaaguuuaucaucacuaaacuuaacuguagucucugauuagcucuuuagagccuaaauaaggcauauuuaugguugcuuuuuuuccucuugaugguaugucagcuacauaugggaaaaacuguauggcucagaucccuuaaaaugcuuuagaauuucuucuaugaacuuugaaagaacaagugucauguuuaauuuuauccuauauaugcaguuaaagaucaauuaaaaguauuuaccugcuuuaugaaaggauuaagauucucuuuaaguuucuaauauaaagcaaguauccaacuucaugagagacuaucauuuacauuucaauuaauguuggcuuuucacauuugaguguuauaauggcuuuagucugaauugggcaagugcucuagacuugucacuaaaguauuagggaucuugcugucauagauuaaugagugacacaauauuucagccauauacuauguaauucuaucuaauucuggauauuugucugcuggacacgagggaggggacuaaaaacacaaagucuggaugcuucacugcuuacuuauaagugaaguagcagaagaugccaacagcuaacaagaggggucuugccuggagaaagggugacgacacagcugauggcuaauuuaucccauuucucuugauaagguuugucacuaagguuccaucauaauauaugauguggauggccacagcauuuuguugacaggaggcugaaaccugacacaggccagucacauuacccucugcuccuaaugaagaguaccucugacuaaauggggcauacugcaucacacauucuggccuccuauugccucccgaacucaguucuuguuacuguucuuuuccaacuuguucuagugaauugcuccucacuguucuucaaacagacgaggcacacucccaccuagaagcuugacacuaccugucuagacuguucugaucacaaauaucuacacggcucacucccucaccucaugcaaaucuuuauuccaacauuaccuuccugaugagguuuaccuagggcaucuuauuucaaacuguaaacuguuucccugcuacccugaucuucccaaucucucuuaacaugaucuaguuuuucuuuuucuuuuuguucuguauagcucucaucaccuuucaaaauacuauauaauuugucucauuuuucauguuucuuauuguccgucuuccuucguuagagcccagaaaagcagaaaucuuucuuuuguugacugacauaucccaaagcuuucaaaacaauuucuggcacauaguaaguguacaauaaguauuugacagaugaauaagagugaaugaaggaaccugauagaauaugguacaaugauaagauacaagaaccaugaaaggcagagaccaucugaauuuuguuuacccugacauccucgacaucuaggacagugcccaggaauucuacuuuuugaaugaguaaaaaagcaaauaaauguuauugggaaggaggaggguuccuauaaccuaucuaaguauuaguggaaaaauaaauuauuuugaccaggcuggagguacuccugugucuaaaauccugcccagaguucuuuagcuuccugcagaucauaaaauuacccaggaugccacaagacauauaguuuuuuuaguuacuugagaacuucaaaggacuacaacuuaacagaaaguccuaacacaagugacagugccucuuaugcaaagcaaaagcacaugaagaugauuuuuguccuguaacagaaaugcuugggauuccauugcagucacuguccaguguuauuuucuugagugguaauaggagaugcccagcuuuagagucagaccugcaucagauggccuucauguucuauacugcgugacucucagaaagucacuuacauucuccaggccccuauuucucauauuuuaaaguaauauccacucuuccaggauacugugaagcugagaagcaugugucaaguaaaacgcuuaguagacauucaaaaaaagucacucugcucugcccucugugaccuuucuaaacaugauucuaaugguuccauucuaauauugguucuuaaaaauucuaucgcauauuauaauugcauaauaauguauaauuucauauuauaauucuaaauaagauuuuguuuuucucauucaaggucuaauuaucagaccacuuucuucucuuaauuucuucuugcauuuugcauuggugcuacauagaagaaaaaugccauaagaaauggaguuaaaguaccggccauuucauuucuuuccucaguucaaaaccugcuggugauagccaagagugguuauucaugcugaguuccuuaaaaauuauagcauucaucagaauauuuacuaaaagggcagagcaaugauucacaaaucacccacuauuuacauuggcaagaagcuaaugguguauaucccugauaagucaugacuugauuacuaagcaaaaguaaccauuaaaauaugaaacuguaguuuacaagaguagcccagcucauuacuuggaaggauuuccuacugauaugugauuaacacauagcuucuggaauuaaaggauaagcaauucagcaaccaaugaauuugcuaaauuuuuuguugccuacuuaggucuuuaacaauaccauuugcaggauguaaaucuuacugagaaaggagcacuucuguaaucugcauacucccagaagucuagguuuucagccucaaggcuacaaaacaggcaugcugaaagaaaaggaaaugagagcaugaaagcauuaagaacuuugucagguucacacagcuucuacguggcagaguugugauaugaacuucuugaagagaagccaguuaaggccacgggauuuauaaucagaaagaugugcauuuaagauugucuggagagcuucuccucuaucggguucagagaaacaauacuuuuaucccuuuuauguaugugucuuucauggaagauggagauccacugaaaaaaagaaagcuaccuaugccaaagaauaauuuucaaaguuuaaaaacaugacccuuauauaaauagagaaaacugguguaaaaauacucuuuaacaccuuaaugagggagccaaccagaugaauaugcugauucaaaagugcauaagaagaauauauaggcagagaagaaaaaaauuuggaauaagauuucugggugagauacaaaauuggacaaaaaucugcauauuaacauguaacaucacauaaucuaggcuuucaguuaauaguaauuggugauuaaaacaaaguuacguucccuagauaagcguacagcccuuagguggauuuguuaaccaguguuccaccuauaugauauugaaaugacaugcugcccucuguuugccuaauuuccaaguuuuggauaacuuuucuuaacuccugguaagcauuuuucuggaaaaagaaaauacacuaacaaauacaauaauguuuuauuuacagauggcucuauaguucaacauuuuauaguuuacacaauugccuagaugguaaaaagagcaccuggauguuucugaugcaacuucuuaugacuguuauauucuaagaguagaaaucagaucuucccuauauaaaauuguuacuuaaauauguguuucaaaugaaaaugcuuauuuuugucaaaguagucaauaucaucacgaaaauuuuagaauguccaugcccuuccucuuaaacaguacugaaaagccagugcaagaaagggaaagugauggacugaaggugauauucccaaccuggauagaaagacaaacaagcauccuuccaguguuacaaccucucuuagcuugaauaugggucuccgugcaucacacagaaaaacggcaaagccuugccccaucagcuaauaacaagguugaggguaaaucuggaaagacuugucuugugugacuauugcuuuagagaggagagucuuguuccauguuugcugaaauuaaaucaucagggaagauacuggagcuguucugccucagcaaagcugaguaaauaacgcacucugggacugcucuaaucucacuguuuguguccccauaaaauucauauguugaaauuucacuaaucuaauaccaucacuaaucggugaugguauuagaaaauggugccuuuggaaagugauuagguuaugagggcucugaccucaugaaugagacgagugcccuuaucaaagagaccccagagagcuuuccuguccuuccaccaugugaggacacagcaagaaggugccaucucucagccagagacugagcccuacugggaccuugaucauggacuccccaccuuccagaacuauaagaaguagauuucuguuguuuauaaguguauuaguccgcuuucauucugcugauaaagacauacccgagacugacgcaauuuacaaaagaaagagguuuaaucaauucacaguuccacauggcuggggaggcuucacaaucaugguggaaggugaaagccaugucucauaugagggcagacaagagaagagaacuugugcaggaaaacugcccuuuauaaaaccaucagauaucaugaggcuuauucacuaucaugagaauagcaugggaaagacccgccccuaugguuaaauuaccucccaccggguccuuuccacaacaugugggaauuguaggagcuacaauuuaagaugagauuuggguggggacacagccaaaucauaucaaaaagccaccuaguuuaugguauuuuguuauaggcagcccaaaaggacuaagacaaaggcucucuuugaauuucugucucaaucaucccgaccugcagaaugaagcccucugagaucucuuauugccuugguuccucucugucuuuggauaaagagaguuucuacaaacucaauguuguuugucaccaaagugucugcaucagcuuuccuucuucuacugauagccacuugcaaaaccuccuuaaacuagcuguuuucgagaugguuaacucaaaugccuuguauuaagaagguucaguugaaccugagaugaaaucaagauguuuuuagucacauuucugguaauuauuccacagucuguuaccuuugaauauauaacuucacacggaggucugacaaacugguauuuguuaaggaacaaaucacucccuugcuugauuuauucccuagugagugucauuucuaggauuuguaaaaacaagcuuuaaacagauguaacaaaauaauauuucaugauauuacaaggcugcuauucgcaaagauuacuugcuucuauaucuuuuuucucugcuacugaggcagcauaaauagcuuccuccauguacugcccucagggaaagggugauacaaggagagcuucaaaacaagcucucgaaagucuuaguagaaacaagacucuuuaaagccagaauaaucuaaauucauuagaaagguccgggacuucccagauguguagguuccauuaaccagagucuaaauaaaguauuuagaguagagaguuuauagcacaaaugcuggagaucaggaggaagcaguuaagaccaguggcugcuuucuuacccugaaggccagcgaugggaaggacuucccauccuuggaagagggcuugagggguguggacagagcaagggccaugggcaaugggagggcauggugguuuuacaaaggggcaagucacaaccuggggcugccaugggugcagauaggugucuauugucuucauuccgaaagaaaagaugcuguccauguccccuucucauauccugccccuucccuccccuccacuaucccuccagaauuuacaccacuuucugaaaaggggaauuuucucuccaugcuccaugagagcagccuggaguucuccaacaauguuucccagagugcguuucacagaacacagucuguggaaaacagaccucuuggcucacccugcacauuagccaauuaaagcuuuuauaagaguucugugcuuaacagggucauguccuaguguuuccccacagcacuggaccauagaaauccauuaaacacagacaccucuuucccgggaaucauacugacgcccugugaggcuagcuagcuguugugugaauggcacuuuggagaccaccaggccagguccucggcuaugcggucagugugcccuggguuugccuccuguuccaggucuuccccaaauagggagaauaccacuuaguaucacuggagguaucaggagagacugugccauguaaaaugcagaaacuggcaugucguaagaucugaaaaaugccuugucuacucccuuucccaccucuucucagauaccugagacaaaacaaucgcaaauucccuucacaagcuacuuauuccaucaugcaaggggugagguuauaggcaguuugagaagacugcugcagucccaagagucacaaugauagcagcagcaguucaugagugaugccaguuaaccuuguaagugcuguauuuucaucaaaggcauggaugauaaggacgccauuggguuucacauuuuguccucagauucagagaaagaacuagacuccaggacugaggccuugcugcuugagacucuccagauguaauaauuggaggaaacuagacucuaaggguaaaaagaacaaggggagggaaucugcccucgggugggcaaugggcagcuccagggagaggauuuggaguugagccugagugcugggacugggugcugcccuggcauccccacuccccuccaccaucuucucucccuuugggcacagucaauagggagcaaaacuggccucaaggucaacaugugacuaauaaacauaaaucagaaaagguucaaccucacuaauaaugcaagauauguguaauaauuaaaauaagcugcacuuugggaacaguggugggccccagucugguggcgccugagguuuguacaacuugagggucuccucuaaaguuggagaagaaagagauugaagucuuaccugauuguguuaaaaaugucuaaccuuugcaaaauuuuccguguguguguguguguguguguguguguguguguguguguuuuagauggagucucacucugucgcccaggcuagaguauaguggcaugaucucggcucacugcaaccucugccucccagguuccagugauucuccagccuuggccuccugaguagcugggauuacaggcgugugccaccaugcccaguuaauuuuuguauuuuuaguagagaugggguuuuaccaugugggccaggcuggucucgaacuccugaccucaagugaucugcccgccuuggccucccaaagugcugggguuacaggugugagccaccacgcccagccaccuugcuaaguuuucaaaaaccuaugugaauacauuuccaagaccuuggaaggacccagguuuggggaagggaugagguuggggagcugaaguuuaagucucaguggcuucagaauaaaugcagcuuugcuggggagaguuugaaacagggggagaucugggcaaaaaacgugcuaaaugaguuuuucuuugcucauuugucaucacaacauguaauucucuggguacgucucaagauguaaaugauucggauuucaguuuuauuuuaaauuacccaaauuauuauacuguacuagagaagacagaaaaccugaaggcaaucacuucucaggaucuuuacaaaggaaauauccaagugucacaccuuauuauuuuaauaacaaaauggugauauauuaaaagcaaaucuugauguucaacuuuucaaaguuaugaucccacugcagaaugauaauuaauauucuuuacuuucaggagcacuuuuaggaaaauacagacugagugaggacaggcaaacuuauaugccggguggugaauuuuaaagcaaagaaagccaaugcuauuaagaugaaaauaauugcguaugauuuugaaucuuaaagcauuaccauauguuacuuggaacucccaacauuagaaaaaauguaggaauggaaaaaaagucuguuucauuuuuaaccauuuauuucuugagaugauauuggaccaugcuuuguuuguuuuuauaauuucaacuuuuauuuugcaugcagaggguacauauagaacaugguuuuaaaaugcacaucaauagcagcuucagcuuuucauaauuauaagaagacuuauuuaaaaauucuauaguauuuuugcaguuacaaauuuccagggacuaacggcaaacaaacaacaaauaaaaacaaaagaagcuguaaccaaugagcugcaaaaaccaccaccaccaccaccacaaagacaauuacuguagaagacaaggauaaaaaugaagacuuaagaugacucugaagguugaaggguuaauucagggucacuauuaaauaagaauaugaaaauccauugucacuuaauuaucugguuuaugcaaccugagaacucaagcuauauuaaaaaccuaauaaugcggggcagugaccugggcuucaguagaucaugaaucuuucaccauucaaauuaagggugcguuacuaacacugaucuuuuugcaugcuucacuucagcuauuuaauuaaaaaugccaauagccuaaaggccagauaacuuuucagaaaucaaaaaauguuagcauuuguugcccugucuucaaucuguuucucagauccacugaaguuguuuccuguagguugacaaaugaucuggcaaacacuauguaugaauuauauccagacuggccauauucuggauaucaacuauaucuaagauuucuaccaaauucuguucagccuuuccaacguaacacggcaagucagggagagacuaaaccuaaaaguccuuccuuggcacugaaggucggcuggauccccagaacuuuaaaucuuugggucucccagaguuccugauucauuuucaggugccuuucaccuuguuaccugagccuacagugcagagucaagaagccaucucagaagguguucaauuaauagagaaggcucccagcuggaggggauuuauauguucuauucaguuaaugaaaacaacccuuuaaaagcuauauuugaaaaauucaacuaggguucauucauucauucaacuuaagauugccagauuaaauacaagacacucagugaaaccggaauuucauguaauaaauuauuucuuaguacaagugugucccaaauaaugcaugagauauauuuauacuaaagaaauacuaugucauuuucuuaaaaaucgaauuuaucuggguguuauauaucuuuauuugcuaagucuggcaauucuaacucaaucuuauacuaauaauagccagcaacaauaccuacauagcacugacuacacuuggcacuuaaaauguuugauguguuuuuaauucuugcaacaaccucauaagauagguauuauuuccauuuuucagguaugaugugaaacuggaagcacagaggaaucaaacaaacauugucuuaugaaauucaguaucuuccaccugaaugccuaaagccuggaugguugccuaucuugcagagaugggcagggccaagguuuuccacagaaaacaguucugagcugcuugaacauaaggacaaaugcuugaucaagguccuuaacaauuuuuggcauggaguguuguuguauaauuguagccuggaaaauauucacagcagguguuaccaauugcccucauaaggauauugaauuuuaagaaauuaaauucugggugagucuuccaagauaaacuggcugcaacgcaugccucuuaguagcagccaugguuugaauguuugccucuucccagacucacagugagaucugauugccauuauaaugguauagggagaggagaccuuugaguggugauugggcugugagggcucuguccuccagaauggauuaaugucguuaucauggaguggauucauugguaaguucagaccccuuucucucuuucucucacucugcugccuuucgccauaugaugacauaucaagaaggcccucaccagauacugguaucuugauauugaacuucccagacuaaagaacuuugaaccaauaaauuuguuuaugauaaauuacucauucucaaacauucuguuauagaagaacaaagucaacuaaaacaauagcugaugauuugaccugcuauuuguuugacauacgcuauucaauugagcuccaaugaccccaaaaaugaagauuucuguuuuaacauaauuucauuuauuaugcacuucccaugugucagaucuaggaaaaccaaagauaccuaaggcagagaucgaaaaaaaaccaaaccaaaaaaacaaaacagacaaaaaaaaccacauugagagaauuuaaauguaauauauugaauguuaggauagagauacaggcagaaucuacacaaaggcagagcagagcagggugaugcaucuccaaauuaaggggcuauaaggaugucuuuguggaggaaaugauggcugaacuaaaccguaaggaaugaaaaaguguucauggggcaggaagugcggaaggugauuguaggugaagaaaacagaaaaauggagagaaccuugucucucaucaucauuagguagcuagauccaucauccuuuguguaaauuucucguggaaaaaaaaaaaagccacagguacagcccagaggagaaugucugguucugugcaaugucaggagacuacgagaaggagcacaguuuuacucuguuauuucaagacucuccucauuuuaacauuauuuuugauaguacugaaauagccuaggcauacgacuaggucucaggguauguugcauggagcaaagacaaagucacagaaaauguuggugcuggccaggugcgguggaucaugcguguaaucccagcacuuagggagacugaggugggcagaucacuugaggacaggaguuugggaccagccuggccaacauggugaaaccccgucuuuacugaaaauacaaaaauuagcugggcauaguggcaggugccuguaaucccagcuacccaagaggcugaggcacaagaaucacuagaacccgggagguggagguugcagugagcagagaucacgacacugcacuccagccugggugacagagugagacuuugucucaacaaaauaaaaaaagaaaauguugcugcuaucuuuccucauucuguacuuaaaagauggaauucuacacccuauagacgcuauuuucucgaaguacaaaaguuuuuaaagcuaaauacuuucucugagaguuucucaacuuuagacauuuagcauuagauagggagucuuuaaaauguuuucaaaugugaacucucucuaguauuuucauguuaagcugucgguaaggagugaaaauaaagacuucgaaaagaacuggagcugauguugguuagacaucccaaaauaucacaaaaacguuggcuucaauguauuuaauuuugccccuuaagagaagucuuauucuaggccuggugccuguuaccuagcgaagaaauguuggcuguguccucaaacuuccaacucccaucucauaauuuuaaguucacaaaaaaaucuccuuaaggaagcuuauaggauauacuucaggcacagggcaaugagcgcuagcccgguaaucaaugaaacccggguuugcuccugguuuuguuucuuacuugggcaaaucaaaccguugaguuucuccuuuucuuccucucuaaaauaagacaacugaaguugacacuuagucuaaagaauucuaaugcuauaacuugagcuguuagaugguauuacuuuuaccugguugcauuucaaaucuuuggcaauuuagaaaaaccauaugagaacagggauaguauccagggaacagaugacaaugccuuccacccauccuaauguauaggaauuuagaaauuaagaaacacuuaaaauaaaauuuccauagcuuuauauauuacaacagaagugagaauaauuaguauuucugauacaaacaugacaggucuacuuuauucaaacgcacaggugaguucuuucuguuggcuagaagcuggugaaaaccucuaacaauucuaaauaagcaaauuacaaaguugcucuacuaaugaguuguaagaucuauuugauauuuuacaacaauguauaugguguuuuaaugaauugagaccuuauauuccauauaaaguuuuuuuuuucuuccaaauaaagaugguuuuacuguuuuacuggaccaaguucagauaauauuaacauuccuggacaaauuccaaacuuucuuugguuuuuaguaaucauauaggaauaagagugacuacaacaguucuauuaauacauuuacuucugucagaagccguauuagcaagcauaacaaggaaacauaagaaaauaaacacugaaaauuggacuuuuaaggcauccgucauagcuuauggauuuuauuaacuaaaugccaaaauaauuaaauuacugugaaauuuuuacuaaauguuuauuauuuacuuuggcuuaaaauaaaaaacccauugaguuuauaauaucucauguuccaaaauuaauauauuugauuuacgaaaauaaaaacauaucgcaaugaauuagguucacaucugaaauucacagacuggcuuugacacugcuugugcauuuuggaaacucuucuagcuucucagaaucuugguuucaaaacugaaaagauuggccugacaauuuuaaaacugucuucaagcuaugaaaguuuaugauucaggaaacagcuuucaaaaacuacugagaggacccugaaacucuucugcuagaucgauuccaaauaucuaguguaaaaaacauccaguguuuuccaacaugacucagugggugagacuggguuuuuuaagaccaagcaaaaccgauaaaggaagcuuagaagguuugucuuccaugaaaaagaaaauugcuauugucuuaaauaaacagaagguauguuuuccuuugaauugcacuuagcuuuuuaauagcuaauuucagcuacagaaaaccuuuuuuuuuucuauuuuaauuaaguuuucaaucuggaacuuucuugagaaaggcacuuuagcaacucugaagaacaguagagggcgccacggucacaagcaaagcaaacaaacaaaaacguuggcaucacagacugggccgggaagugccgaggaguacaccugagaaaauaagccacagucagguaggaacaacacauuuucaaaaugcauuugggaaacugccagugaaaaugguccacguuuuucucuuuuuuuuuuuuuuuugagacggagucuugcucuguugcccaggcuggaaugcaguggcgugaucucugcccacugcaaccuccuccucccggguucaagcaauucuccugc

SEQ ID NO: 2 - RNA, 22605 nt, Homo sapiens
auggcucuguguccccacccaaaucucaucuuguagcucccacaguucccacauguugugggagggauccaaugggagauaauugaaucaugggggcgggucuuuccugugcuguucuugugauaguggauaagucucacgagaucugauggguuuuuuuuuugguuuguuuuuuuuuuuuuuuuuuugagacggagucuugcuguugcccaggcuggaaugcaguggcgcgaucuaggcucacugcaggcuccgccccccgggguucacgcgauaucugaugcuuuuaaaaauaagaguuugccugcacaagcucucucuuugccugcugccauccacgugacuuucuccuccgugccuucugucaugauugugaggucuccccagccacauggaacuguaaguccacuaaaccucuucuuuuguaaauugcccagucucagguaugucuuuaucagcagcgugaaaacagacucauacaacuaacaagccugcuuuagcuuuucacuguucugcccaagcaacuuuccccuucagccaggccaguagauuucucauacagcagggugacccuguucagggccuuuguacuguuuauauuuuuuacuuugugcauacuaugaugucuugacguuuuggaaaccucucuggcuggggagagacuguccuucccagagcuggccaguucuuagaaccagaagggcccagccaggagcacagcucugauaugcaaaccaaccaacccagagcuaugccgucuuuaucuagccuguccaccccaggaagcaguauuucuucaccuauaucaugccagagccugauaacaagcaacuagaaaccaugccuguagcuuacagccugccaaaauuauucaaacuagccaauucuaaacuguucacaugcacuccccugcccugucccugaaagcucaagaaagaccauggcccaggcucuccccucacuccugccccuucaggcuccuacaggggggaccaagacugaugcuuccccauguggcccugcagggcaugacaugcuucccgucucuaagaccugugaguauaauaaccuuggccuuccugagcaccuccucugccuccuccuguggccacaucucacugaccaucacaaaaaacaacauagaauaagccaggcaugguggcuuaugccuguaaucccagcacuuugggaggccgaggcaggcggaucacuugacgucaggaguucgagaccagccuggccaacaugguacaacccugucucuacuaaaaacacaaaaauuagccaggugugguggugggugccuguaaucccagcuacucaggaggcugaagcaggauaaucacuugaacccgggaggcagagguugcagugagccgagauugugccacugcacuccagccugggcaacagagcaagacuccgucucaaaaaauaaaauaaaacagguuuucauuuuaauuuuuaacauauaaaccuccacaggaacauucugacacagacuucagccccaggccuacaucacccuaugauccaaauuauuuuauaguaaaugguaugaaguuuggagaggaagggcaauuuggccaucaugcugugauuuuacugcagguaaaguagaaucaccaugaaagugguggagccuugcuuuuucuuuaauuuuuauaaugccaccacccuacauucuuaggagaaaaggcaaggaguuaaauaaauaagcacagauuuaaguucacauuucugguaauuauccgcaagcuaauaacugagauugcuuaagcucuguaaacaaaacacacaaagcccuuguggcucugcgccagaaucaaaggucugauccaaaacucccccauccccuuguuggcuuugaacaugaaaccaauuuucuacaccaaaucaggccuacuucuuuguaaccauccuuaccaucucucuuauaggaaaauguuuaggacucuuggcggugacucuagaacuaggaaaucaacucucucagcagcacucuaaugagcaaaaacaacugugccucaaaauacaggcugcagugggcagaaaucucuguacugcacuccagccugggugacagaguaaguuucugucucaaaaaaaaaaaaaaaucaaguaacaaagaacagcugucuaaaacuguuaacacugauucaaaauauaauaaccuuuaugccuguuauucagaguuaaaguggguacgcuaauuauaaaucauacacuuuucuaauugugcaauacucugucaaugggauagauuuuaggaaaauuccagcugaaccaguccauucuuugaaucauuaggucaauuacaaacaugccaauccuuagccugaauagcugcauauuuagaaagaccuaaaacauucuucaaaaagcugccauacauggucuguuaaagggcuuacuugguaggaaauaaaucaagagggaauacuccauagugaaucucagggcacuuuuuaaaacaaaucucagggcugagcacaaugguucaugccuauaaucucacugcuuugaggggccaaggcaggaggauugcucaagcccaggaguuugagaccagccugggcaacauggcaagaccuagucccuacaaaaaaauuguuuuauuagcuaggcaugguggaacacgccuguggucccagcuauuccagaggcugaagugggaggaucacuuaagcccaggaagucaaggcugcagugagccaugaucucaccacugcacucgagccugggagacaguacaggacccaucuccaaaaaaaaaaaaaaaaaaagugaacgaagcugcaauaagcugugaucauaccacugcacacuagccuaggcgacagagugagacccugccucaaaaaaaaaaaaagaaaaaaagaaaaaaccaauugauaaaagaguaaccucccaauccccagcuguggaacccaagagcagagaaggcagccacccgccucucuuucccucaaagacaccagagugcaauaggcucuuccucacucauucccaugggaaguagccuucaaccccucucaaaccacagcauauagagaugccuuuauuaauaaaaaaggauacaacccacgggccagccgcaguggcucacgccuguaaucccaacacuuugggaagccaaggcaggcagaucaccugaggccaggaguccgagaccagccuggccaacauggugaaacucugucucuacuaaaagcacaaacauuaaccgagugugguggcacacgccuguagucccagcuacuuaggaggcugaggcaggagaaucgcuugaauccgggaggcaaaaguuggaguuagccgaagucgcaccacugcacuccagacugggcgacagagcgagacucagucucaaaaauaaauaaauacaauaaauaaauaaacaaaguauacaacccacaacucacuuuuuuuucuaaccaggaaucucaacaacaggacuauagggaaacaagaauuauuaaccucauuaauucaaauuaacucuuaagcacccaccacuaacaggcacucuuauagguucuagaccuaggaucauuuuuacagaguucacauuggggaagggagaggggagagggaaaggcaaucacacuaaaaagagggggucagaguaacaguaagauccacacagaaggccaggcacaguggcucacaccuauaauccaagcacuuugggaggccaaggaggguggaucaccugaguucaggaguucgagaccagccuggccaacauggugaaaccuugucucuaccaaaaauacaaaaauuagccgggcaugguggcgggcgccuguaaucucaguuauuugggagccugaggcaggaggaucgccugaacccaggagguggagguugcagugagcugggaccaugccauugcacuccagccuaggcaacaagagugagacuucucaaaaaaaaaaaaaaaauccacaaagaaaguagggagccaugguagugaaugggggcguuuacucuagauggguggucaaggcagaguuucccaggaggacaaagcccccagugacaagagcagggucaacaaguuccaagcaaagagaacagcaagugcugaagccuaaggcuaaugcacuuacagaaaaauccuuaaugacuaaguguguugccucuguuugcgcagagaucacaaaacgugucccgcagaagggagagaaaaagggaccccaggagaggguggcggggcuucagguccuggagaaaggcagcaagcauggcuaacacauuccuugguccacccugcaggguaauacugcaccuucaauugacuuaugaagaaacuagcucagauuggaacaacagccaggacagucaccaccaaggggacuugccccacuggagaaggcaagucugacccccuaccccaccgcggucaacccagggacggaggagagaacgccgcaugccauuguucuuauguauaaagcacagguuacagguuauuucugaagaugucugcucaaguggagaugcuaccagguuuuuaccaaucauuacuuuggauaaaaguuuagcaaauauuuacaucacugugcccacaguuugugcuuaugccccauggcuuuagagggcccguauaccuuauguauacuucauauuauguauaauaugaacuucagcuccauucauggucauaucaucuaaccugggauacucuuaggagugaaagggggagcuuaauaguggcagugugauaagggauauaaaccaggauucuccugggccaacugguacaggugaaaaggaugacugaaacggcagagguccuccauucuucuagaugggugaucaaggcagagguucccaggaggaccaagcccagacagagaggcaaggggcgguggcucacgccuauaaucccaacacauugggaggcugagguggguggauugcaugagccagggaguuugaaagcagccugugcaacaugguaaaacccugccucuacaaaacaaaaacaaaaauuagccaggcaugguggugcaugccuauagucccagcuacucaggaagcugaggugggaggaucaccugagcccgagcacagggagguugaggcugcagugagcuaugaucgcaccacuuauacuccagccugagagacagagcaagacccugucucggaaaaaaaaaaaaaaaaaaaaaaaaaaacacagacugacaacaacucaaugaacucagauaauuagaaauucuauuagucuaauuucauaugaaaccaagguauuuuuuuuuuccuuccucagagaccggagugggagagaaacuguuacagccaaucacuuccuuccaggaaaaaaaaagacgcaaauaacaacacaaaaaaacgugaucuaauagacaauccagauauaaagaacacaaacccuuuucccuacaauuuaaccaaaugcuaaaaugcaccaggaaacacaggauacuacagagauaaagauuuggccagcagauuaucaaccccaggaaaagaaauccauuucuguagcaugcuaugcuguuggaggcacacgucacacuucuuaauucaaggacagaaaagcacaaacuaaaaugcacacuggaguaugacucccccuucucuaucugcuaaagcgcuucgcaugugacuuucaaucucuaagcugggaaucaaucaggcagcccagccuccuauauuuucuaguuguauugcuuugagcaucaaauuuaaccucugugcaccucaauuucuucagcuggagacaaagaaaguggcugcacuguggcagaguguuugcaaauguuaaucaugguuaugguagcauucuaccuugucuuacagaugguugccuaaaaagguaggaaacuguucacgaaaaagucucaaccgguccuagagugaucagaaugacagcgcugaaauaaggagggcaguaccucuuauuccagagaaguggggagcagagaggaagauggaguggaaaggggcgaggcaaggcccuccugaaauaccucaacccaaaucuucaagaaauccccaaguccucacagugcuuuuuguggauuuuuguggaaaccgguaaaaggggcugauuugcugaccccaguggguagaaaacagagacugucaagagaacagaagagaaggcagaaaggggauggggaagugggguucgccauguucacgagcuccuggagccacagggccccccaggaacaacagagcugagacuggguggccuuguuucuggcccaauucccugggaccaccacccuggaaggacacuugagcccuacucaaggacccaccacaacaucucagcauugucgguccuuaaaugccaagguuugugcucaccauuuccauccuuaagauucucacagggaagaacccacacaacagcuuugucccugcgacucugggugaguuucugacacuggccugggcucccguuuugcccgucaccaacuucucacaguauaacuauccaagagaaguagcauuugaugucagggaaucaaaagggcuaaggcucgggaggccaaggcaggugaaucacuuguaaucaggaguucgagaccacccugaccaacauagugaaaccccaucucuacuaacaauacaaaaaauuaaccaggugugguggcaggugccuguaaucccagcuacucaggaggcugagguaggaggaucacuugaacccaggaggcagagguugugguacgccaagaucaugacacugcacuccagccugggugacagagugagacuccaucucaaaaaaaaaaaaaaaaaaggccuaaggcuuacucagggucaucuaugguuugccaagcagaguucaauccaaucaacaaaacccucacugcccacuuuuccaaaaccugauauagucagucacuaguagcugccauuugcuguuuugccuucuauuaaaaaaaaaaaaaaaaaaaaaaccauccaauucccagaagucaaaggcaauuccuucccuuuucugugucugugagacggcucucugugggauuccuuccugcugcaccucauccuuccuggaagggacucucagaaaaugugccuccuccuuacaggaaaggaauuccucccgaggaacuguggacaugaggcagcuuuguggaauuucccuuccacagacuucagccuauuuucugggcaaacuuagcagaaaugaccaaaauccacucgagagagacagcaacaaggagaggggaagagauccuauuuuggcuucuucaaauggaccaauuuucucuuuaaaauauuuauuuuaaggaaaaacaaggacuacauuauauuuuaaagggggagaaggaagaggauuuugaguugcuaagagaacagagccaucucuuuucuuuuaauacuggaugcuaagaacuugcacaggguuuucccaaaagccagauaguucagguaauuugcauguggcuguaaaagagaaaguucaggccaggcacuguggcaacauggugaaaccccaucucuacuaaaaauacaaaaauuagcugggugugguggugcaagccuguaaucccagcuacuugggaggcugaggcaggggaaucacuugaaucugggagguggacguugcagugagccaagaucacaccacugcacuccugggcgacagagacucucucaaaaaaaaaaaaaaaaaaaaaaaaagagagagagaaaguugagagcauaagucugagugauucugccugaaagaacagaacagcuuacacuggacaggcuuuugcccaagacacaccucaucuggcauugagaaacgaacaccaacaauaaugagugacagaauguuaagaacaauaauuauacagacuuggaccucccagacugcuugucuguaguaauucagaaggaagcaaaguauucugaaguguuuaugaagauugguuccacaucugaagaauuuuaguaaucuuacccaaacucaacccaagaaauaugaggaaaagagggaguaguucuauaauaagcagguggaauagagggucaaacuccuuggcgaguucagucuaacagaggacacauuacugcccuacaaagcagcucuggaaaaugacaaccaguggcacuaacuccaggcaauaucuuugugaggcuugugggcuguaauauagucugcucaggcugccauaaaaaaaaaaucacaaucaggccaggcacuauggcucacaccuguaaucccagcacuuugggaggccaaggagggcagaucaccugagguuaggaguucgagaccaggcuggccaacauggcaaaaccccgucucuacuaaaaauacaaaaauuacccggaugugguagcacgcaccgguagucccagcuacucgucaggcugaggcaggagaauugcuugaaccugggagggguggagguugcagugggcugagaucacaccacuguacuccagccuggacaacagagccacugcacuccagccugggcaacagagagagacuccaucuuaaaaaaaaaaaaaaaaaauccccgucuucaaauacaaucacauucugaaguacuagagaucaggacuccaacaugcgaacuuuggggggacauaauucagcccguaauaugggugauaaaggagcagguccagacuuagagaaacaaggacucccugggucaagcucuggaagugaaaggcuucuucaaagacaggaaagucccacugaagguggcuuuggaagugcacccuugcagcacagcaaccuucuugauuugcucaagagcagggguucugacaggucucaaagcacuuuguuuaaugaaacagaaucgaaaauaccagugugucacauguuuccuaagacaggacacuguuucuugaaacuuuuacuaaaguauguuuuucuuuuauuuuuuuauuuuuuauuuuuuuugagaaggagucuugcucugucgcccaggcuggagugcaguggcacaaucucgacucacugcaaccucugccucccggguucaaacaauucuccugccucagccuccugaguagcugggauuacaggugcacgccaucaugccuggcuaauuuuuguauuuuuaguagagacaggguuucaccauguugaccagucugcucucaaacuccugaccuugugaucugcccaccuuggccucccaaagugcugggauuacaggugugagccaccguguccagccuaaaguauguuaaauauguuaugugugagagagagaaagagaauauaucucuuggucauuacauauguaaaacuuguagaacgugaucaaaauguuuaaaaacuggccaggcacaguggcucaagccuguaucccagcacuuugggaucccaaggcagguggaucacaaggucaggaaucaagaccauccuggucaacacagugaaaccccaucucuacuaaaaauacaaaaaauuagccuggcgcgguggcacgcaccuguagucccagcuacuugggaggcuggggcaggagaaucgcuugaaccugggaggcggaggcugcagugagcugagauagcgccacugcacuccugccugggcaacagagcaagacuccaacucaaaaaaaaaaaaaaaaguuuaaaaacugguccuguggcagugggucucaaacaucagcaugcaucagaaucacuuggaggacugauuaaaacacaggcugcugcugaugcugcuaauccuagagccaccuugaaguuuuucugugguuuuuuuuuuuuuuuuuuuuuuuuuuuugagacagggucuugcugucucccaggcuggagugcaguggugcaaugauaccucacugcagccuugaacuccugggcucaagcaaugcccccaucucagccucccaagcaacuaggacuguagcuagcuccuaacucauucccuccauuccugcccuccccuaacuuuauauugcagccagaaggauauuuuuucugaagagcaucucaugcugaguggcagcagaaugcagagauuaagagagggggccaggcacaguggcucacaccucuaaucccagcacuuugggaggccgagguggguggaucacccgaggucaggaguucaagaccagccuagccaacauggagaaaccccgucucuaugggcgugguggugcauaccuguaaucccagcuacuagggaggcugaggcaggagaaucacuugaacccaggaggcagagguugccgugagccgagaucucgucauugcacuccagccugggcaacaagagcgaaacuccgucucaaaaaaaaaaaaagaguggcgccaggugcaguggcucacgccuguaaucccagcacuuugggaggccgaggcaggcaaaucaucugaggucgggaguucgagaccagccugaccaacauagagaaaccccgucucuacuaaaaauacaaaaaaauuagccgagcgugguggcaugugucguaugccuguaaucccagcuacuggggaggcugaggcaggagaauuacuugaaccugggaggcagguuuuuuguuuuuuguuuugguuuuguuugagauuuugguuguuguuuuuugggguuuuuuugagacagggucucacuguguuguccaggcuggagugcaggggugcgaucaugugucacugcagccacaaccuccugggcucaggugauccucccaccucagccuuccugaguaacuguuacuacaggugugcaccaccaugacuggcuaauuuuuguauuuuuuguagagacaagaguuucaccauguugcacaggcuguucacaaacuccuaggcucaagcuauccaccugccucagccucccaaagugcugggauuauaggcaugagccaccuugcccagccaacugagccacuuugugccaggacuaggucagguacuugggacauauacaggcaaggacacugcccacagggaacugacagugucuagcagcuacacagaugacacggccuuuucggcagacaccccuguccccugugcuuggcaaacagcagagacucaaaaauguauaugguuugacucccacuggaacauaagccccuuagggcagggaccagggucacuguauucccagcaccuauaaucaagagguacaugacgcugcagcugagagcagggacucugugcuaaguuaccuaccugguuugaaccuaggcucugccacucaaaggcugugugaccgugggaaauaauaguacccaccccaaagguuuguuauuaguauuuaauaauauuuauugguaagguacuuagaauagccccccugccuacaggaagcaguacauaauuauaaugucuaaauauacaguaguuccaggccaggcaugguggcucaugucuguauuccuagcagaaugagaggccgaggaaggagaaucauuugaguccaggaguuugagaccagccuggauaacauagugagaucucgucucuacaaaaaauaaacaaaaguaagccagacaugguggcacaugccugcaaucccagcuacucaaaaggcugugauuagagaaucacuugagcccaggagguagaggcugcaaugagcugagaucacaccacuguacuccagccugggcaacagagugagacuccgucucuuaaauaaauaaauaaauaggggccaggcgugguagcuuacaccuguaaucccagcacauugggaggcugaggcgggaggaucacuugaggacaggaguucaagaccaaccugaccaacauggugaaaccccaucucuacuaaaacacaacaaucagcugggcaugguggcgggugccuguaaucccagcuauuuugggggcugaggcaggagaaucacuugaacccaggaggcagagguugcagugagccaagaucacacuacuacacuccagccugggcaacagagcgggacuccaucucaaauaaauaaauaaauagcaguuccauauccucugaauaauggacuagaaguugguuaauggauggguuuucagggagaauuuuagcagccgagcugagagguagguacucuagccaggguauauaacaugagcuaagggaacaggacagcaaauauaaggugugaucaggggaaaggcaucauauauugacuguccaggaacaauggauaucugagaauguaggaaagcuaaguuaggaccaggaaaaggagaguauuaaaugccaaagaagaugccaccaaagguucuggaacagaaagcagcagagcuaugccuuguaaagauaaagccugcaguguauugacuggguuggaaucuggaaucauugcgugccuccagucaagacguggcaacugaccaacuaccuuuuaucacugaccagacucuucccuacgugcagauaacugaggacgcaagccuuaguuacugggugugguggugcuauuaacggaaaaagggaagucagccagcaaaggcugacuggggcaauuucagccuuguugcucugcugugaaacugcuuuccagaaaaacuuuuuuuuuuuuuugagacggagucucacucugucacccaggcuggagugcaguggcacaauuucggcucacugcaaccuccgccucccagguucaagcgauucuccugucucagccuccccagaagcugggauuacaggcgcccaccaccacacucagcuaauuuuuguauuuuuaguaaagucaggguuccacuauguuggccaggcugaucuccaacuccugaccucaggugauccgccugccucggccucccaaagugcugagauuacaggcaugagccacagugcccagcccaggaaaacuucuuaaagauguaauaauaacgcaccuuauuauuuuccaaguacuuccauagauaguggcccacuucugaguaugaauuaaauuucccuuuguggccuuuguuuuuuuaauagaucuucauuucuggggggacuuacaaacaggugacuucuccauucaccagagaugggacauuauacacugaccuacuuccauauucacuuucccugcuugauuggaggugagggagaaaaauuaguguauaaggccgggcgcggaggcucacgccuguaaucccaggacuuuagaaggcugaggcagguggaucaccugaggucaggaguucgagaccagccuggccaacauggugaaaccccuugucuacuaaaaauacaaaaauuagccaggugugguggcacacgccuguaaucccaguuacucaggaggcugaggcaggagaauugcuugaaccccgaggcagagguugcagugagcugaguccaugccauugcacucuagccugggcaacaagagugaaacucugucucaaaaaaaaaaaaaaaagaaaagaaaagaaaaauaaguguauagaacuuuauuguuucauugaccgggcacgauggcucacgccuguaacucuagcacuuugggaggccaaggugggcagauuggcugagcucagcaguuugaaaccagccugggcaacauggugaaaccuaucucuaauaaaauacaaaaaauuagcccgguaugguagcacaugccuauaguuucagcuaccugggaggcugaggcaugagaaucacuugaacccagaagguugcagugagccaagauugugccacugcacuccagccugggcaacagagcaaaacucugucuccaaaaaaagagaacuggccagggaugguggcucacgccucuaaucccagcacuuugggaggcuaaggugggcagaucacgaggucaggaguucaagaccagccuggccaacauggugaaaccccgucucuacuaaaaauacaaaaauuagcugggcaugguggcaggcaccuguaaucccagcuacucaggaggcugagacagcagaaucguuugaaucugggagguagaaguugcagugagccaagaucacgccauugcacuccagccuggguggaagggcaugacuucgucucaaaaaaaaaaaaacuuuuuccuuaaaauacaccucuuggagccagucccagauacuugggaggaucaggaaggaggaccucuugaggccauaaauuugaggauguagugugccugugaauagccacuacacucuagccugggcaacacagaaagaccucauaaauaaauaaauaggccagaugagguggcucacaccuguaaucucagcacuuugggaggccaaggcuggcagaucacgaaguuaggagaucaagaccauccuggcuaacacagugaaaccccgucucuacuaaaacuacaaaaaauuagccaggcaugguggcaugcaccuauagucccagcuacucgggaggcugaggcacgagaaucacuugaacccgggagacggagguugcagugagccgaaaucgcaccacugcacuccagccugggcaacggagugagacucugccucacacaaauaauaaauaaauaaauaaauauuuuauaauguauuuuuaaaauauaguucuuaaaacaugccaugaguagguuuaauuuuucuaaaucauagaauaggauacugaagcccagaguggucaugugucuugccuaaggucacacacagcuaauauauacaagaucaugggucuucuggcuccagucugaggcugucacaauaacuagcuucucacuugguccuaccuaagacacaggugucagcaacucuaaauucaagaaauugagauuaagacuaaaguaauagcuuauaaacauuaauuaagcauauuaauugcaagauguuaaaccagucacugagagauuaagacauugaagauacugguuucugccuuaauagagcuaacaauuuaguaaaaaagucgcaaaugagucaagacuuaaauaagacuuaaagacuuaagacaauaaaaaaggcaugauaaaaggguaaauuuaggcuggacgcaguggcucaugccuguaaucccagcacuuugggaggcugaggugggcggaucaccugaggucaggaguucaagaccagccuggccaacauggcaaagucccaucucuacuaaaaauacaaaaauuagccaggugucauggcacaagccuguaauuccagcuacucgggaggcugaggcaggagaacugcuugaacccagaagguggagguugcucaagagcaagacucugacucaaaaaaaaaaggaugggggauaaauuuagcaagaacccucaucaucccauuuaccuacagccuaauauguacuaggaacuuacauauacaauaauagguaaaaucuauuaagaauguauuauguuuuauaguugaacaaauugugucgcuuaaucaacuuagaauucauguaaaugccacauuugagcucuguuacauugagagcucuccaggcccagccuaccauguuaagccauugauaaaccucccugcuacuauuuaaaacaagauggauaccuuucuacugucuuuaaagaggaagaguacacacuguuggacuguaugcacucuccgucucucaagacaguaaucugagggauuacuucaauagcagaaguuaaaagaagcggucaguuggguuuauuacauaaucucaaucacauccugaccaauaaaaugcaccaauaaagaucagcaaauaaaagcaagugauuagaagagcaauuuuaugcucacgagcucugaauucacaagugugagauuuccugucaaguacucucaugagcucaauuaaucucugacccagggugagaaaauaaacauagugacgcagaggcugcagcucaugcuuuggaauuugucagacuugacucaguucccaacuccaucauguccugccugggugacccucauuaugcuaccuggacucucucaguuuccuucaccuguauguaacacaagagcagagccacccucaacaagggguuacuauaaaaaucgaagaacccggcaucucuaacaacaucccuggcacggagcaaucccccuucucccucagccacuggcugagcacccacagaggggaguaaguccuauugugguaaugguucucaugcuuugagaucagacaaguccuuugagaggaaccugaaaaaggggcagcgaaccuuuggccagaaaaauggaaauaugcacagaaauauuuuugacuuuggcaagcaguugaucagugguucuugggcuucagugcccaucagaaacguagagaaccuuuuuaaaauauugaugugcggggcgcgguggcucacaccuguaaucccagcauuuugggaggccaaggcugacggaucaccugaggucgagaguucgagaccagccuggccaacauggagaaaccccaucucuacuaaaaauacaaaauuagccaggcaugguagcgcaugccuauaaucccagcuacucaggaggcuaaggcaggagaacugcuugaacccgggaggcagagguugcggugagccaagaucacaccauuccacuccagccugggcaacaagagcaaaacucugucucaaaaaaaauagauagauagauagauagauagauagauagauagauagauagaugauagauagauagaugcguggaucccacccccagaaauuauuuuuccaauuggccuauaaggggaacagacauccauauuuuuauggcuguccaggugauccaaucauggugagggcuaagaaacugcuauguaaucuaaaaagaauuuagaaaaaauaaagcuaauaagguuaaaagaaauaaugcacaucaacaaucauuugaagggagcaauuugagaguauauucaugaaaagcagcaccaagagccagcuuguguuaagaggaaucauggccggguguggugucucacgccuguaaucccagcacuuugggaggccaagguagguggaucaccugaggucaggcguucgagacuagcauggccaacauggugaaacccuaucucuacuaaaaauacaaaaaucagcuaggcguagugccacaugccuauaaucccagcuacuggggaggcugaggcaggagaaucacuugaaccugggaggcagagguugcaguuagccaagaucgugccauugcacuccagccugggcaacaagagcaaaacucugucucaaaaaaaaaaaaaaaaaaaaaaaaaagaggaaaucgugaggccaggcacaguagcaucgcgccauugcacuccagccugggcaaaaagagcaaaacuccaucucaaaaaaauaaauaaaaauaaaaaauacaaauacaaaaauuagccgggugugguggcaagcaccuguaaucccagcuacucgggaggcugagacaggagaaccgcuugaaccccgggaggcagagguuguacugagcugagguugcgccacugcacuucggccugggcgacagagcaagacccugucucaaaaaaaaaaaaagaggaaaccaugccaaaugacuuuaacccuuuucuugguaggguuucugaaugacugcccaggcugggcaugacgguucacaccugucaucccagcauuuugggaggccaaggcagguggaucacuugagcucaagaguucgagacuagucugggcaacauggcaaaaccccaccucuacaaaaaauuagaaaaauuagccagcugugguggcccgcaccuguagucccagcuacuuggggggcugaagcaguaggaucacuugagccugggaagucaaggcuccaauaagcuaugaaugcaccacugcacuccaaggcuggcuggcaaggcaagauccugucucaaaaaaaaaaaaaagacagaagugcccaggugauccaauacuccaauaccucuugacuuccauaaagcagaccuccauaugacacgaccucucuugacacuuguggccaaguuaaucuagcaaccacaguguugauuaguggaucugccacaaccuagaggacauuccuaggacccauccuuucuagcauucagacuacgaaaguguuccaagaacuuucaacagcaacaaugagcugcuaaacguuagcaucuacaaaaugaauuugcauagggauagaagagcaguuacauggggacaagcccugcggacugugaggucaauuggccauguguccaauauagcucaacugcauguaucacugaugaugcccaaauugacauaaacuuccugaccacauuggccaacaugaugaaacuccaucucuacuaaaauacaaguauuagcugggugugguggcgcgugccuguaaucccagcuacucgggaggcugaggcacgagaauugcuugaaccagaagguggagguugcagugagccaagaucauaccacugcacgccagccugggcaacacagugaaacuccauuuaaagaaauaauaauaauaagauaaacuucuccaugagccccaaacucaugaccaauugaaugccuauucaacaucuccacuugaauguguagcaggcaucucaaacuuaacagggucaaaaauaaacuccuguccaggcaccauggcgcaugccuguaaucccagcacuuuuggaggcuagggcgggaggaucacuugagcccaggaguucaauaccagccagggcaacauggcaaaacuucaucucuacaaaaaauauaaaaaauuagcugggcaugcuggcacacaccuguggucccagcuacucagggggcugagguggaaggauggcuugagcccaggaggugaaaguugcagugaacugagaucgcaccacugcaauccagccugggugacagagugacacccugucucaaacaaagaaacaaagauuuaauguggccaugauagcugucuuugaauacuaugaggcgagcauaugaagggagguagccaacuugcucugcguguugacaaaaaaacaaaggagagcggcucugaaccuauuuugguucuaggagcugcuugauucacagaucauucuuugcuucauuaaauucuguaaugcuuuucuuagaaguaaguaaacacaaguagaaauugcauggaaagaaaacuguggcucaauacaacauaauucuggcauucgaaucuaaagucaugagagucacauaaugcauuuccccacugcccaguaucuggaagggauuccuguacugagugugggcuagaagaucucuuccuuucaacccacuaauucuaugacacuaaggaaaaggggaagauaggagcuagcgcagagaaaagagcagacagggacuaggccugaaacagaccagcacggaaguggucauggaacccagaaauggaacaaggagcuacgucgcaggccacaaaagggcuguaaguacucaugaguaaggggcuggaagugggagaagcaaagucugaacaucugcugggaugaaagaggggagggcaaccuuggguguaacaaacgcuaccggcuggcagggacgacauagcgacugagggguggucauucgccacacgucucaggcaacacuuucuccaacugcaguucugugaccuggacaucucauccccugccaggaacgauuucccaccuucuccauccaagcuccaacaggaagguaaaccccuccccagagaucuccaucuuggccaacauggcaaaacccugucucuaacaacaacaaaaaaauuagccaggccugguggugcacaucuguaaucccagcuacuugggaggcugcggcaggagaaucgcuugagcccgggaggcggagguuguagugagccgagaucaugccacugcacuccagccugugcgacagagacuccaucucaaaaaacaaaaacaaaaacaaaauccccacccucacacagcuuuuccauacuucuacuccagcacacccaacacggguucaggaugaacuccugcagcagcgacugcuggacacacggacuauguuuuuuugagauggagucucgcucuguugcccaggcuggagugcaguggcgugaucuuggcucacugcaacuucugccucccggguucaagcaauucuccugccucagccucucgaguagcugggacuacaggugcaugccaccacgcccagcuaauuuuuguauuuuuaguagagacgggguuucauuacguuggccaggcuggucuugaacuccugaccuugugauccaccugccucggccucccaaagugcugggauuacaggcauaagccaccgcacccagccuggacuacguuuucuuuugcuuuuuuuuuuuuuugagacagagucucauucuguagcccaggcuggagugcaguggugcaaucuuggcucacugcagccuccaccuccuggguuaaagcgauucuccugcucggucucccaaguagcugggauuacaggcaugugccaccaugaaagaaggcagggugccuucuuucuugccaggugcaaauccauuacaauaaaguaccauuccuguucucaaagagcucacacaaugagggaggcagccacuguaucgugcaaauaaugcuagcuauuaaacaagcccugauucucaauggcuuaauucaaaaucaccagccgaggcaguggaucacaugaagucaggaguuggagaccagccuggccaacacagcaaaaccccuucucuacuaaaaauacaaaauaaauaaauaaauaaaaaauuauccgggcgugguuuugagcuccuguaaucccagauacuugggaggaggcuaaggcaagagaaucgcuugaacccgggaggcagagguugcagugagccgagaucacgccacugcacucuagccugcaagacagagauagacuccgucucaaaaaagaaaacaagaaaccaagaccaugccuauugucaugcuauaguccccuuuugggccaucuaggguggugaacaacaccauacauuuacuccuagcuggacuuucaccaucuugcggcucggccuccucucaguccuccuccucuccaggaggccucuuuauucagcuggagaggcguugcauggggcugguuuuuguguuccagucguggaagcgagacaccuuuugcaaccacuuccuauuggccaggucucaguuucauggucaccucugaccacaagguugggguagaaaacauaguccaggcugggcauggcggcucacgccuguaaucccagcacuuugggaggcccagguggguggaucacccgaggucaggaguucaagaccagccuggacaacauguugaaaccccaucucuccuaaaaguacaaaaaauuagcugagcaugguggcgcacaucuguagucccagcuauuugggaggcugagacaggagaaucgcuugaacuccggaggcagagguugcaaugagccgagaucacgccacugcacuccagcuugggcaacaagagugaaacuccaucucaaaaauuaaaaaauuaaaaacaaaaaauaaaauaaaaauaaggcacucaaauguuugcuaaaugaauaaucuaaaccgcagaggaucaaggcaguaaccuuggccucccagacaccgaucaaguagaauuggacaccaaagacugagaaagaccugagaaacguggcucccaaaucucagcaucugaaucaaccaggaaauaaugcuuuaaaaaaugcagauuucaggaugcucaaccccaagagacugugauucugcagacacagaaauuuacuauuuggccggccacagugacucacaccugcaaugucagcacuuugggaggccaaggcgggagaaucacuugaagccaagaguuuaagaccaacaugggcaacacagcgagaccuggucucuaccaaaaauguauuaauaaaaauuacuggccaggcacaguggcucgcccccguaaucccagcacuuugggaggccgagguggguagaucacaaggucagaagaucaagaccauccuggucaacaugaugaaaucccaucucuacugaaaauacaaaaauuagcuggaugcaguggcgugcgcuuguagucccagcuacucaggaggcugagacaggagaaucacuugaacccuggaggcagagauuguagugacccaagaucacgccaccgcacuccagacugggcaacagagcaagacuccgucccccgccccucaaaaaauuagcuagguguccaggugcgguggcucaugcuuguaaucccagcacuuugggaggccaaggagggcggaucacaaggucaggaguucaagaccagccuggccaacauacugaaaccccauuucuacuaaaaacacacaaaaaaauuaguugggcauaguggcaggcaccuguagucccagcuacuugggaggcugaggcaggagaaucacuugaacccaggaggcagagguugcagugagcugagaucacaccacugcacuccagccugggugacagugagacuccgucaaaaaaaaaaaaaaaaaacacuuuagcuaggugugguggcgugcaccuguagcucagcuacuugggaggcugagcuggaaggaucacuuaagccuaggaauuugaggcuacaaugagccaugauugcaucacugcacucccaccugggcaacagagcaagacccugucucaaaacacacacacacacacacacacacacacacacacacacacacacacacaaaaacacaaaauuaaauggccaggaauaguggcucauuccuguaaucccaacgcuugggaaaaccaag

SEQ ID NO: 3 - RNA, 38666 nt, Homo sapiens
cccccauuaaugcagccaaccuucucucugggccacuagacagucuuaaacauacugaacuacuugucuuuccuacccgaagccaaauucucucaguuuuugcauauguugcucccucuccaaaucggccucuuccuaaaaauguucaagggucguugacuacaugcuauuuuguuuuaggcacaaacauuaaaauauuguuccugcauaaaauaggacuucacuauaaacaaauacauauuuuaccucaagauuguauccucagaacuuuaagucuuuuucuucccuuuccacugauccacaccuccaugccucugaacugggacucauaguuugauuugacucuucuuugcaccuuuuugaugauuucuauauuauauacugguguuuugcauccgagcuaauuuguccacaaugacaaaaugggcgaucccacauuccuaauauuuccucugcauuccacugcuugguuccuuauaugcaguaaauuaucacauuuuuuuuagaugggggucuagcucugucacccagguuggagugcaguggcacaauccuggcucacugcaaccuccgccucccaggcucaagagaucuucccaucucagccucccgagcagcugggaccauaggcgugcaccaccaugcccagccaacuucuuuuuuuuuuuuaauuuguggagaugggggucccgcuauguugcccaggcuggucucaaaccccugaccuccagcaauccuccugccuuaacucccacacuauugggauuacaggugugagccacagugccuagcccucauacuuuugagaaaaaaaaauuccuaucauggggcuuuacaacuguuauuauuuuuuaauuuccaaaaauaaauaauguauuacuaguuaugguacugcucauguucacgaucuuaauucugaagagaguuaaaugcuucaagaagaguggagcucccuucuccuuagcaacaugcuuauuuucuaauagcagccuuuggcagacagugcucccaccuuuucaaugacuguugugggaauuucaaguuuggcaggaggacagaacaauauccuggcagaugaauucugcauccuucagaucguaugauguccuaaauaagacaacugacuucucuacccacucacuuucucggggacauuuacugagggccuauaaacugugccaggcauuguuuagaugcuguaaaacagaucaguaugacuuggucccuguccucaaaggaaucuaaaaacagucguaaaagacaccuauguaaacaauuaaaaugaacaggaugaaugcuauaacaaagacauuauugaaauuuuguaggaguauugaaaaagaacagguaacaagagggcucaagaaagcuucacaaagggggaacaagcuggucuuaaaagguaaagagaaguuugucaggaguagaagggauggaaaagcauuucaaauaaaaggagcagcuuguacaaagcagaaaagugugugccaaggaaugauaugaacucaugaacuucagugugaguggacuauagggaacaguguuauaaaaugaagcuggagaagaagguuggccugagauugcaaggggaccaaaaauucuguuaagaagcuaagcauuuugaccagcagcagacagccccaaacauuuucaagcagggacaucacaugagaaccaguuuuuggacaguaacaauguaaguuauuaaugccaucaagaaaaggaauaaaggcauagaaaguuaaccucugaguauccucuaaguaucugcagccuaguuauaucauuugauuucacaagaaugcuauaggagaagcuuuuaaggucuuuauuuuaauaguuaaagaaauggaggccgggcgugguggcuuaugccuguaaucccagcacuuugggaggccggggugggugggucacgaggucaggagauugagaccauccuggccaacauggugaaaccccgucucuauuaaaaauacaaaaauuagcugggugugguggcaggcaccuguagucccagcuacucaggaggcuaaggcaggagaaucacuugaaccugggagguggagguugcagugagccgagauugugccacuguacuccagccugggcgacagagugagggaggaagggaaggaaggagggaaaggugagaaggagggagggagggagagagggagggaaggagggagggagagagggaaaaggcucacaaaaaugcuaaccuucugaaauucauaccugcaaagggcaggcauuucuauuuuggggggccaccaccagucagaaaacuggcucaaugaacaacggguccagaucuaucaaagcauugcuuauauucuaaugaauuauucauucucauuuuguauaugauuaaacuaaaauuagaaaacugauguguuccaacacagaaaggcucuucaugacuuuaugacugaauuugugaaaaacauuucauuacauuuguaagauaguaauauuuuauacauacauuuguaucuaaugaacuaaauuuaccuaagcugcuauccauaauuuaaacaggaacgcauaaacacaugaugaauccgcuaucaccuucuaaaucaaaagacuuuuucauuugcaucaggaagcuuuugaaacuauuuaucuaagacuaagaguacauucugagaugauuuuguuguuguuauugucuuuaaacuaugguauaccauuguguagggcagaagaauuugaguggcugggaauuuuguuuuuauuuuuaaauuuuccaaaauaguacaaaauaaguuaacuuugccacaacguaauaacugaauaaaugccuguuuauagcaccuggagcaagauaaauaagaacucaguaaauauuuucaaaauugagcuaagaauuaugauguaauucaacugugacuucacaacaggaaaacauucuuuuuauaauuuuguuuuauaauguuauauaauguauaauguuuuauauuguaguuuugcucauauaaaauucauuuauauuuucuagaaaaagcauaauguuuuaaacacuaaccaugugacucauuauuauaauugcuaauauuggccuacuuugauuauucucacaaauuucauuagacuuggagaaagcacuccaucgcccucuugugguaauuuugcuguacuauauucgccaagaguucaaaaaaaugaacuuuuucuuuccacuuaaaaggcagagcaguacuuuaaccauaaacuucugaacuuccaaaggccguauauaaacuuucuguccuccacuguccauauacuuugugccuucucuucgguuucuaccucagaaauuuagucuuuucguugcuuuuuacaaaacauucuccuguuagcuuccaauuaccauaccaauagcauuuuucugaaaauuacuuucuguauucaaaugcacaaaguaauuacagaaaaucuucaucaucauacauaggcuuuaaauaaauagaaauaacucaaaaauguauggggcugagcuuagcauuuuuuccccaaaagaauagaucaugguaagucaguuaauaguccagaaauuucagagacuaauaauauuagacuaaguguuuuuuaguaguccagaaauuucagagacuaauaacauuagacuaaauguuuuaaguuuuuagaaguaaugaaagucuuuccucuaaaauagucuaacucauaugccauggccuguccucuuuccaacacaauagaaauacuucuagcuuuuuuuuuuuuuuuuuuuuuuuuuuuugagacagagucucacucugccacccaggcuggaguguaguggcaccaucuuggcucacugcaaccuucgccuccuggguucaagugauucuccugccucagccuccugaguagcuaggacuauaggugcccaccaccacgccuggcuaacuuuucuuuuauuuuuaguagagacaggguuuuacccaucaggugaucuugaucuccaggauggucucgaucuccugaccucgugauccgcccgccucagccucccaaagugcugggauuauaggcgugagucacugugcccagccacuucuagcuucuuagcaaaauuagugacauguuaacacaugccacuauaaaauagacuguauaaauaacguuuucuaaaaauaauuuauaaauaauuuauuuaguaucuaauauauuuaaacacugugaaaaauacaaaaacaaagauggauuccuacuuaauuaacuaaaaaauucacucuuacaaaucucaaaaauauucuuaaaagaauaaugagugaccauuuuuuuuuuggagacaagguaucucacuccuguugcccaggcuggaguguaguggugcaaucacagcucacuguagccucaacuuccugagcucagaugaugcucccaccucagccucccaaguaucugggacuacaggcucaugugccaccaugucuggcuaauauuuuauauuuuuaguagagaugggguuuugccaugcugcccaggcugaucucaaacuccugggcucaagugauccucaggagccaccgcacucagcuaugaucaauucuuaaagcuaucacauugugaaaaguggccccuuggguacucaaucagcauucaucucaaaacugaugaagaacgacuagaaauuagagggcuaggugaagaaguacauucuguacagagugguaagaacuuucaaaagcaugguaguaugccaugucuuggaaacugaagaugguuuagugcaaccaagcacagauuauaaauugggugaagcaggagaugaggcugagaaguuaguaggggccaaaguauaaaagguccuaaaugauuuguuaaggaagggugggauagucaagcauccacagcagaggaguaaagcagcaaaucagaguuugagaaagaucacuugcugcuggccaagcaugagagcucacaccuguaaucccaaugcuuugggaagacaaggugggaggaucgcuuaagcucaggaguucgagggcagccugggcaauauagugauauuccaccucuaccagaaaaaaaauaauaauaauaauucccagcuacuagggaaguugaaguggcaugaucacuugaaccuuggagguugaggcuacagugagcuaugauugugcuacugcacuucagccugggagacugggcaacagagugagauccugucucucuuuuuuuuuuugagauggagucucgcucugucgcccaggcuggagugcaguggcgugaucuuggcucacugcaagcuccaccuaccggguucaugccauucuccugccucagucucccaaguagcugggacuacaggcgcccgccaccaugcccggauaauuuuuuguguuuuuaguagagacaggguuucacuguguuacccaggauggucuagaucuccugaccucgugauccgccggccucggccucccaaagugcugggauuacaggcgugagccaccguggaaaaaaaaaaaagaaaaaaaagaaagaucacuugcuguaguauggagguaugggggacagacugaaggaggggaaaggagacagggaaauuagaagucauuuguaaugaagaggucaugaaucaaagcaauggcucugaugacacagagaagaaauuugagaaauaauaaaaaguaaaaccauuagauguuaguuacugugagaauguggggcguggggucuaaaagucuacauucuagcuuuggugacugaguggaugugcaauucccagagauaggaaaugguuauuuucagacauacuggauuugaggugccaccaggagauggcaaauagaucacucaguuggagagaaagaucuguaaauuaucgauaacuaaguaaugaaaggaaccuggaugagagaucacccagggagagcauaaagagugagaagagaugaaggcugagucugagauuagcaacauuuaaguacagucccauuuuccuguuaaaagggguacuuuuuuuuacauaaucauaaacuuuguucauguugggagaaauaggaaaugauuuagucuuucuucauuuugcagaucagaaaacugagggugucaaagagguuaugugacuuuauuuaaagcuguuaaucuguagaagucaaugagaacucagguuuccaaacugaggccaggcaguuccauccugcugcucaaaugcucuuucuaguacuguguccuucuuccccuaugcuacauaaacuguaagaacaagaauuggaagaugcuuucuucaguaauuguuugauggcaauaaauguuucugugguuguuauuuguacauaucaucaaaacuaaaauguuauauuuccaaggauaaccuuccaacaauauaguuguuauuaaauaagacccaaauuuacaauuuaacacaugcuuccuauucugggaaacaaggaaaugcccaauccuacaauuaugaauagcaaaugcgacuguaucaaauacacacaaagaauguuuaugagaugucucagagagaaaaauagcuauaguuuaauccugauaguccuuucuaaaucuuagguuauugagaauggguuuuaccagugaauucuaccauauaucccagaacacauuuucauaaugaaacucauuacgaaaagcaagacgcaagagauuucucugggcuaccuccuugguuuggccguugccagcuuggugacuugaugacgccaguggauccuacaggcuugaaggcagcugcagcugugaggcugguaaccccuggucugccagagguugggcuguccaggcucucgggcaugcuccguguggaagcuaccgaggaagccaacugcggagaaggcuccugagaguuacuguaaaggaaauaaagaaacaucuuuggugaagugguagcaacguaacucagcaauaauccuuucuuuucaaaauccauuaaucaaucaaaauccauugauuaaucaauucuuuagaaaagaaaaucuuugaagucaugacugagggauugccucucuuuaaaacugaaaaaaagaaaaaggagucagauugcauuuguuucauacuuugaaaauucuuucuaaaaggucuaccaugagaaauuaggcuacugaaaauuuacacaguuaaucauuuuaaaugauaaaagucucccugacaccucagcccuuaccaguuacucuuuuucuucuucuacuuuuuuuuuuuuuuuuuuuuuguagacacaggggucucacuaugcuccucaggcuggucuugaacuccuggccucaagcaaucaucccacuucaguuuccgaaagcacugggauuacaggugugagccaucauucccagcccaguuacuccuucuacucacauacucacucauucauaaauauuuaagcaccuacuauaugccaaacguugguugugcuaugcacuggccacaaaacaauaaacaagaaacaacccuugccuaacagaacacccaguguuaugcauucauaauaaagggaaaaaagagacaagacugccauuaucuucucucaaccaagaguuaccuguuuaaacagcaggucuuaucuaugcuaguuccaguuugguuuugaagaucauauggccuuuauuaccaagucauuugugauuuauaacuacauauaacaucauuuccaaaaaaaaaauaauaauucuagguagcccaaauuauaaaauauaugcuuugguaugauauaauacuauagcagugguuauuucuuucaaguaaaguauaauaacaauucuuaaaaguaauacuuucaggcugggugcaguggcacaugccuguaaucccagcacuuugggaggccaaggcuggcagaucaccugaggucaggaguucgagacucauuuccuccagacuuggucccaugcaccuuuuuccuuugcuaaucuugcuguguauucuuuugcuauaauaaaucuuagucaggagugcaacuauauacuaauccuguaaguccuucuaucgaaacaauaaaccuaggaguggucuuggggcaucucugacacuaaacaguuuugaaaaacaguaagaaugauuuucuugaaauauugcucccuauucuuguucucucaaaugcaagugucauagagccaucagcuuuuauuauuuuugaucaguuuaacuuaaaaaacuaaaaccuguuugaugauaaaacacgcuauacaauaaaaaacuuuaagcacuuuaauaaucuuuaucauaaauaaaaagucaaagacugccucccucaugggaauauugaacacaauuaucaguaaaggcaacacaacaaaaagcauuaauggaaucaggguuugaaauuacaauauagugguuacgagcaucuuuuaaaagcacugugguggacaaauuauuuuacaucucuaagccucaguuaccuguacuuuaaguguaccgaguaacauuuugcucauaagauuucuacgaggcuuaaauaagauaauuauguauaaagugcuuagcauacucuaaggcacagaauauaaccuuaacauauuaaaaggauaauuacauaauuaccauauucaaauaauaaaaguaagccuuauuuucaacauuuuuacucauuacaaauaaaugauuuuugaaauacuuauauagaggucauuuucauucugggauuaaauuacccuugcggauuugauuuuuaaaacuaauuaauaauguauauaaucacgguucuaagaaaaaaaauuaugaccaagcaaugcuaggaauuauagaugaaacaaaaauauauagucuggaaauaacaaaacccaauccaucaccauuuaauguguagucaucugaaagaaucaaaauuuaauuccaggagagcugccagacaaacugacuuaaaagagaggaauucagugucaagcuuaauauaguuugggugaaggaacugguaauaguagcccagguuaugaaaaaaccuagagaagcaugacagccauccucaaaugucugaccugcagauucacucuauguaacuacauuguuugccuaauuucaaggagugaaaucuacagugagauggauuugcuucaauaaaaggugauuccuaauauaaaaaaauccuccaaaggaaaaaaaucuguuccuaaugaggcuuucugugagagcccugucaccagaagccacuuaagcagaggauggaaugccgcacaacgaauuuagccauuuagccuguggaaggcucaguuauauuucagcaugacauacaaagccuucuaagauaaagcuacaaucuaccagucuaaucuuaucuccuaucacuaugcuauacaaacuauauucuggccaccccuacugucguccacaauucuaccuuuuuuuuccagauucuauauacgagguaccucuuccuacucucucucugcacccauccacaucuuauccauaauuaauaauaauagaaguaaauauaauaacgcaucaugcacuguguucaaauuaacauacauuauuuuguuuaauuauugaagccuccagcaguagagacacucuuuuuaauucaggacauucguuaacugcauggcauauaauuuuaugcugauuuguaauagcuguuuuaaaauuacuauuaauagucacuucuuuaugcaucuugucuccuccacuucauuauaaacuaccugagguuggggacuuuacugugacuguggguaaauuauuuaaacuugguaaauuuccucaccuguaaaaugguauaaaaauacuaccuuuaucccaagauuacuguuaagaauuaaauaagauaugagauaauacaugcaaauuacuuagcacagugcccagcagagaguauaugcucaggaaauaaaaaggauucuuacuucuacauccuguaauaucuggucuggugauaauaaaauuagcaauaaguacauuuacuaaauaagcauccuucaucugcacuaagaauuuugcuucuaaauguacuauggaucacuugggugcaggguauugaccccagacaaaaucuugcuuccucaauucaguuuuaaaaacacacaauacuucuuaaaguucccacagaauaaguaucauauaagcuaaagaaauuucaauuuuugauuguaccuuuuguacacagguuuguauacguucucucucagaggccuaagauguuuaaaucuuaaacaguacucuuuagcuacaaagggcuuggaguagacuauaaagaauucauagaaaucuaaguuauuaccaaggcaguuuuuccauuuaugcuuaaauccagaauugaucugaaguucaauuuucucugaggauaaaauuuaauugaaccauuuaauguuguaauggaagaauauuaggguuuaucuagacaaucauauaucugaauauaugaugcccaauccccuaaaaaucuaacauuuuuucccuaauuuuaacuauuagcuacuauuuaaagagcauacauaaacauuugguauuggcugggcacaguaguucaugccuguaaucccagcacuuuaggagaccaagacagaaggacugcuugagcccaggaguucaagaccagccugggcaacacggcaagaccuugucucuguaaaaaaacaaacauuguacuaaagaugaaaggauuggcugggcgcgguggcucaugccuguaaucccagcaccuugggaggccgaggugggcggaucacgaggucaagagauggagaccauccuggacaauguggugaaaccccgucucuacuaaaaauacaaaaauuagcugggugugguggugcaugccugaagucccagcuacucgggaggcugaggcaggagaaucacuugaacccgcgaggcggagguugcagugaacugagaucgcaccacugcacuccagccuggugaaagagcaagacuccgucucaaaaaaaaaaaaaaaaaaaaaggauuaaaagucacuggccauauuauuuuauauguaggaauaaaauauuaucaauaaucacuagcaagaaacuucuaaugauuaauguauuaauauaguauagcauuguuacuuaaauuagauugcuauuuuauuucugaacuaaugauucaaauauauuuucuaaauuuaaauuauuuaagguguaacuaaauccuaaauaaaauuuaaauguuuucuaaauaauucuaaugaggaaauugguguaaauccauaaauacccuuggcugauaccacagauacugauauuaaaauaugguuauuuucacuaaucuaccaaauaaacagggacugaaaugaaauaauagauugauaugacuucuaucuuaagaauaccaagguuuuuguuucauuuuuucaaaaacugaaaauagagacaaguacacuaauagagcaaaaguuuauuuucaagauggagcuaaugaggaaaaacgaaaaggaagaaaguuaaccucagugucagcaaccugauuuaucuugaguuuacauuccuuucccacauuuguuuaacuguugcaacaaauaaagcucagauacagauuaagaauacuugggaauuuuaaguccauuuucaaaggaaaaacucugcuugaagaauacuuuucagaguaacuuauucuaauuacauguuuaaauuuuuuaaaagacaacuguacaucacagguuuaauuaucaauguuuaaguuaaacaguuuuuggcuaaagucuuaucaaaaaaugguauacuaauauacagauucugaaauggcaggaaaggaauauuucuggaaaggccuuaguuacaagcaauuguuuauuuuauagaauuccucugucagaacgccuuuaaaagaccuggacauguagagaggguaaugagugucuuuccuuccagaauucuauauuuagaagaaaauuuuuaauauuuuccuaagcaaaauucuuuaacuuagaguuuguaaaccaaaauuuaaaguauaauauacuaauauauauuauaauggaaauauauuuuaauggaaaaauauauuuuaauggaaaaaaauaaauaaaaacuauuauacuauauuuauacuuuuaaagauuacuaucacacaaaaguaaaaaucaagacaaaaugucucugacacauuuuaugauuacugucauacaagauaaaauauaucuggucuuuuggguacuccucucacuaaguuagcaagggucuccucaggaaaccccagaaugccaacuagacuaucuuuuuagaaaccauuucccucucucaauaucacacuauauuuugucuguguccuauuguuaauaagaagcaauggugaugacuacuaauacucgcucuagagguggggccauagauagcacagucaguccauaaaugcuugcacacuuuugcauguauaugcacgcaggcacuuuucuggagagaggaucuauagcucuuaccggguccuaaaaguguggucuaaguguacucaaguacucaaaguguacucuaagcauggucaguaaccugccucuguacaaucucagcccugacaaugaaagacuucugcucaaugaacacucacaauauucuaauauaauuucagguauugucuucucuuagagaaaaauguacuaugaucuuucucccuuguuuaaacaccucccuuaaaaguuacccagaauaauagguaaauauauauauauuuuuuguuuguuuguuugcuuuuguuuuuguuuuuugagacagagucuugcccugucgcccaggcuggagugcaauggcgcaauuucggcucacugcaacuucugccucccggguucaagugauucuccugccucagccuccugaguagcugggauuacaggcacccacugccacacccggcuaauuuuuuuguaucuuuaguagagaugggguuucaccauguuggcuaggcuggucucgaacuccugaccucaugauccaccagccuuggccucccaaaaugcugggauuacaggugugagccacugcaccuggcccagguaaauauauugaaaaaucucuauaagaaugguuagaauacaaacaucuaaucaauagacagccuaguuauaucagguuuucacuuugccugauauauguuguacacuuugccacugauuuuacguucugucaaaaaucauuguauacaguuauuucauugagauuauaucuuuaguaaacacuugugauaguuuuauuuuuauauuaugccuaaaaauuucuggcuuaauccuuaagcuuaggagaaaaaucuauuuugguguuuagcauuucccaaauacagugaaaauuaaguugcuuuuccaagcaaaaccuucauauuuaacaacuauaauauguagccaaucaaaauugaucauuaucauuuucauuuuucuaaaagcuaugucuuaccaaacauuugaggauucuaauauagcuauauccaugcaaugaaagacagaaauucuacugcaauuuaaauacuugcacaauuacucagaauugcacaccaaacaucaaguaugcuaaggaaagaugucacaaaagguuaaaaggugaguuaauaaucauaggacauacugugcuugugcauuagcaugccacugacauuacaguucauucaaaauugggcacaaaucccuagaacucuagcauguucucaccuggccuguuaauacagugguggaguguuagagaaagcaguaugcuuaacagcgaaccaccacuugccauaucugcucccacaguaccuuguuuucuuaacauggauagagccaguugauuuccuugaggacugcagauaggaaaaagcuaaaggcuuggagacagggcugaugcaaagugcauccaggagugagugauaucuuucaggaguguagagccugguaagcauuuagaaagaguuuuaagucaaaaaucacacuaccaauagcagacaguaguucuuaaggucugugauuugaaacauuuccuuuacgagagagaggguggggagagagaggaagaaggaggggaagcacaaagcagcaaaggaggaugaaagaagauaaaaagaagcaccaagaguuuauucuaucccuuuacucauuaauauaacauugauuagauuuucaccaaaauggucaaagccaucauuuagaaagcauuugaagcucuaaagacuauagaaauuaucuaaaauccccaaaccacgaaaacaaaaccaauuuauucuuuuuggggaaggacacaauuuuauuuuuuuaggggagaguagucuucagaucacccuuuaaaggucgugggagaagauaaaaucaauagaaacuuaaaauauucauuaauacuuuauuguuucaaauauaaaaccugaaaaauguaaaaacccaagugacaauaagcacacaacauguaauaauucacgcaacugagaaauaccauacauaaauggcagcaguggagagauuaaaaggugauuuuuuaagaaaaaaccaguaacaaauacugugagauuuauccacaaccuagauaaaauaugcacuacacaauuaagagcucacaauagagcuaugauugcuucaaaggggcauguuuucuuugaggugacuuguaagacagugaagagcgcugagaucacaucagacagacgcuguacguguaguucuaccugguuuuuguagcaauaacagcagacacaguagcagcugcgcugcucagaacugcagcacuauaucuagcaggagaagagaagguagaaaaguuucgaaaaccuucgaaagaaucuuccacauuuccuguggagcugcuuaaacugacagguagggaaagaaaaacaggagaggaacaaaaaggaaagugagaaaggguagcauaauaaauacaagagagcaaccauuucauauuaauaucuaagcauuauaaaagugugaaguguaagaaguguaauaggaguucaggccauuuagucugugcuccuacgguuucucucagaagcuaaaagcugauguauaauccuccauaaagcugauuuggacauacgaauacucuucaauguauuuaaauucacugacuguauuccuauuuaaaauuaccucguaucacacauuugaagacugacuugccaacaacuuaguagaaaauaaaucuaaacgugaagucugaauuucuauaagcauagaaagaaggaaaauuguuuuuucccaaaaagucucuauacaaugagcuuuaccuuagagacgauauauacugacagcgugacauuuuaauacgacuuauguuuucaggagaagcaggcacaacagacugcuuaucaagaaggagcaauauauauuaacaugaaaagagauaauuguuuaaaauauugagaguaaagcuauguaaggagggaaaguaccuuugcgaaaauaaaccaauguuacagugaauaugaucaaaacaucaccuuuucugcugaguuccauauuucuauuuucaguuaguucuguaagcauuuuuaaaugcagguauagcauuuuaucuucagaacuaauaaguauuuauuaauuaaaauaauaauuuucccuuggccuuuuagaaaagaucaaaugccuggacaggaaugaacauucuauaauuaugacuguagugauuuucaaauugguuauuaaguaaaauaaaaagaguguauaaacguauagugugaaacuuguagucucucuucuucauuuaugggugcaacuuaucaaugagcaucaucacuacaguaugaccacuuuuuucaauggccaccuccaacacaaaguaauuacguggcaaacucgaccucugguuaaugugagcuuagaugucugaaacugugaggguugacauaagucucuuuuuccugucaaaagaauuugauaagucauucuuccaauuuccacaggcuauaacuuuacaaaaacgauuagaaaaaaaauuaaggaaauaguugcaagcuauguuuucaaaaugauacagaauuaaguaauuuuucuuuuggucuaucuaagucuauccauuaaauuaaacuguauggcuaaguuuuaacaacuguuaaugaaugguuagaaaagaaaacgucagggucuggagagagaaaagaaagauguuaucaauggcagagcaggguagguaacaugaaugucaaagggaauaauaaaguuaucaugugauuccacuucugaguuucugcauuacucugcaucucugagacaaugguuacuauuuggagacauaauggugaauaauaagaacuagaaacuuaguauauugaaaaaucacuuaaaaauaaaucagcuaauuuuuucuucuccaggaagaaaaggaaacgguaaugaauuucuuucaucauauugcaucacaaauaauagaaaaggauuuaguaucagcagaaucuaguuauaucucauaagcaguuacuuaucugaguaucuuuagaauaugucugggcaccugguaacauauuguguaugaccuuuaccugucuuugaggcaaaugcucuaauucagaaaaggagacuuaugacaaagccagcaaguuaacuuuccaaaucacaaagugaauaaaugaaagagauuucuugauucuauauuagaaguuauaaacuaaacaaaaauaaaaauauaaaagcagugagaagcgcuguuuugaggcugagugccaagcuuucuugucuauagguuaaugauguccguucuguaaucaacaaucauuuuuucuuuuucaaacaguuuaguauauuuaaauucacuaccagcuuugaaauuuagaaacaccaagaaggaagagucuaccuagaauaaguaguugcuacuuuagaaaguguggccacuuggcuaccaauaguuaacaccugaggaguugcaggagggugagguccacucuuagguaggucuuccagagcacugucaaaccuaaguuuucagaaguaaaauauauaaggcaaaggaaauauuaaugaaaaguacaaaagacauuuaucaucuauaccaucuuuuaaaugucuacuaguucaauuuugggaacacuauugguuuucuuuuacuucacaucuacaaaugguuaaaacaaaacccaacacuucugccaccacaaaauaugggcuguuuucccccaaaauucuuuaauauggcuacuucuauguaauaaaauauuacgcuuacaggggaaaaggagguggaaauucccauucacugacuacguguuuugaaugcuuuaaaaaaacuuaggcuccaagaaguuaauuaaguugcccaauaccauacaucuaguaagggccagguggaauuuggaccaggucugccuguauuauuuccacuauauaauguuuuccagaacauugugagacagcuaccuaaaaguguccuacaagaaagcaaccaaaaguuucacgucagaauguucugucuguagaucugaaguuauauggauguuguguaucuugauguaaacaaacuauuuuaucugcucauacaacagaugguggcaugcaaucguaggguuuugaguauucuuuacuuuaauuuucugauaucuguauuauuuuuuauauagaaugccagaaguaaguucuagaagaacuauugccagguaaagaaaaaaugaagaacucaauguuaaguucaaaaaaaagaaagcaccaaguauaaaacuuugggaagaaaaauaaaacaggaaaaauacaaaauuagccauuaaaaaaaaaaagucuguggauaaaaaaauagaauggucagucauguuacacugauuuucaaauggauuaaaaaauaggaaaggagagguuauauuccuguuuuuuauuucacauuagucauaccuuuaccagaguguugaaaaaacguagacaguugaagaaaaaugucaugauagaacuaaaacauuaagagguagagcagaaagguuaagagacuaagaaauauaagaaccauaacaugggucagcaaaccacggcaugcuggccaaauucaggcccugccugucuuuguauggccuaagaauaguuuucauauuuuuaaaugguuaaugguuaagaaaaucgaaauaacaauacaaugacacacaaaaauuauauaaaauucaaauuucagcauccauaaauaagguuuuauuggaacuuuauuggaacauagccagguucauucuucauaugucuauggcuacuuucauaugauauaagaacugacuaacugugacagaccauauggccugaaaaaucuagcacauuuccuaucuggaccuuuacagaaaaaaauuugcugaccccuguccuagaaaauaacuucucuacacagcuaccagagugaacucugaaaaauacaaaugccaucaugucuguugcuuagaccuuccagcccugcucuaggugaucuugggcuaccuguucuucagcauuaugucaugaagcuuguccacucucuuuggcuugcuccaguccacugggccuuccaucucuuuuugaaauaggcccugcuucuaucccccuccuggccucugcacagucuccuacuccucacuguccucagcccucguucuaccacacccucacugggauaacuccaaggcauccuucuggccucaguuuaaaugucaccuccucccagaagccucccugauuccccaagccaccuuagcugccccugucaugugcugucauagcagucuguacuuuucacucauauaauuuguuauuauuauuauacaauuugugucguuauuuguuuaaauccagacagucucagaagacuagaagcucccugauaccaauaguuacugauauacaugaaguguaacaaagugcuuaccauaaaguuaacaaauauuugugaaauaaaagcuugaaauuuauuuuuuccaaauaacgaauucacaauauuuguaaauugaaaaguauuuguaccuucuagagacugugucacaauucugacuaaacagauuuuugccuaaguggacacaugauaacuugaaagaaaugaauagugcucccaaaauaccuuuaaauaaauauuuuaauauuaaaaauuuauaaauauuucaggaaaaaagaaaauaugguuauaaauuauuaucaauaucuacauauuaccacaggacagauuaaaaaaaaaaagaaaagggagugaguuggagaaguugagucuuccaacaucuccgugaaauaugcugauuggaaaaaagcugagugccauuugagaauaaauuucauauaaaucaugaaauaccuuugaagaugaucuccaagucaucuuccaagcaaacgcacuugggaaguuucccauguguuacacguauugauuaucucuucauugcuuagccaaacucuaaaaaccugacuuaagacaaaaucuuaaacuaccucauuuacuauggaacaaacaucaguaucuguucucauucugacauuccccaguguuuacuccuuuuagguuggaggaagcucugauuaauagcauaucugcagcagccaaauuuucugccuugucuugcuuaaaugaguuuucauuucucaccagagaggguuaugaagacuuccucuuucccagauaaagucuuucccuuuuggcuugcuuuucucaggauacaugaacaaagagcuauucaugcauugugucuauuucugcaccaucucugcacuuguucauuacggacccuugugaacaaucuguccagcaaaucaggugugaguuguccaucuacuaauuuaccauccuuaccauccuguaggaccaguaagaagaauucuuuuucuauccccuaguccggacaacugcccaagagcaacauucuaaugcuguguacuccaucuccucccuuaaucucaguggccucgauuaauccaucucuccuucucuacuggaugcuuuguguccucuuuuaaaaugcaguaguuucucuuaaaaacaacagacuaucuuuacuuucuauuuuccuguuccacuuaccagccagacucugaaauaacauuauacucacuugacugucagucuucagccaucucaacuagaccccaacccccaacacucuaugaaacugcaucuugcuaaggucacugaugaccuucuuguggcuaaauucaauggacaauuugcauuccucauuuuaaaaacugugguaaaaaauacauaacaugaaacuuacuaucuuaacgauuuucaagugcacagucaguagugugaaguacguucacauuauuguggaagagaucuccagaaguuuuucaucuugcauauucgaaacucuauacccauugaacaacuaccccuucccccucccccuagccugugguaaccagcaccauucuacuguuucuaugaagaugacuacuuuaauucuuuuuuuggagacggagccucacucugucacccaggcuggaguacaguggcacaaucccggcucacugcaaccuccaccucccugguucaaacgauucuccugccucagccuccugaauagcugggacuacagguacgugccaccacgccuggcuaauuuuuguguuuuuaguagagacgggguuucuccauguuggucaggcuggucucgaacucccgaccuccagugauccgccugccuuggccucccaaagugcugggauuacaggcgugagccaccacacccagcccgaagaugacuacauuagauaccuccuaagccugacccgaagaugacuacauuagauaccuccuaucaguggaaucauacaauauuugucuuuuugugcacauuccucauuuuacuuagugucuggcucaauugacuacugcuucucuuuuccugucauuugugccucaacccuauuuaccugccuguuuuuaguaguauuucacuggcuauucuguugcccgagaguucucuguccuaggcuuuauuucucuguacucugcuaucauuaguacagugacauuacucuguccuguagcuuuaacaccaccuaauguccagauuuauaucuagcccagcugucuccuaugagcaguagcccauauuuugaauugcccuuuaguuauuaauaccucuaauaucucucugucucaaaucaucuuucaccauguccaaaugacucuacuuccaaauuuauauuuuuaaucuaucaguggugugauggguccccuaccagguuacuuaaggguguuauauucaaugcuugaaacuugagggccaggcaguuagcuaaggccauggugccuagccgagaagcaggggucccugagaacgcaaacauccugaggcauggcugggaauauaccaaggaaaaaagucccagcacacucacacacgcgcgcgugcgcacacacauacacggcaaagagccagaaaauuagcuuaaaagcagccuagagaugggaggcgguguggaucucuuggagccauacugcugcuguccagcagugcccuguauguauguccuaauaaacucaucuacucaucaagcuggacuugucugagucauucucuuggcaacgucccaguuuucccaguuugcggaggguugggggggcauuacagucccaaguuuuucucauuacgccauccaucuaccccauuucucugcuaccacccucucucauccagacuaugauaagcacuaacuuauacuagugauuuaagaacugucuaacugcucucuugcuuccucucuugccaaauccugccccuuccagccccuguguuuucuggcaugguuuaguauagcccaugugccuuaagaaaaaacaacaaaacacauuuuaucaaaucauucuuuugauuaaaaccuugcaacaaauuccuacugcaguuaggauaauacccaaagugcuuaacauucucuacaaaauccccaaaugaccuguuaugaucaaccucucaauuaucuuuccacaccaauuuucccuuuauucacugaacuccagucacauuagacuaccuguacaaugucuugaacaaacuaaguucuuccaggauucaggcucuuggucauuaaugauaauauuauuacuuuuacuucauuuuuuaacuuacauguaaaucauuuguuaauaauaaaagcaaauauuuuuucaucgcuuaauauguaucaucauauuuaauccuuccaauaaucuuucaacccugguacuaaaauuauccuugguaauuuacuaaaguuaguacuuugguauaugguggagcuacuguacuggcacccaugguuagaauaacuucaguggaaugaggagaacaaaaaccaggcugcaggggccuggugugguggcucacaccugcaaucccagcacuuugggaggccaaggcagguggaucaccugaggucaggaguucaagaccagccuggcccacauggcaaaacuccgucucuacuaaaaaaaauauaaaaauuagcugggugugguggugggugccuguaaucccagcuacucgggaggcugaggcaggagaaucacuugaaccugggaggcggagguugcagugagccaagauccugccauugcauuccagccuggaugagagagcgaaacuccgucucaaaaagaaaccaggcugcaggaugguaaaguucucuaucaguaauaaaauuucaaaauguuaauuugggucauccagauuuugauggaauuauaugcaaacuauguucuaagaagaaaucugucacaaaggcgaaccuuaaaaaaaucuuaaaaauaaugcaaacauuuucuuuuacaacauaaaaauggcaucuucaauuaaaauuuuaaaugcuuccuauuucugaauauaaauaugauaaaaugaucaaauagacuagugacuuauuacauacccacacgacgaguuuucaauaagaggaaaaauucacaaaccuugaaauauacguuuuauuaaaagguaaaggaggaaaaugacaauaaauuguccuugguauucuacuuccacaaagcaaacuuuuauguaauaauauugcuaagcagaaacuagaauauaucagagucauagcuagauucugcccuuuugggguucacuggcauagcaaagcuauuuauuuuagccaaggaaacugcuaggcaugugaaaaaaauaaguauuuucugcuggcuuccugacaauaaauauguugaguuugcucauuuguaacuaugaucucccuucuggaaaacuaaaucaacuaaaccucaggugggaauuucugguacucucaaucauaagcuauccauuuauuaauugguuuucaauagucuuuguuucuguagcagccugaaaauguuacuugagccugcuaauucuaccuuuauuacauagauggcaucuugagaauuuaaaaauaagcaaguagaguuauccuugcuuugcacauggcagacaaggaggacuggaucuuagaggcuaaaagguuaugaaauaaauuaucaaacaugaaaauacuacccaaaaugagguugauuuuuuguaagagcaaagaaggacuaacucaaagaaaucauuuuucuaggagcuucccacuuacuuaagcaaggaaaagauaggcaaacaaaaagggauauugcugaaggaauuggaacuuauuauuuguuguuauacagugaacguuuguguccccccagccccaaaucauaugucaaaauccuaacgcccuaugugauaguauuaggaggugaggccucugggagaugauuaggucacacaggcagagagcgcucaugaaaggggucccuuauaagagauaccucagagagcucccucucuccuaccauaugaggauacagugagaaggcaucaucuaugaacuaagaagcagucccucaccagacacugaguguacagguaccucgaucuugacuucccagccuccagaacuaugagaaauaacuguuuguuguuuaagccacccaguccaugauauuuuuguucuagcagcuugaucugacuaagacauuuguauuagggaaagaauuuccucuuugaugauauuuuccauuuucucacaucuuucaaacuauggcuuuauaacucuuacacauuucucugcuguugccacuccauaauggauuuuaucauuauuaaguguucaauacuuaauauuuuguaaauucuuuaguuaucuagaguauucuuugaucucuauauagaaaauguucuauagggucuaaagccagguaaagaaugagcauuuuuuaucuauacaaaaugcaaauauaauaugaagauuuuaacguguaaugacaacuuaaaagucacuuggagcaauccccugauaaaagcaugacucccucaguaacugcagggcacuuaccacuaaccuuuggcccucucacuuuccaucaucaguuucuucacuggaucauucugauccaugacuucagagauuuauacuaguuagaaauagcuucuuauaugugacaaaaaucugccauuuuguaguuuuaacauaauccuaauauuagaaaaagaauuuauaaacaauaaagguauuuuucagauucgggauaaagaggagaaaggcauguauuauuuaauaaauaguguuaagucaaguuaccuguuugaaaauauaaaagaauacgucugaaugugucccacugaacagccacccacagugaauccuuaaagaggccacaugucuugagaaagccagcacaguaccucaugcuccagugagaguguccuguuggcauuacagaacaauagccaaguggcuacacacucacuagccaaauauucuggaucauaugucccaaacgcacccauucaaucacucauugcauucacucauucauucaauaacaaaaaacaauuacuugaauucccacuuugugccaagcaccuucacacauccuauugucuacuuuaauuuccugcccucaugaagcuuacauucuaguauccauuuuccuguaauggacucugaggauuaaucacaucaugcuucccacuuacccuuguuuaccugccacaaucagaaguucaaaucccuugggucauccagcaucuccuggacucuuauuucuucacugcuuuguguaucuccuaaucuaaucacuucaccaucuaucacagcccucacaaacccauuacuguuuuacuauaccuucuuuacagucucuucaaacagauguugcuuuuucuuucauaugccuuauucuuauaaaccuggagauggagcaaguguccuccucauuacuguuuuaacuacuaucagucaucuuugauccuccaaaaucccaaguccuuggaggcucacuauaccauucacuauagggccuucccacuguuugucucuaccucacccacuguuuuagcuccugacugccuuccucuucaccccauuccaacaaucucucaugauuauuucaauaaucaugcacaaucacugaauaaauaaccuuacuauugaauuguuugauuucuucacuuuaaugauacauuccccuaaggcuaagccuuacaccuagaauauaucagcaugcacuccaaaauucauaaccuuuaauuuuucaucaacaaguacaacaugucugaaaucuaaauuuuaaggacccuuuuuuuuccucacucagucugcuaucccucgauucuauuagcuuuuuuuacuguaccaaauugacucugaccaaucucauuagcucuuaucaacugaacuaauucuuuacaaauucacugacuuaccaauuuuccacucuccauuacccuccaccucaauauuucuauacaccuaucagcucuuugucuaaaauacaaaccuauccaaugcucuccaucuccacugccacuacauugcuuaucugaacuauggggauacucauaacagucuccugcucccauuauuucucccuuacaauuaauuuuccauaguauucagggugaucuguuaaaaauauacaucagauguuacuccuaugacuacaguccuauauugggccucucauuuaauuagaaaaauaaucugaacuccuuaacacaaccuacaaggcagucaauaaucuggccccugaguacuucacguaaucucuuaucauucugucucuuauuuacuaugccccauucauaauggcucuuuuucaguucccuaaacacaccaacugauuccugucucagggacucugcauuugcuguuccuucugccuggagaggacaucuccacauccucaugacugacucuuucagggcacucacauuucagcucaaauuauuuucuuuuucucuaacuagagucuaaugaccuuacucuuccccuccccucauuauauucauguuguacuauauuacuuuauagaauauaucauaaccuaaaacuguauuuuuccuuauuuuccuuuuucauugcaugucugcucuacuagacuaaguucauaauagcaucuucauuuuccuagaaaccagcacaaauaagugccucauaaacauuuguuggacguauuuauuauauauaauauaugcuaaacaugagcaggggccacugcuaauaagugaggccugagacuaaaggagaauguuuagaggaaaagacccacuuggauuuaggucaaagaaccagcccagauuaucgucuaauaucaacuacucauauaacugcuucucacccaaagacagcuuaauugucucagugagucuucaguugauuuucuugggauuaucuauguaauugucagagauuaacgucaaggacuagcauaauaugccaccaggugagauuuacuagugcagaacagaacuacugcuuaucauuuuaggagccauacgacaugacuaacucuuacuggguuuauuuucuuuuaaaauucccacuucuccuuugauauuuagguuugccaaccuuauugguuauugaacauguaugcuauaucucccugcuucaugguacucacaaauaugggcagcaaguuguggaucuccucauccaagucacuaauuaauccuuuaaacaauauucuuugguuaaacuacuucaaccuuuuaugcauuuaaaaacauacuuaucaaccucauuucauuauuaaauucaaaggaauuuuaagggucuuucuuugaucaauuguuaaaauuaacaucucucccaaucuauccaaaauguguuaaguuggaugugauguguuucuugagaacuuauucugggucuuggcaauucugcccuccucuucucagugcucacuagccaugcuuuucagaaugccugcuugacuuuuuuccagaugaucucaaguucaaauuaguuuaugguuagaaguuaacuuuuuuuuccuuuuuugaaaaucaguacuauauuuuccuuuuaccacucuucuauuuucuuucccauccaggauuugaaacaaacgcagauaguacucagugaaucucaucuguaaguccucuacauacccugagcgguaagucauuaagaaaaagagucuagugagaaccauuuagaaucacuuucuguauggggggcuucaaaucccuccaggauauaaauaaaccucuggguuuugguuauuuuuauuugcugucuuucaacacagagcauuuauuaguuucaccaucacccuugacgacaucacuucuguuuuuuacuuaucccuugaauagauguuaucugauuguuuuaccuuucauauauaaaggauuuuugguuuuacuguugguggguguacuuuuuguaaacauaccugugaugcugcuccaacaucccugcuaagagcuuugcugauuuuaacaaguaugugauuccacugcuguaagccaaggguaaacuugauuccaccaaauuugcuuaaguguuacuucucuguauuaaagcuucaaguucacucacugucuaaccaaugauacacagcuaccugaagacauuaaaaucuuucagaaauuuauucucaauuuuuuucuucuuauaaauuacuagcauaucaggauaaagaauaaagugugugaagaggcaaaauaaguguguaccacuaacuugaaaauuccgcaacaggugucaccuuaguuuuaacacauucuucuguucaaagauauaaauucagaaucuuugggaguaguuaauuaggugcaucugugagaugaacaccuauuacagaguuggacacaggaguagcauucaaacaauacccuuuaauagauacaaaaauagaaaaacacaaaagagaauacauggacacaugagacuaauuuuuagauaaaagcaauuuguagauaguucuuuaauuugacuaaaaggaaagugaagaugucuaaauauccauaccugucauaugcuuuauacuauauaauauaauauauugguucaauuuucuuuuauaaaaucauaucauucuaauaaacaaaccuucacauaauauaaaaguaggaaaacuuggacuucugaaaccugcaaugugucugaaauacauaaacuagaauagcagcaaaagauagaaauuaagccuuccaugauuuugaagucugccuaucaucaaugcucuuaauuaacuuuugaaguugugauuucuuuugaagaaaaaaaaaaacuaaagugcaggagucuucuucaauacugaguuuuaguaaacauaaguuucuuuaauuaucugguuuaauucaauaaaauaauuuuauuguguaauuuacuucucuaaaggugaaauggaaaacaacuuauccaaacagcauuauaagagaaggacuaccggucugacaaaauucugccucuaaggaacguggugaauauucugggauuaugaaagccaacuuggcauauuacgaacacugacuuuuauaaauaacagaucuuuauaagugcuaauaguacacguuuaauuugguaaacaacaacaacaacaggcauuuccagcauuaaacagaauuaaacaauuuggaugugaaaauacauaggaacuauguuucuuggcucaauaauuuuuaaguacuagucucacaaggaacugaaguguaguaauuacuuauguucaacagauggcgaccuuuacauuguagucagcaaaacaguauuugcauaaaacaguauacucaaccagcagaaaaucaaagaucaauuacaaauauuccauauuuugcaagagacaaaaauuacuugaucauauacaccauuuuuugacaggugguuuuuuuacagguuggcaaacauauuuugugggaauguaucuugcuguucuauuuuuuggggcaaguccaauugcuaauucuuaucucaugaaaauaugugcaaauaucaggcuagggaauaauuccugcuuucuuugcauagcuuauuaagaauagcagcuuuagguaauauacucaacuaacgugucuuaauaaggauaugucacaagacaccaguauguuugugaaacacagcaauaaugaagacaugggaugggucagcaaacuauuucuucauuaaucuauuuugugugggucaaugaauaucaacaaaguuuuaaaaaaacuuacaaaacaguauguucagaaauauguaauuuaugcuuccuaaauuuucaaugaauguacaaagguaauagguuucuccuuaagaaguaacauuaacucuauucuuccagugcuaaaauuccauucugauggccuucagccagaaaucaugugggcauuauuuggugcuagaagauagccuacaagaagacgguuucccggcucaacauaacuguuuuaguuuguguauuguaauugguaaccugaguuugaauuguuuuucuacuaaagggagaagcagucacuccucuccuaaugagcauuuuaauucuuugacaauugggaagccauaucauaccuuauaauucuuaaccauugauaugguuuggcucuguguccccacccaaaccacaugucaaauuguaauccccacguguugagggagggaccuggcaggagguaccuggaucauuggggugguuuucccaugcuguucuugugacagugagauuguucucaugagaucugguuguuuaaaaguguguggcaccucccccuucucucucucuccugcuccaccauugugaagauguaccagcuuccccuucaccuuccaccaugauuguaaguuuccugaggacucccacccaugauuccuguuaagccuguggaacuaugagucaaucaaaccgcuuccucauaaauuacugucucagguaguucuuuuuagcaguaugagaacagacuaauacaaccauaauaaauaugugaaaaaucagaacccaucauucuuuaagcaagggagauaaagaaaagacugauaaaaauguuuugaucauuauuuuagauauccuuuugcuaaacuaaaauaccacuuuauacuuauauauagcauuaaaaauuugaaacuacaaagauugauauuauuauaauauaguagaaaauugagaacauggugagaucaagaaaaacauuuuuuaacuuaaauauacuuugacaauaugauuuuuuggcauaaaugugugugugugugugcauguauaacauaauuaucucuaaauaaaauuuucuauggucucccauaauucucuauucugguaggccauauauuauuaauaacuuucuauuauuaauaguuacugauaagauggcuguugccucuuuuaaaucaauguuguagauuagauucuuauauuuaaacauuuccuuauuaaugauaaaauuuucucuauuaguauuucuguucagagcacuaacuaagcaucucauauaccuauuucuaaaauuguuauucucuccucaugggaaucaccaugcucuucauguugaaacacaccaaaaacacaaaggaagacauuaaaaaacaaauguucauuuaaugaaaaaaagucuuucacuaauuaacaaacagaaaauaucaagaaaaaagugucaacuuuuggcaauuagauuacuccaacaaaguacaauuccccuuucaagugacuuagguaugguuucacuuuacuuucaauauagguauuaauguuuugauauuuuaaugucuugauauuuauaucgaacuuauauuccauuagagauagugugugccacgcuguaaacaacuucccuaaaauuagagaauuggaaaaauaugcaauuauauauaauuaguaacuuuuaguaccaaacaugguggggcauacuaaaauucucacauucaaagaacuuuuacuguaguugagaaaagagaauauggagacagauaccugucauccaaagguaaaaaggaagcaccaaaagagaggugacaacaaugcuuccaauuggguccaagucaaugauucuacuuaagaauaauuuuuucuuaaaugacaaucugucaaaaugaggauuaguauacaaagacaguuaguguauguuauggaccgaacuguaucccuccauuauucauauguugaagcccuaacccacaaugugacuauuuggagauagguccuuuaaagagguaauuaagguuaaaugaagucauaagggugggacccugacccaauagaacugauguccucaugagaagaagagaccccagagcuuucaugagaagaacagaccccagagcuuucacucuuucucugugcccccacagagaaaaagccaugugaggagacacagugagacaguggccauacacaaccguuaggaaaggccucaacagacaccaacucugcuggcaccuugaccuagaacuuacagucuccagaacuaugagauaauaaguuucuacugucccaccuaaccagucuguguaucuuguggcagccuaaacugacugaaaacaguauauuucuaacugaagaaacaguucaccagagaugaauuuuauaaucuuaacaaccaccuucaagauuagucauuucauugaauugcuaggccuuuacuuacaguugcagcuacaguugcauccaggaagaaauacgaugacuacuagaaugccuuuuccuuccaauuuauacuauuacacuaagacuuuuguuuuuaaagagcucaaacuucgguaggaaacuuaagacauguagaccaauauccauaagacaaauuagaaaaucaagugucaaaugagagguaaaaauuguguagcauguaaguucagaggugacaaaauuauuccuacugugggguggggcaguggagagggcgcucuguaggugagaauguaugauugugggaaggaauaagaaaaucuaggccagacgcaauggcucacaccgacgauccuaggacuucgggaggcugaggcaggcagauugccaagaguucgagaccagccugggcaacauggcaaaauccugccuuuacaaaaaauuagccaggugugguggugugugccuguaguuccagcuacuugggaggcugaggcgggaggaucacuugagccugggaggucgaggugcagugagccaugauggcgccacugcacucuagccugggugacaaagcgagacucugucucaaaaaacaaaacaaaacaaaacaaaacuuggaaaagggaauauuuacacucaaugcagucauucucaguagcacacaauuggcauauugggcuggaugauucuucagucugugggauucuucagugcauuacaagauauuuagcauccuccuuccuuuuaaugucuguacauuagccuacacauuuccaaaggcuacucagcaacagaaucuccccaggagagaacuagaaguaaacuuuuaaggaugugaagaaaacagauguguucuaacaaaauuuauuaagugaaauuaaauggcuaauacuuaagauuccaaaugcuauauauggaagacuauaacaugcaaaaucauaauauuuuaaguacugcuuuccuguuuuaaguguucauuacauuuacucaguaaauccuaauuucccauuuugaagcuucucauuucauugcuucauucacauaucucugauuauauuugauuucuaaauacauuugcuugcucauuacucuuaggcugccugugaugaagucaacaaauaaacauccuuaauuuaaggcaaaaaaauaaaguaaaugcauguuccuuuugacuuccacaugacuuucaguccucccuuaacugaucuuuuacuuugaagacuucuaaaugcuguguuugugaucuggguugaagaucuaaggagaaaagaugagaagugaccaaggcaaagaaagugaugagaagaugaauaaaagagaaccugugcaacugaggucuaaacuggggucuacaaaaauaugauucuuuugcagaucagguucuccagaaagcagagcuuagcgcuuagcaugcaggaugcuuauuaagaaaugucucuagggucaacaaccgugaaggggaaaggacagaaaaggaagcaggaguggguaggagagguggaguugcaaacaaaacuuaggacagccucagcugauaccacagcagcucaggagcuagaauggucccuccaaguuggucuuaauugggaugaaauaaccagguuuuuuacuucccucucuucccacuuaucaauuaguucacugcaugugggcagcaccaccaagccuagggucuugaagagggacugucugcagcugaggugauuugugaaggggcuggcacgugaaggcugucugccuacgacacucccaucagcagagguaucaaguccuccacugaagugguaucugagugggcagcacaguguccaccacagauuccaacuccauauuuuaaagaaguggaaacuuggggacagaaaguaaucagugaguuacaaaucucagucuaaaccccagaauuuaucacuugcugcuucuuacuacuucuaaccaagaaaaaaaugcuaugaucccuauuuuggaaagaaugugcaaggaaauuauuuacuugguuuaaggaauuuucuggaaugggaugaacaaaagcaaaguguccagacaauucuagcuaagauuuuacagagaagacuaaauccucaaauucaucacuauugugaagagcaucauaugucaaauguaauacaccaaaguguuuucguauuuucuguauggcuuuauguaugcucugcaggauauucuguuuguguucccuacauucauguuaccaacauauauuuaugccucucuuuucuaggucuucauugggucucaacaggaaaaauauuaaauuguuaauaaucagggcugguaccaacuagaugagcacacugcaauuagcagccugacuaaccucccugcuucuucauuuucuaauuuuauuugcaagcaguuagauaugauauaauaauuucccauuuaaaaugcuauggauaggaaugggaauauaaguguuuugucuauguacaggaguucuguuuuucuagcacacuucccugccuagccuggacuccagccacacugaccuuucuguucuguauucuugccaugcuccuuuccacacuugucuccccacccucaagucaucacacggcugacuccuuuuccacucccagcugcuggcauuaaggucgucuccucagagguccacauaaucuaaaguaauccuucccugacuaagccaaacuuauuuuauauuaacuuauuaucuugguaacacaauuaccucagaucaucuugcuuauacauuuaucuguuauucauuacuguaucccuaguacauugauuagugccuggcauuuacauauacagcaauuaugcauuugaaagaaugaaugcuggcaaugaguaaauauuuacaauuuauaugauuuuaaauacacaucagaaacacaauaauuacaaaauuacauaucuggagggaaaaucaauucauucuauauuuuugaauaauuuugaaaaaaagaaggaacugcuacucuguguccuaauaccacaauuuguauuaaauaaacucuaagaugucuaucuaucugagccuuccuaaagggcacaaugaugcacgaaagaaugcuuuuguuuuccacaaauacuguaauuuuugagguaacaaaaauacauuggaccaugugugagacagucuucugaauaaaacacuguaagaaaaaaauguugaggccaggcaugguggcucacaucuguaaucccagcacuuugggaggccgaggcacaggcggauaacuggaggucaggaguucgagaccagccuggccaacauggugaaacccugucucuacuaaaaaaacuauaaaaauuagcugggcagguggugcacaccuguaaucccagcuacucaggaggcugaggcaggagaaucacucaaaccugggagguggagguugcaguaagccaauaccgcaccaucgcacuccagccugggugacagagcacaacucugucucaaauuuaaaaaaaggaaagaaagaaggaaaaggaaagaaaagaaaagaaaagaaaagaaagaaagaaagagaaagagagaaagaaagguugaaaacauucuuaagagcaacuucccaauuucacuuuaaaaacaagccuauaaugaagaaagucuguauaagagagacaaacaaaaacaagucauaaauuaggucacuagauuuacaauaauauuuuugcaagaagcuuuuauaauuuuuggcuagcauuuaagcuaggaauauaaaaugucccaggagauucacugaacucaggaauucaucuucugacagggucaaauggaauguccuuuaugauccuggcugaauauuuauccucacacucaaguauggaucauuccauuuaucuaagucuaugcuuuuaaugaaacacuaaauacuucauuuuuaauuaagcaagguaauaaauuuuuauuuuaacuacaucuguuuuaaguuguaacucuauacauguucucuugucuguauuagggaaaauuuuucccauuuaccuaaaauucuuuugagucuucuuucugucaacucuguaguuauucuagaaauuggauucuugguggaaaauaccauguuuacucuaccacaccauuuauuuuuaugaggcuuuaauuccaucgucucucagacaucaucuuuuaggauccuggaagaauuacuggguauaggaaagccuggucuaagaaaguagauuuucuuacagaggccagagacacaguuauaacugggauugaugucagcaucauaaaaacacaagcaggcaggaccucugucuuaguaucugaagaacugauaugacaaaagaaagaugucuuauauaucaugagguauacuagaaaugagcauagauaaauccuagaauuauuuuuaaaaugcauuuggcaggccaaauuuuaucaaucacuaucuaaaauuaauguuaguuucuccaaauuaucaagcaguuguacuagacuauaaugucacuaaugugaaagaauaccuggaaaaauaaaaauagacccuugaauguaggcaagguccuucagcgguaauuucacacaaacaauaauucuauguugcacaauagucaacugauuuuauguuacucacauaggauacuacauauuguaauaauucagggggaaaaucaaacaugauccuuuacugugucuuauuuauuuauuuauuuauuuauuuauuuauuuauuuauuuagagauggaguuucgcucuuguugcccaggcuggaaugcaauggugcaaucucggcucaccgcaaccuucgccucccggguucaagcgauucuccugccucagccucccgaguaccugggauuacaggcaugcgccaccaugcccggcuaauuuuguauguuuaguagagaugggguuucuccauguugguuagucuggucuugaacucccaaccucaggugaucugcccgccucggccucccaaaaugcugggauuacaggcgugagccaccgcaccuggccugccguguuuuauacccauauuuuaguaaccucacaaaguuaaaaaugauuuccaaaugaaaugucagagaauuguuuauuuuacccaguuauacuguaauaaguccuggauggcacugugucuagccucaaggaaacagaacaaguacaaagcuaaguuaaaggcaacuuuauuacaauacuuuuccaaacuucaaaaugcauuuauuacuaaauuuagcucuaauauauaauacacuauauuacaaggcagaaagcauuucuggucauauauuuagcuuccauguauugcccaaaacacagcuuccuaggaauagcagcagugcacaguaguuuacgucacuaaguaaaauauuucucugaugucucacaaucauucauauuguaauuuauuuuauguucuucugauuaaaaaaagcaaagcuaucuuuuccauaucuuauuaguuaauaccuagaaauugucuauauucuaaaaauauccacuagagauauuuccuuugauaggaaauaggauuugcaaauccaaccaaaccaaagaauacuguauuucuccauugcaaauagggguaaaauucucuucuuuccuuuuucaggagccaaaucuggaccuccuccucuucauagagccauccugaauaguaccuacuuuaaaagauaccuaaaucuacaaugcacgcacucuucuuuaauaaagucuuuguuuauucuugccuccuuauauuuacuuauuuagaauaauuuacaaucaacuuugaauguaacauaauaaugaacuuaaaaaguaaucuugggagauguuaauaauauaaagaaaguauuuuccaaauuagacaaggacuuuauuugcucauuauccucugcuucaaccaaccaacuaaccaaauucaacuuaugaaaauaauucguuuuccaagauuacuacccuaaucacaacaaaaacagauacaaauccacaaauagaucuagucccuuaauuuucuauuuuuuuuuuuuuuugguuuuacauuugugagcuaccaacuagcacaaaacauguucaucuuccuuuucucacaacaauaggaaauuguuagaaaaguauagaaaaaguucuauuuuuucauaaggcaaauuuaauacagauuuugcuaugaaguugaaaaaaaaaaaguaaaugaaaagguaugugcuggcugcuuggggggcaaggcucauaaaucacuguacguuaagagcacgugcugaaacuucauggugccagucacguaauuuuguguauuugggacuaaagacgugaagggguaucuuuucccuguggaaauacaaaaaaaaaaaaaaaaaaagagugauagaagagucauaagaaauagaaggaaaaagguucuagacuaauuccaucaugcagaaaacaugccuuauaaaagcuuaauuucacauagcuguuuuauuaaguggcaaaaauuuuaaggguagagcauuuuuagaaauauagcaauuaauccacagaucccucuuggaaacauacauuuuuauguucaaaauaaagugacugcaagacaacacacgagcucauucacagacauauguauuguuagcaaagcaccccgaaauauacccuucugcccacuucuuauuucaauacuagggaaaacaagccuuaaaauccagacaauaucuacuacuuuuaaaaucugcaaguuguacauuuaggucacacaaaaggcaccaaaguuaaaugcuuagcuucuagcugcaaagacgaaaugccauaccauuauacuugacaauuugaaaaacuaaauugagaccauucgugaaaggaaaacauucaauugucaaaaagauagagaacuuacuuugccuucuuuguauuaucggcuucagauucuuucacugaaauauaauaagcaauauccaauauuaguuuguuuuguuuaacuucaaaaucacuuaagcaauuaccugcaccaaucaauaucaaauguucaacuuccauggagccacuaaaacgaauuccauaguagccuguuauuggccauaaacuauaguuaguacaacuacuauuacaucuauacuuuucaauguuacucuccugggcugguguuuuauuuauuuauuuauuuauuuauuuuugagacagagccc

SEQ ID NO: 4 - DNA, 25 bp, Artificial Sequence
  primer
gggtaatactgcaccttcaattgac

SEQ ID NO: 5 - DNA, 20 bp, Artificial Sequence
  primer
ataagaacaatggcatgcgg

SEQ ID NO: 6 - DNA, 22 bp, Artificial Sequence
  primer
gaaaaagtctcaaccggtccta

SEQ ID NO: 7 - DNA, 22 bp, Artificial Sequence
  primer
ggggatttcttgaagatttggg

SEQ ID NO: 8 - DNA, 20 bp, Artificial Sequence
  primer
tgctggaaatgcctgttgtt

SEQ ID NO: 9 - DNA, 21 bp, Artificial Sequence
  primer
attctgcctctaaggaacgtg

SEQ ID NO: 10 - DNA, 20 bp, Artificial Sequence
  primer
ttttggtccccttgcaatct

SEQ ID NO: 11 - DNA, 21 bp, Artificial Sequence
  primer
gaagggatggaaaagcatttc

SEQ ID NO: 12 - DNA, 21 bp, Artificial Sequence
  primer
acggcctttggaagttcagaa

SEQ ID NO: 13 - DNA, 24 bp, Artificial Sequence
  primer
ctcacaaatttcattagacttgga

SEQ ID NO: 14 - DNA, 24 bp, Artificial Sequence
  primer
actttggcccctactaacttctca

SEQ ID NO: 15 - DNA, 24 bp, Artificial Sequence
  primer
gagggctaggtgaagaagtacatt

SEQ ID NO: 16 - DNA, 20 bp, Artificial Sequence
  primer
gaatccaggcacaggttagc

SEQ ID NO: 17 - DNA, 20 bp, Artificial Sequence
  primer
aactgagttcacccccaacc

SEQ ID NO: 18 - DNA, 20 bp, Artificial Sequence
  primer
ctgtgccccagtgtgaagta

SEQ ID NO: 19 - DNA, 20 bp, Artificial Sequence
  primer
gggcccagttcactttctct

SEQ ID NO: 20 - DNA, 20 bp, Artificial Sequence
  primer
tttcttggtgttggggtctg

SEQ ID NO: 21 - DNA, 22 bp, Artificial Sequence
  primer
tctgttgtttaatgccatcacc

SEQ ID NO: 22 - DNA, 75 bp, Artificial Sequence
  probe
cgaaagccatgacctccgatcactctccataacatcttaccaatttatactcccatggcactgccgcaggcattg

SEQ ID NO: 23 - DNA, 85 bp, Artificial Sequence
  probe
tcagtaaaccctaaccaaatgacagggatagagatcattcatcccacctccataaaattggttttgcctttcagcaattcaactt

SEQ ID NO: 24 - DNA, 75 bp, Artificial Sequence
  probe
cgaaagccatgacctccgatcactcaagctgttgtgtgggttcttccctgtgagaatcttaaggatggaaatggt

SEQ ID NO: 25 - DNA, 85 bp, Artificial Sequence
  probe
gagcacaaaccttggcatttaaggaccgacaatgctgagatgttgtggtgcagataaggttgttattgtggaggatgttactaca

SEQ ID NO: 26 - DNA, 75 bp, Artificial Sequence
  probe
cgaaagccatgacctccgatcactccacaaactaaaacagttatgttgagccgggaaaccgtcttcttgtaggct

SEQ ID NO: 27 - DNA, 85 bp, Artificial Sequence
  probe
atcttctagcaccaaataatgcccacatgatttctggctgaaggccatcacttccttcctgtgttccagctacaaacttagaaac

SEQ ID NO: 28 - RNA, 14998 nt, Homo sapiens
guuugccugcacaagcucucucuuugccugcugccauccacgugacuuucuccuccgugccuucugucaugauugugaggucuccccagccacauggaacuguaaguccacuaaaccucuucuuuuguaaauugcccagucucagguaugucuuuaucagcagcgugaaaacagacucauacaacuaacaagccugcuuuagcuuuucacuguucugcccaagcaacuuuccccuucagccaggccaguagauuucucauacagcagggugacccuguucagggccuuuguacuguuuauauuuuuuacuuugugcauacuaugaugucuugacguuuuggaaaccucucuggcuggggagagacuguccuucccagagcuggccaguucuuagaaccagaagggcccagccaggagcacagcucugauaugcaaaccaaccaacccagagcuaugccgucuuuaucuagccuguccaccccaggaagcaguauuucuucaccuauaucaugccagagccugauaacaagcaacuagaaaccaugccuguagcuuacagccugccaaaauuauucaaacuagccaauucuaaacuguucacaugcacuccccugcccugucccugaaagcucaagaaagaccauggcccaggcucuccccucacuccugccccuucaggcuccuacaggggggaccaagacugaugcuuccccauguggcccugcagggcaugacaugcuucccgucucuaagaccugugaguauaauaaccuuggccuuccugagcaccuccucugccuccuccuguggccacaucucacugaccaucacaaaaaacaacauagaauaagccaggcaugguggcuuaugccuguaaucccagcacuuugggaggccgaggcaggcggaucacuugacgucaggaguucgagaccagccuggccaacaugguacaacccugucucuacuaaaaacacaaaaauuagccaggugugguggugggugccuguaaucccagcuacucaggaggcugaagcaggauaaucacuugaacccgggaggcagagguugcagugagccgagauugugccacugcacuccagccugggcaacagagcaagacuccgucucaaaaaauaaaauaaaacagguuuucauuuuaauuuuuaacauauaaaccuccacaggaacauucugacacagacuucagccccaggccuacaucacccuaugauccaaauuauuuuauaguaaaugguaugaaguuuggagaggaagggcaauuuggccaucaugcugugauuuuacugcagguaaaguagaaucaccaugaaagugguggagccuugcuuuuucuuuaauuuuuauaaugccaccacccuacauucuuaggagaaaaggcaaggaguuaaauaaauaagcacagauuuaaguucacauuucugguaauuauccgcaagcuaauaacugagauugcuuaagcucuguaaacaaaacacacaaagcccuuguggcucugcgccagaaucaaaggucugauccaaaacucccccauccccuuguuggcuuugaacaugaaaccaauuuucuacaccaaaucaggccuacuucuuuguaaccauccuuaccaucucucuuauaggaaaauguuuaggacucuuggcggugacucuagaacuaggaaaucaacucucucagcagcacucuaaugagcaaaaacaacugugccucaaaauacaggcugcagugggcagaaaucucuguacugcacuccagccugggugacagaguaaguuucugucucaaaaaaaaaaaaaaaucaaguaacaaagaacagcugucuaaaacuguuaacacugauucaaaauauaauaaccuuuaugccuguuauucagaguuaaaguggguacgcuaauuauaaaucauacacuuuucuaauugugcaauacucugucaaugggauagauuuuaggaaaauuccagcugaaccaguccauucuuugaaucauuaggucaauuacaaacaugccaauccuuagccugaauagcugcauauuuagaaagaccuaaaacauucuucaaaaagcugccauacauggucuguuaaagggcuuacuugguaggaaauaaaucaagagggaauacuccauagugaaucucagggcacuuuuuaaaacaaaucucagggcugagcacaaugguucaugccuauaaucucacugcuuugaggggccaaggcaggaggauugcucaagcccaggaguuugagaccagccugggcaacauggcaagaccuagucccuacaaaaaaauuguuuuauuagcuaggcaugguggaacacgccuguggucccagcuauuccagaggcugaagugggaggaucacuuaagcccaggaagucaaggcugcagugagccaugaucucaccacugcacucgagccugggagacaguacaggacccaucuccaaaaaaaaaaaaaaaaaaagugaacgaagcugcaauaagcugugaucauaccacugcacacuagccuaggcgacagagugagacccugccucaaaaaaaaaaaaagaaaaaaagaaaaaaccaauugauaaaagaguaaccucccaauccccagcuguggaacccaagagcagagaaggcagccacccgccucucuuucccucaaagacaccagagugcaauaggcucuuccucacucauucccaugggaaguagccuucaaccccucucaaaccacagcauauagagaugccuuuauuaauaaaaaaggauacaacccacgggccagccgcaguggcucacgccuguaaucccaacacuuugggaagccaaggcaggcagaucaccugaggccaggaguccgagaccagccuggccaacauggugaaacucugucucuacuaaaagcacaaacauuaaccgagugugguggcacacgccuguagucccagcuacuuaggaggcugaggcaggagaaucgcuugaauccgggaggcaaaaguuggaguuagccgaagucgcaccacugcacuccagacugggcgacagagcgagacucagucucaaaaauaaauaaauacaauaaauaaauaaacaaaguauacaacccacaacucacuuuuuuuucuaaccaggaaucucaacaacaggacuauagggaaacaagaauuauuaaccucauuaauucaaauuaacucuuaagcacccaccacuaacaggcacucuuauagguucuagaccuaggaucauuuuuacagaguucacauuggggaagggagaggggagagggaaaggcaaucacacuaaaaagagggggucagaguaacaguaagauccacacagaaggccaggcacaguggcucacaccuauaauccaagcacuuugggaggccaaggaggguggaucaccugaguucaggaguucgagaccagccuggccaacauggugaaaccuugucucuaccaaaaauacaaaaauuagccgggcaugguggcgggcgccuguaaucucaguuauuugggagccugaggcaggaggaucgccugaacccaggagguggagguugcagugagcugggaccaugccauugcacuccagccuaggcaacaagagugagacuucucaaaaaaaaaaaaaaaauccacaaagaaaguagggagccaugguagugaaugggggcguuuacucuagauggguggucaaggcagaguuucccaggaggacaaagcccccagugacaagagcagggucaacaaguuccaagcaaagagaacagcaagugcugaagccuaaggcuaaugcacuuacagaaaaauccuuaaugacuaaguguguugccucuguuugcgcagagaucacaaaacgugucccgcagaagggagagaaaaagggaccccaggagaggguggcggggcuucagguccuggagaaaggcagcaagcauggcuaacacauuccuugguccacccugcaggguaauacugcaccuucaauugacuuaugaagaaacuagcucagauuggaacaacagccaggacagucaccaccaaggggacuugccccacuggagaagagaccggagugggagagaaacuguuacagccaaucacuuccuuccaggaaaaaaaaagacgcaaauaacaacacaaaaaaacgugaucuaauagacaauccagauauaaagaacacaaacccuuuucccuacaauuuaaccaaaugcuaaaaugcaccaggaaacacaggauacuacagagauaaagauuuggccagcagauuaucaaccccaggaaaagaaauccauuucuguagcaugcuaugcuguuggaggcacacgucacacuucuuaauucaaggacagaaaagcacaaacuaaaaugcacacuggaguaugacucccccuucucuaucugcuaaagcgcuucgcaugugacuuucaaucucuaagcugggaaucaaucaggcagcccagccuccuauauuuucuaguuguauugcuuugagcaucaaauuuaaccucugugcaccucaauuucuucagcuggagacaaagaaaguggcugcacuguggcagaguguuugcaaauguuaaucaugguuaugguagcauucuaccuugucuuacagaugguugccuaaaaagguaggaaacuguucacgaaaaagucucaaccgguccuagagugaucagaaugacagcgcugaaauaaggagggcaguaccucuuauuccagagaaguggggagcagagaggaagauggaguggaaaggggcgaggcaaggcccuccugaaauaccucaacccaaaucuucaagaaauccccaaguccucacagugcuuuuuguggauuuuuguggaaaccgguaaaaggggcugauuugcugaccccaguggguagaaaacagagacugucaagagaacagaagagaaggcagaaaggggauggggaagugggguucgccauguucacgagcuccuggagccacagggccccccaggaacaacagagcugagacuggguggccuuguuucuggcccaauucccugggaccaccacccuggaaggacacuugagcccuacucaaggacccaccacaacaucucagcauugucgguccuuaaaugccaagguuugugcucaccauuuccauccuuaagauucucacagggaagaacccacacaacagcuuugucccugcgacucugggugaguuucugacacuggccugggcucccguuuugcccgucaccaacuucucacaguauaacuauccaagagaaguagcauuugaugucagggaaucaaaagggcuaaggcucgggaggccaaggcaggugaaucacuuguaaucaggaguucgagaccacccugaccaacauagugaaaccccaucucuacuaacaauacaaaaaauuaaccaggugugguggcaggugccuguaaucccagcuacucaggaggcugagguaggaggaucacuugaacccaggaggcagagguugugguacgccaagaucaugacacugcacuccagccugggugacagagugagacuccaucucaaaaaaaaaaaaaaaaaaggccuaaggcuuacucagggucaucuaugguuugccaagcagaguucaauccaaucaacaaaacccucacugcccacuuuuccaaaaccugauauagucagucacuaguagcugccauuugcuguuuugccuucuauuaaaaaaaaaaaaaaaaaaaaaaccauccaauucccagaagucaaaggcaauuccuucccuuuucugugucugugagacggcucucugugggauuccuuccugcugcaccucauccuuccuggaagggacucucagaaaaugugccuccuccuuacaggaaaggaauuccucccgaggaacuguggacaugaggcagcuuuguggaauuucccuuccacagacuucagccuauuuucugggcaaacuuagcagaaaugaccaaaauccacucgagagagacagcaacaaggagaggggaagagauccuauuuuggcuucuucaaauggaccaauuuucucuuuaaaauauuuauuuuaaggaaaaacaaggacuacauuauauuuuaaagggggagaaggaagaggauuuugaguugcuaagagaacagagccaucucuuuucuuuuaauacuggaugcuaagaacuugcacaggguuuucccaaaagccagauaguucagguaauuugcauguggcuguaaaagagaaaguucaggccaggcacuguggcaacauggugaaaccccaucucuacuaaaaauacaaaaauuagcugggugugguggugcaagccuguaaucccagcuacuugggaggcugaggcaggggaaucacuugaaucugggagguggacguugcagugagccaagaucacaccacugcacuccugggcgacagagacucucucaaaaaaaaaaaaaaaaaaaaaaaaagagagagagaaaguugagagcauaagucugagugauucugccugaaagaacagaacagcuuacacuggacaggcuuuugcccaagacacaccucaucuggcauugagaaacgaacaccaacaauaaugagugacagaauguuaagaacaauaauuauacagacuuggaccucccagacugcuugucuguaguaauucagaaggaagcaaaguauucugaaguguuuaugaagauugguuccacaucugaagaauuuuaguaaucuuacccaaacucaacccaagaaauaugaggaaaagagggaguaguucuauaauaagcagguggaauagagggucaaacuccuuggcgaguucagucuaacagaggacacauuacugcccuacaaagcagcucuggaaaaugacaaccaguggcacuaacuccaggcaauaucuuugugaggcuugugggcuguaauauagucugcucaggcugccauaaaaaaaaaaucacaaucaggccaggcacuauggcucacaccuguaaucccagcacuuugggaggccaaggagggcagaucaccugagguuaggaguucgagaccaggcuggccaacauggcaaaaccccgucucuacuaaaaauacaaaaauuacccggaugugguagcacgcaccgguagucccagcuacucgucaggcugaggcaggagaauugcuugaaccugggagggguggagguugcagugggcugagaucacaccacuguacuccagccuggacaacagagccacugcacuccagccugggcaacagagagagacuccaucuuaaaaaaaaaaaaaaaaaauccccgucuucaaauacaaucacauucugaaguacuagagaucaggacuccaacaugcgaacuuuggggggacauaauucagcccguaauaugggugauaaaggagcagguccagacuuagagaaacaaggacucccugggucaagcucuggaagugaaaggcuucuucaaagacaggaaagucccacugaagguggcuuuggaagugcacccuugcagcacagcaaccuucuugauuugcucaagagcagggguucugacaggucucaaagcacuuuguuuaaugaaacagaaucgaaaauaccagugugucacauguuuccuaagacaggacacuguuucuugaaacuuuuacuaaaguauguuuuucuuuuauuuuuuuauuuuuuauuuuuuuugagaaggagucuugcucugucgcccaggcuggagugcaguggcacaaucucgacucacugcaaccucugccucccggguucaaacaauucuccugccucagccuccugaguagcugggauuacaggugcacgccaucaugccuggcuaauuuuuguauuuuuaguagagacaggguuucaccauguugaccagucugcucucaaacuccugaccuugugaucugcccaccuuggccucccaaagugcugggauuacaggugugagccaccguguccagccuaaaguauguuaaauauguuaugugugagagagagaaagagaauauaucucuuggucauuacauauguaaaacuuguagaacgugaucaaaauguuuaaaaacuggccaggcacaguggcucaagccuguaucccagcacuuugggaucccaaggcagguggaucacaaggucaggaaucaagaccauccuggucaacacagugaaaccccaucucuacuaaaaauacaaaaaauuagccuggcgcgguggcacgcaccuguagucccagcuacuugggaggcuggggcaggagaaucgcuugaaccugggaggcggaggcugcagugagcugagauagcgccacugcacuccugccugggcaacagagcaagacuccaacucaaaaaaaaaaaaaaaaguuuaaaaacugguccuguggcagugggucucaaacaucagcaugcaucagaaucacuuggaggacugauuaaaacacaggcugcugcugaugcugcuaauccuagagccaccuugaaguuuuucugugguuuuuuuuuuuuuuuuuuuuuuuuuuuugagacagggucuugcugucucccaggcuggagugcaguggugcaaugauaccucacugcagccuugaacuccugggcucaagcaaugcccccaucucagccucccaagcaacuaggacuguagcuagcuccuaacucauucccuccauuccugcccuccccuaacuuuauauugcagccagaaggauauuuuuucugaagagcaucucaugcugaguggcagcagaaugcagagauuaagagagggggccaggcacaguggcucacaccucuaaucccagcacuuugggaggccgagguggguggaucacccgaggucaggaguucaagaccagccuagccaacauggagaaaccccgucucuaugggcgugguggugcauaccuguaaucccagcuacuagggaggcugaggcaggagaaucacuugaacccaggaggcagagguugccgugagccgagaucucgucauugcacuccagccugggcaacaagagcgaaacuccgucucaaaaaaaaaaaaagaguggcgccaggugcaguggcucacgccuguaaucccagcacuuugggaggccgaggcaggcaaaucaucugaggucgggaguucgagaccagccugaccaacauagagaaaccccgucucuacuaaaaauacaaaaaaauuagccgagcgugguggcaugugucguaugccuguaaucccagcuacuggggaggcugaggcaggagaauuacuugaaccugggaggcagguuuuuuguuuuuuguuuugguuuuguuugagauuuugguuguuguuuuuugggguuuuuuugagacagggucucacuguguuguccaggcuggagugcaggggugcgaucaugugucacugcagccacaaccuccugggcucaggugauccucccaccucagccuuccugaguaacuguuacuacaggugugcaccaccaugacuggcuaauuuuuguauuuuuuguagagacaagaguuucaccauguugcacaggcuguucacaaacuccuaggcucaagcuauccaccugccucagccucccaaagugcugggauuauaggcaugagccaccuugcccagccaacugagccacuuugugccaggacuaggucagguacuugggacauauacaggcaaggacacugcccacagggaacugacagugucuagcagcuacacagaugacacggccuuuucggcagacaccccuguccccugugcuuggcaaacagcagagacucaaaaauguauaugguuugacucccacuggaacauaagccccuuagggcagggaccagggucacuguauucccagcaccuauaaucaagagguacaugacgcugcagcugagagcagggacucugugcuaaguuaccuaccugguuugaaccuaggcucugccacucaaaggcugugugaccgugggaaauaauaguacccaccccaaagguuuguuauuaguauuuaauaauauuuauugguaagguacuuagaauagccccccugccuacaggaagcaguacauaauuauaaugucuaaauauacaguaguuccaggccaggcaugguggcucaugucuguauuccuagcagaaugagaggccgaggaaggagaaucauuugaguccaggaguuugagaccagccuggauaacauagugagaucucgucucuacaaaaaauaaacaaaaguaagccagacaugguggcacaugccugcaaucccagcuacucaaaaggcugugauuagagaaucacuugagcccaggagguagaggcugcaaugagcugagaucacaccacuguacuccagccugggcaacagagugagacuccgucucuuaaauaaauaaauaaauaggggccaggcgugguagcuuacaccuguaaucccagcacauugggaggcugaggcgggaggaucacuugaggacaggaguucaagaccaaccugaccaacauggugaaaccccaucucuacuaaaacacaacaaucagcugggcaugguggcgggugccuguaaucccagcuauuuugggggcugaggcaggagaaucacuugaacccaggaggcagagguugcagugagccaagaucacacuacuacacuccagccugggcaacagagcgggacuccaucucaaauaaauaaauaaauagcaguuccauauccucugaauaauggacuagaaguugguuaauggauggguuuucagggagaauuuuagcagccgagcugagagguagguacucuagccaggguauauaacaugagcuaagggaacaggacagcaaauauaaggugugaucaggggaaaggcaucauauauugacuguccaggaacaauggauaucugagaauguaggaaagcuaaguuaggaccaggaaaaggagaguauuaaaugccaaagaagaugccaccaaagguucuggaacagaaagcagcagagcuaugccuuguaaagauaaagccugcaguguauugacuggguuggaaucuggaaucauugcgugccuccagucaagacguggcaacugaccaacuaccuuuuaucacugaccagacucuucccuacgugcagauaacugaggacgcaagccuuaguuacugggugugguggugcuauuaacggaaaaagggaagucagccagcaaaggcugacuggggcaauuucagccuuguugcucugcugugaaacugcuuuccagaaaaacuuuuuuuuuuuuuugagacggagucucacucugucacccaggcuggagugcaguggcacaauuucggcucacugcaaccuccgccucccagguucaagcgauucuccugucucagccuccccagaagcugggauuacaggcgcccaccaccacacucagcuaauuuuuguauuuuuaguaaagucaggguuccacuauguuggccaggcugaucuccaacuccugaccucaggugauccgccugccucggccucccaaagugcugagauuacaggcaugagccacagugcccagcccaggaaaacuucuuaaagauguaauaauaacgcaccuuauuauuuuccaaguacuuccauagauaguggcccacuucugaguaugaauuaaauuucccuuuguggccuuuguuuuuuuaauagaucuucauuucuggggggacuuacaaacaggugacuucuccauucaccagagaugggacauuauacacugaccuacuuccauauucacuuucccugcuugauuggaggugagggagaaaaauuaguguauaaggccgggcgcggaggcucacgccuguaaucccaggacuuuagaaggcugaggcagguggaucaccugaggucaggaguucgagaccagccuggccaacauggugaaaccccuugucuacuaaaaauacaaaaauuagccaggugugguggcacacgccuguaaucccaguuacucaggaggcugaggcaggagaauugcuugaaccccgaggcagagguugcagugagcugaguccaugccauugcacucuagccugggcaacaagagugaaacucugucucaaaaaaaaaaaaaaaagaaaagaaaagaaaaauaaguguauagaacuuuauuguuucauugaccgggcacgauggcucacgccuguaacucuagcacuuugggaggccaaggugggcagauuggcugagcucagcaguuugaaaccagccugggcaacauggugaaaccuaucucuaauaaaauacaaaaaauuagcccgguaugguagcacaugccuauaguuucagcuaccugggaggcugaggcaugagaaucacuugaacccagaagguugcagugagccaagauugugccacugcacuccagccugggcaacagagcaaaacucugucuccaaaaaaagagaacuggccagggaugguggcucacgccucuaaucccagcacuuugggaggcuaaggugggcagaucacgaggucaggaguucaagaccagccuggccaacauggugaaaccccgucucuacuaaaaauacaaaaauuagcugggcaugguggcaggcaccuguaaucccagcuacucaggaggcugagacagcagaaucguuugaaucugggagguagaaguugcagugagccaagaucacgccauugcacuccagccuggguggaagggcaugacuucgucucaaaaaaaaaaaaacuuuuuccuuaaaauacaccucuuggagccagucccagauacuugggaggaucaggaaggaggaccucuugaggccauaaauuugaggauguagugugccugugaauagccacuacacucuagccugggcaacacagaaagaccucauaaauaaauaaauaggccagaugagguggcucacaccuguaaucucagcacuuugggaggccaaggcuggcagaucacgaaguuaggagaucaagaccauccuggcuaacacagugaaaccccgucucuacuaaaacuacaaaaaauuagccaggcaugguggcaugcaccuauagucccagcuacucgggaggcugaggcacgagaaucacuugaacccgggagacggagguugcagugagccgaaaucgcaccacugcacuccagccugggcaacggagugagacucugccucacacaaauaauaaauaaauaaauaaauauuuuauaauguauuuuuaaaauauaguucuuaaaacaugccaugaguagguuuaauuuuucuaaaucauagaauaggauacugaagcccagaguggucaugugucuugccuaaggucacacacagcuaauauauacaagaucaugggucuucuggcuccagucugaggcugucacaauaacuagcuucucacuugguccuaccuaagacacaggugucagcaacucuaaauucaagaaauugagauuaagacuaaaguaauagcuuauaaacauuaauuaagcauauuaauugcaagauguuaaaccagucacugagagauuaagacauugaagauacugguuucugccuuaauagagcuaacaauuuaguaaaaaagucgcaaaugagucaagacuuaaauaagacuuaaagacuuaagacaauaaaaaaggcaugauaaaaggguaaauuuaggcuggacgcaguggcucaugccuguaaucccagcacuuugggaggcugaggugggcggaucaccugaggucaggaguucaagaccagccuggccaacauggcaaagucccaucucuacuaaaaauacaaaaauuagccaggugucauggcacaagccuguaauuccagcuacucgggaggcugaggcaggagaacugcuugaacccagaagguggagguugcucaagagcaagacucugacucaaaaaaaaaaggaugggggauaaauuuagcaagaacccucaucaucccauuuaccuacagccuaauauguacuaggaacuuacauauacaauaauagguaaaaucuauuaagaauguauuauguuuuauaguugaacaaauugugucgcuuaaucaacuuagaauucauguaaaugccacauuugagcucuguuacauugagagcucuccaggcccagccuaccauguuaagccauugauaaaccucccugcuacuauuuaaaacaagauggauaccuuucuacugucuuuaaagaggaagaguacacacuguuggacuguaugcacucuccgucucucaagacaguaaucugagggauuacuucaauagcagaaguuaaaagaagcggucaguuggguuuauuacauaaucucaaucacauccugaccaauaaaaugcaccaauaaagaucagcaaauaaaagcaagugauuagaagagcaauuuuaugcucacgagcucugaauucacaagugugagauuuccugucaaguacucucaugagcucaauuaaucucugacccagggugagaaaauaaacauagugacgcagaggcugcagcucaugcuuuggaauuugucagacuugacucaguucccaacuccaucauguccugccugggugacccucauuaugcuaccuggacucucucaguuuccuucaccuguauguaacacaagagcagagccacccucaacaagggguuacuauaaaaaucgaagaacccggcaucucuaacaacaucccuggcacggagcaaucccccuucucccucagccacuggcugagcacccacagaggggaguaaguccuauugugguaaugguucucaugcuuugagaucagacaaguccuuugagaggaaccugaaaaaggggcagcgaaccuuuggccagaaaaauggaaauaugcacagaaauauuuuugacuuuggcaagcaguugaucagugguucuugggcuucagugcccaucagaaacguagagaaccuuuuuaaaauauugaugugcggggcgcgguggcucacaccuguaauc

SEQ ID NO: 29 - RNA, 14985 nt, Homo sapiens
cugcaggcuccgccccccgggguucacgcgauaucugaugcuuuuaaaaauaagaguuugccugcacaagcucucucuuugccugcugccauccacgugacuuucuccuccgugccuucugucaugauugugaggucuccccagccacauggaacuguaaguccacuaaaccucuucuuuuguaaauugcccagucucagguaugucuuuaucagcagcgugaaaacagacucauacaacuaacaagccugcuuuagcuuuucacuguucugcccaagcaacuuuccccuucagccaggccaguagauuucucauacagcagggugacccuguucagggccuuuguacuguuuauauuuuuuacuuugugcauacuaugaugucuugacguuuuggaaaccucucuggcuggggagagacuguccuucccagagcuggccaguucuuagaaccagaagggcccagccaggagcacagcucugauaugcaaaccaaccaacccagagcuaugccgucuuuaucuagccuguccaccccaggaagcaguauuucuucaccuauaucaugccagagccugauaacaagcaacuagaaaccaugccuguagcuuacagccugccaaaauuauucaaacuagccaauucuaaacuguucacaugcacuccccugcccugucccugaaagcucaagaaagaccauggcccaggcucuccccucacuccugccccuucaggcuccuacaggggggaccaagacugaugcuuccccauguggcccugcagggcaugacaugcuucccgucucuaagaccugugaguauaauaaccuuggccuuccugagcaccuccucugccuccuccuguggccacaucucacugaccaucacaaaaaacaacauagaauaagccaggcaugguggcuuaugccuguaaucccagcacuuugggaggccgaggcaggcggaucacuugacgucaggaguucgagaccagccuggccaacaugguacaacccugucucuacuaaaaacacaaaaauuagccaggugugguggugggugccuguaaucccagcuacucaggaggcugaagcaggauaaucacuugaacccgggaggcagagguugcagugagccgagauugugccacugcacuccagccugggcaacagagcaagacuccgucucaaaaaauaaaauaaaacagguuuucauuuuaauuuuuaacauauaaaccuccacaggaacauucugacacagacuucagccccaggccuacaucacccuaugauccaaauuauuuuauaguaaaugguaugaaguuuggagaggaagggcaauuuggccaucaugcugugauuuuacugcagguaaaguagaaucaccaugaaagugguggagccuugcuuuuucuuuaauuuuuauaaugccaccacccuacauucuuaggagaaaaggcaaggaguuaaauaaauaagcacagauuuaaguucacauuucugguaauuauccgcaagcuaauaacugagauugcuuaagcucuguaaacaaaacacacaaagcccuuguggcucugcgccagaaucaaaggucugauccaaaacucccccauccccuuguuggcuuugaacaugaaaccaauuuucuacaccaaaucaggccuacuucuuuguaaccauccuuaccaucucucuuauaggaaaauguuuaggacucuuggcggugacucuagaacuaggaaaucaacucucucagcagcacucuaaugagcaaaaacaacugugccucaaaauacaggcugcagugggcagaaaucucuguacugcacuccagccugggugacagaguaaguuucugucucaaaaaaaaaaaaaaaucaaguaacaaagaacagcugucuaaaacuguuaacacugauucaaaauauaauaaccuuuaugccuguuauucagaguuaaaguggguacgcuaauuauaaaucauacacuuuucuaauugugcaauacucugucaaugggauagauuuuaggaaaauuccagcugaaccaguccauucuuugaaucauuaggucaauuacaaacaugccaauccuuagccugaauagcugcauauuuagaaagaccuaaaacauucuucaaaaagcugccauacauggucuguuaaagggcuuacuugguaggaaauaaaucaagagggaauacuccauagugaaucucagggcacuuuuuaaaacaaaucucagggcugagcacaaugguucaugccuauaaucucacugcuuugaggggccaaggcaggaggauugcucaagcccaggaguuugagaccagccugggcaacauggcaagaccuagucccuacaaaaaaauuguuuuauuagcuaggcaugguggaacacgccuguggucccagcuauuccagaggcugaagugggaggaucacuuaagcccaggaagucaaggcugcagugagccaugaucucaccacugcacucgagccugggagacaguacaggacccaucuccaaaaaaaaaaaaaaaaaaagugaacgaagcugcaauaagcugugaucauaccacugcacacuagccuaggcgacagagugagacccugccucaaaaaaaaaaaaagaaaaaaagaaaaaaccaauugauaaaagaguaaccucccaauccccagcuguggaacccaagagcagagaaggcagccacccgccucucuuucccucaaagacaccagagugcaauaggcucuuccucacucauucccaugggaaguagccuucaaccccucucaaaccacagcauauagagaugccuuuauuaauaaaaaaggauacaacccacgggccagccgcaguggcucacgccuguaaucccaacacuuugggaagccaaggcaggcagaucaccugaggccaggaguccgagaccagccuggccaacauggugaaacucugucucuacuaaaagcacaaacauuaaccgagugugguggcacacgccuguagucccagcuacuuaggaggcugaggcaggagaaucgcuugaauccgggaggcaaaaguuggaguuagccgaagucgcaccacugcacuccagacugggcgacagagcgagacucagucucaaaaauaaauaaauacaauaaauaaauaaacaaaguauacaacccacaacucacuuuuuuuucuaaccaggaaucucaacaacaggacuauagggaaacaagaauuauuaaccucauuaauucaaauuaacucuuaagcacccaccacuaacaggcacucuuauagguucuagaccuaggaucauuuuuacagaguucacauuggggaagggagaggggagagggaaaggcaaucacacuaaaaagagggggucagaguaacaguaagauccacacagaaggccaggcacaguggcucacaccuauaauccaagcacuuugggaggccaaggaggguggaucaccugaguucaggaguucgagaccagccuggccaacauggugaaaccuugucucuaccaaaaauacaaaaauuagccgggcaugguggcgggcgccuguaaucucaguuauuugggagccugaggcaggaggaucgccugaacccaggagguggagguugcagugagcugggaccaugccauugcacuccagccuaggcaacaagagugagacuucucaaaaaaaaaaaaaaaauccacaaagaaaguagggagccaugguagugaaugggggcguuuacucuagauggguggucaaggcagaguuucccaggaggacaaagcccccagugacaagagcagggucaacaaguuccaagcaaagagaacagcaagugcugaagccuaaggcuaaugcacuuacagaaaaauccuuaaugacuaaguguguugccucuguuugcgcagagaucacaaaacgugucccgcagaagggagagaaaaagggaccccaggagaggguggcggggcuucagguccuggagaaaggcagcaagcauggcuaacacauuccuugguccacccugcaggguaauacugcaccuucaauugacuuaugaagaaacuagcucagauuggaacaacagccaggacagucaccaccaaggggacuugccccacuggagaaggcaagucugacccccuaccccaccgcggucaacccagggacggaggagagaacgccgcaugccauuguucuuauguauaaagcacagguuacagguuauuucugaagaugucugcucaaguggagaugcuaccagagaccggagugggagagaaacuguuacagccaaucacuuccuuccaggaaaaaaaaagacgcaaauaacaacacaaaaaaacgugaucuaauagacaauccagauauaaagaacacaaacccuuuucccuacaauuuaaccaaaugcuaaaaugcaccaggaaacacaggauacuacagagauaaagauuuggccagcagauuaucaaccccaggaaaagaaauccauuucuguagcaugcuaugcuguuggaggcacacgucacacuucuuaauucaaggacagaaaagcacaaacuaaaaugcacacuggaguaugacucccccuucucuaucugcuaaagcgcuucgcaugugacuuucaaucucuaagcugggaaucaaucaggcagcccagccuccuauauuuucuaguuguauugcuuugagcaucaaauuuaaccucugugcaccucaauuucuucagcuggagacaaagaaaguggcugcacuguggcagaguguuugcaaauguuaaucaugguuaugguagcauucuaccuugucuuacagaugguugccuaaaaagguaggaaacuguucacgaaaaagucucaaccgguccuagagugaucagaaugacagcgcugaaauaaggagggcaguaccucuuauuccagagaaguggggagcagagaggaagauggaguggaaaggggcgaggcaaggcccuccugaaauaccucaacccaaaucuucaagaaauccccaaguccucacagugcuuuuuguggauuuuuguggaaaccgguaaaaggggcugauuugcugaccccaguggguagaaaacagagacugucaagagaacagaagagaaggcagaaaggggauggggaagugggguucgccauguucacgagcuccuggagccacagggccccccaggaacaacagagcugagacuggguggccuuguuucuggcccaauucccugggaccaccacccuggaaggacacuugagcccuacucaaggacccaccacaacaucucagcauugucgguccuuaaaugccaagguuugugcucaccauuuccauccuuaagauucucacagggaagaacccacacaacagcuuugucccugcgacucugggugaguuucugacacuggccugggcucccguuuugcccgucaccaacuucucacaguauaacuauccaagagaaguagcauuugaugucagggaaucaaaagggcuaaggcucgggaggccaaggcaggugaaucacuuguaaucaggaguucgagaccacccugaccaacauagugaaaccccaucucuacuaacaauacaaaaaauuaaccaggugugguggcaggugccuguaaucccagcuacucaggaggcugagguaggaggaucacuugaacccaggaggcagagguugugguacgccaagaucaugacacugcacuccagccugggugacagagugagacuccaucucaaaaaaaaaaaaaaaaaaggccuaaggcuuacucagggucaucuaugguuugccaagcagaguucaauccaaucaacaaaacccucacugcccacuuuuccaaaaccugauauagucagucacuaguagcugccauuugcuguuuugccuucuauuaaaaaaaaaaaaaaaaaaaaaaccauccaauucccagaagucaaaggcaauuccuucccuuuucugugucugugagacggcucucugugggauuccuuccugcugcaccucauccuuccuggaagggacucucagaaaaugugccuccuccuuacaggaaaggaauuccucccgaggaacuguggacaugaggcagcuuuguggaauuucccuuccacagacuucagccuauuuucugggcaaacuuagcagaaaugaccaaaauccacucgagagagacagcaacaaggagaggggaagagauccuauuuuggcuucuucaaauggaccaauuuucucuuuaaaauauuuauuuuaaggaaaaacaaggacuacauuauauuuuaaagggggagaaggaagaggauuuugaguugcuaagagaacagagccaucucuuuucuuuuaauacuggaugcuaagaacuugcacaggguuuucccaaaagccagauaguucagguaauuugcauguggcuguaaaagagaaaguucaggccaggcacuguggcaacauggugaaaccccaucucuacuaaaaauacaaaaauuagcugggugugguggugcaagccuguaaucccagcuacuugggaggcugaggcaggggaaucacuugaaucugggagguggacguugcagugagccaagaucacaccacugcacuccugggcgacagagacucucucaaaaaaaaaaaaaaaaaaaaaaaaagagagagagaaaguugagagcauaagucugagugauucugccugaaagaacagaacagcuuacacuggacaggcuuuugcccaagacacaccucaucuggcauugagaaacgaacaccaacaauaaugagugacagaauguuaagaacaauaauuauacagacuuggaccucccagacugcuugucuguaguaauucagaaggaagcaaaguauucugaaguguuuaugaagauugguuccacaucugaagaauuuuaguaaucuuacccaaacucaacccaagaaauaugaggaaaagagggaguaguucuauaauaagcagguggaauagagggucaaacuccuuggcgaguucagucuaacagaggacacauuacugcccuacaaagcagcucuggaaaaugacaaccaguggcacuaacuccaggcaauaucuuugugaggcuugugggcuguaauauagucugcucaggcugccauaaaaaaaaaaucacaaucaggccaggcacuauggcucacaccuguaaucccagcacuuugggaggccaaggagggcagaucaccugagguuaggaguucgagaccaggcuggccaacauggcaaaaccccgucucuacuaaaaauacaaaaauuacccggaugugguagcacgcaccgguagucccagcuacucgucaggcugaggcaggagaauugcuugaaccugggagggguggagguugcagugggcugagaucacaccacuguacuccagccuggacaacagagccacugcacuccagccugggcaacagagagagacuccaucuuaaaaaaaaaaaaaaaaaauccccgucuucaaauacaaucacauucugaaguacuagagaucaggacuccaacaugcgaacuuuggggggacauaauucagcccguaauaugggugauaaaggagcagguccagacuuagagaaacaaggacucccugggucaagcucuggaagugaaaggcuucuucaaagacaggaaagucccacugaagguggcuuuggaagugcacccuugcagcacagcaaccuucuugauuugcucaagagcagggguucugacaggucucaaagcacuuuguuuaaugaaacagaaucgaaaauaccagugugucacauguuuccuaagacaggacacuguuucuugaaacuuuuacuaaaguauguuuuucuuuuauuuuuuuauuuuuuauuuuuuuugagaaggagucuugcucugucgcccaggcuggagugcaguggcacaaucucgacucacugcaaccucugccucccggguucaaacaauucuccugccucagccuccugaguagcugggauuacaggugcacgccaucaugccuggcuaauuuuuguauuuuuaguagagacaggguuucaccauguugaccagucugcucucaaacuccugaccuugugaucugcccaccuuggccucccaaagugcugggauuacaggugugagccaccguguccagccuaaaguauguuaaauauguuaugugugagagagagaaagagaauauaucucuuggucauuacauauguaaaacuuguagaacgugaucaaaauguuuaaaaacuggccaggcacaguggcucaagccuguaucccagcacuuugggaucccaaggcagguggaucacaaggucaggaaucaagaccauccuggucaacacagugaaaccccaucucuacuaaaaauacaaaaaauuagccuggcgcgguggcacgcaccuguagucccagcuacuugggaggcuggggcaggagaaucgcuugaaccugggaggcggaggcugcagugagcugagauagcgccacugcacuccugccugggcaacagagcaagacuccaacucaaaaaaaaaaaaaaaaguuuaaaaacugguccuguggcagugggucucaaacaucagcaugcaucagaaucacuuggaggacugauuaaaacacaggcugcugcugaugcugcuaauccuagagccaccuugaaguuuuucugugguuuuuuuuuuuuuuuuuuuuuuuuuuuugagacagggucuugcugucucccaggcuggagugcaguggugcaaugauaccucacugcagccuugaacuccugggcucaagcaaugcccccaucucagccucccaagcaacuaggacuguagcuagcuccuaacucauucccuccauuccugcccuccccuaacuuuauauugcagccagaaggauauuuuuucugaagagcaucucaugcugaguggcagcagaaugcagagauuaagagagggggccaggcacaguggcucacaccucuaaucccagcacuuugggaggccgagguggguggaucacccgaggucaggaguucaagaccagccuagccaacauggagaaaccccgucucuaugggcgugguggugcauaccuguaaucccagcuacuagggaggcugaggcaggagaaucacuugaacccaggaggcagagguugccgugagccgagaucucgucauugcacuccagccugggcaacaagagcgaaacuccgucucaaaaaaaaaaaaagaguggcgccaggugcaguggcucacgccuguaaucccagcacuuugggaggccgaggcaggcaaaucaucugaggucgggaguucgagaccagccugaccaacauagagaaaccccgucucuacuaaaaauacaaaaaaauuagccgagcgugguggcaugugucguaugccuguaaucccagcuacuggggaggcugaggcaggagaauuacuugaaccugggaggcagguuuuuuguuuuuuguuuugguuuuguuugagauuuugguuguuguuuuuugggguuuuuuugagacagggucucacuguguuguccaggcuggagugcaggggugcgaucaugugucacugcagccacaaccuccugggcucaggugauccucccaccucagccuuccugaguaacuguuacuacaggugugcaccaccaugacuggcuaauuuuuguauuuuuuguagagacaagaguuucaccauguugcacaggcuguucacaaacuccuaggcucaagcuauccaccugccucagccucccaaagugcugggauuauaggcaugagccaccuugcccagccaacugagccacuuugugccaggacuaggucagguacuugggacauauacaggcaaggacacugcccacagggaacugacagugucuagcagcuacacagaugacacggccuuuucggcagacaccccuguccccugugcuuggcaaacagcagagacucaaaaauguauaugguuugacucccacuggaacauaagccccuuagggcagggaccagggucacuguauucccagcaccuauaaucaagagguacaugacgcugcagcugagagcagggacucugugcuaaguuaccuaccugguuugaaccuaggcucugccacucaaaggcugugugaccgugggaaauaauaguacccaccccaaagguuuguuauuaguauuuaauaauauuuauugguaagguacuuagaauagccccccugccuacaggaagcaguacauaauuauaaugucuaaauauacaguaguuccaggccaggcaugguggcucaugucuguauuccuagcagaaugagaggccgaggaaggagaaucauuugaguccaggaguuugagaccagccuggauaacauagugagaucucgucucuacaaaaaauaaacaaaaguaagccagacaugguggcacaugccugcaaucccagcuacucaaaaggcugugauuagagaaucacuugagcccaggagguagaggcugcaaugagcugagaucacaccacuguacuccagccugggcaacagagugagacuccgucucuuaaauaaauaaauaaauaggggccaggcgugguagcuuacaccuguaaucccagcacauugggaggcugaggcgggaggaucacuugaggacaggaguucaagaccaaccugaccaacauggugaaaccccaucucuacuaaaacacaacaaucagcugggcaugguggcgggugccuguaaucccagcuauuuugggggcugaggcaggagaaucacuugaacccaggaggcagagguugcagugagccaagaucacacuacuacacuccagccugggcaacagagcgggacuccaucucaaauaaauaaauaaauagcaguuccauauccucugaauaauggacuagaaguugguuaauggauggguuuucagggagaauuuuagcagccgagcugagagguagguacucuagccaggguauauaacaugagcuaagggaacaggacagcaaauauaaggugugaucaggggaaaggcaucauauauugacuguccaggaacaauggauaucugagaauguaggaaagcuaaguuaggaccaggaaaaggagaguauuaaaugccaaagaagaugccaccaaagguucuggaacagaaagcagcagagcuaugccuuguaaagauaaagccugcaguguauugacuggguuggaaucuggaaucauugcgugccuccagucaagacguggcaacugaccaacuaccuuuuaucacugaccagacucuucccuacgugcagauaacugaggacgcaagccuuaguuacugggugugguggugcuauuaacggaaaaagggaagucagccagcaaaggcugacuggggcaauuucagccuuguugcucugcugugaaacugcuuuccagaaaaacuuuuuuuuuuuuuugagacggagucucacucugucacccaggcuggagugcaguggcacaauuucggcucacugcaaccuccgccucccagguucaagcgauucuccugucucagccuccccagaagcugggauuacaggcgcccaccaccacacucagcuaauuuuuguauuuuuaguaaagucaggguuccacuauguuggccaggcugaucuccaacuccugaccucaggugauccgccugccucggccucccaaagugcugagauuacaggcaugagccacagugcccagcccaggaaaacuucuuaaagauguaauaauaacgcaccuuauuauuuuccaaguacuuccauagauaguggcccacuucugaguaugaauuaaauuucccuuuguggccuuuguuuuuuuaauagaucuucauuucuggggggacuuacaaacaggugacuucuccauucaccagagaugggacauuauacacugaccuacuuccauauucacuuucccugcuugauuggaggugagggagaaaaauuaguguauaaggccgggcgcggaggcucacgccuguaaucccaggacuuuagaaggcugaggcagguggaucaccugaggucaggaguucgagaccagccuggccaacauggugaaaccccuugucuacuaaaaauacaaaaauuagccaggugugguggcacacgccuguaaucccaguuacucaggaggcugaggcaggagaauugcuugaaccccgaggcagagguugcagugagcugaguccaugccauugcacucuagccugggcaacaagagugaaacucugucucaaaaaaaaaaaaaaaagaaaagaaaagaaaaauaaguguauagaacuuuauuguuucauugaccgggcacgauggcucacgccuguaacucuagcacuuugggaggccaaggugggcagauuggcugagcucagcaguuugaaaccagccugggcaacauggugaaaccuaucucuaauaaaauacaaaaaauuagcccgguaugguagcacaugccuauaguuucagcuaccugggaggcugaggcaugagaaucacuugaacccagaagguugcagugagccaagauugugccacugcacuccagccugggcaacagagcaaaacucugucuccaaaaaaagagaacuggccagggaugguggcucacgccucuaaucccagcacuuugggaggcuaaggugggcagaucacgaggucaggaguucaagaccagccuggccaacauggugaaaccccgucucuacuaaaaauacaaaaauuagcugggcaugguggcaggcaccuguaaucccagcuacucaggaggcugagacagcagaaucguuugaaucugggagguagaaguugcagugagccaagaucacgccauugcacuccagccuggguggaagggcaugacuucgucucaaaaaaaaaaaaacuuuuuccuuaaaauacaccucuuggagccagucccagauacuugggaggaucaggaaggaggaccucuugaggccauaaauuugaggauguagugugccugugaauagccacuacacucuagccugggcaacacagaaagaccucauaaauaaauaaauaggccagaugagguggcucacaccuguaaucucagcacuuugggaggccaaggcuggcagaucacgaaguuaggagaucaagaccauccuggcuaacacagugaaaccccgucucuacuaaaacuacaaaaaauuagccaggcaugguggcaugcaccuauagucccagcuacucgggaggcugaggcacgagaaucacuugaacccgggagacggagguugcagugagccgaaaucgcaccacugcacuccagccugggcaacggagugagacucugccucacacaaauaauaaauaaauaaauaaauauuuuauaauguauuuuuaaaauauaguucuuaaaacaugccaugaguagguuuaauuuuucuaaaucauagaauaggauacugaagcccagaguggucaugugucuugccuaaggucacacacagcuaauauauacaagaucaugggucuucuggcuccagucugaggcugucacaauaacuagcuucucacuugguccuaccuaagacacaggugucagcaacucuaaauucaagaaauugagauuaagacuaaaguaauagcuuauaaacauuaauuaagcauauuaauugcaagauguuaaaccagucacugagagauuaagacauugaagauacugguuucugccuuaauagagcuaacaauuuaguaaaaaagucgcaaaugagucaagacuuaaauaagacuuaaagacuuaagacaauaaaaaaggcaugauaaaaggguaaauuuaggcuggacgcaguggcucaugccuguaaucccagcacuuugggaggcugaggugggcggaucaccugaggucaggaguucaagaccagccuggccaacauggcaaagucccaucucuacuaaaaauacaaaaauuagccaggugucauggcacaagccuguaauuccagcuacucgggaggcugaggcaggagaacugcuugaacccagaagguggagguugcucaagagcaagacucugacucaaaaaaaaaaggaugggggauaaauuuagcaagaacccucaucaucccauuuaccuacagccuaauauguacuaggaacuuacauauacaauaauagguaaaaucuauuaagaauguauuauguuuuauaguugaacaaauugugucgcuuaaucaacuuagaauucauguaaaugccacauuugagcucuguuacauugagagcucuccaggcccagccuaccauguuaagccauugauaaaccucccugcuacuauuuaaaacaagauggauaccuuucuacugucuuuaaagaggaagaguacacacuguuggacuguaugcacucuccgucucucaagacaguaaucugagggauuacuucaauagcagaaguuaaaagaagcggucaguuggguuuauuacauaaucucaaucacauccugaccaauaaaaugcaccaauaaagaucagcaaauaaaagcaagugauuagaagagcaauuuuaugcucacgagcucugaauucacaagugugagauuuccugucaaguacucucaugagcucaauuaaucucugacccagggugagaaaauaaacauagugacgcagaggcugcagcucaugcuuuggaauuugucagacuugacucaguucccaacuccaucauguccugccugggugacccucauuaugcuaccuggacucucucaguuuccuucaccuguauguaacacaagagcagagccacccucaacaagggguuacuauaaaaaucgaagaacccggcaucucuaacaacaucccuggcacggagcaaucccccuucucccucagccacuggcugagcacccacagaggggaguaaguccuauugugguaaugguucucaug

SEQ ID NO: 30 - RNA, 11120 nt, Homo sapiens
ggggucccgcuauguugcccaggcuggucucaaaccccugaccuccagcaauccuccugccuuaacucccacacuauugggauuacaggugugagccacagugccuagcccucauacuuuugagaaaaaaaaauuccuaucauggggcuuuacaacuguuauuauuuuuuaauuuccaaaaauaaauaauguauuacuaguuaugguacugcucauguucacgaucuuaauucugaagagaguuaaaugcuucaagaagaguggagcucccuucuccuuagcaacaugcuuauuuucuaauagcagccuuuggcagacagugcucccaccuuuucaaugacuguugugggaauuucaaguuuggcaggaggacagaacaauauccuggcagaugaauucugcauccuucagaucguaugauguccuaaauaagacaacugacuucucuacccacucacuuucucggggacauuuacugagggccuauaaacugugccaggcauuguuuagaugcuguaaaacagaucaguaugacuuggucccuguccucaaaggaaucuaaaaacagucguaaaagacaccuauguaaacaauuaaaaugaacaggaugaaugcuauaacaaagacauuauugaaauuuuguaggaguauugaaaaagaacagguaacaagagggcucaagaaagcuucacaaagggggaacaagcuggucuuaaaagguaaagagaaguuugucaggaguagaagggauggaaaagcauuucaaauaaaaggagcagcuuguacaaagcagaaaagugugugccaaggaaugauaugaacucaugaacuucagugugaguggacuauagggaacaguguuauaaaaugaagcuggagaagaagguuggccugagauugcaaggggaccaaaaauucuguuaagaagcuaagcauuuugaccagcagcagacagccccaaacauuuucaagcagggacaucacaugagaaccaguuuuuggacaguaacaaugauuugaaacaaacgcagauaguacucagugaaucucaucuguaaguccucuacauacccugagcgguaagucauuaagaaaaagagucuagugagaaccauuuagaaucacuuucuguauggggggcuucaaaucccuccaggauauaaauaaaccucuggguuuugguuauuuuuauuugcugucuuucaacacagagcauuuauuaguuucaccaucacccuugacgacaucacuucuguuuuuuacuuaucccuugaauagauguuaucugauuguuuuaccuuucauauauaaaggauuuuugguuuuacuguugguggguguacuuuuuguaaacauaccugugaugcugcuccaacaucccugcuaagagcuuugcugauuuuaacaaguaugugauuccacugcuguaagccaaggguaaacuugauuccaccaaauuugcuuaaguguuacuucucuguauuaaagcuucaaguucacucacugucuaaccaaugauacacagcuaccugaagacauuaaaaucuuucagaaauuuauucucaauuuuuuucuucuuauaaauuacuagcauaucaggauaaagaauaaagugugugaagaggcaaaauaaguguguaccacuaacuugaaaauuccgcaacaggugucaccuuaguuuuaacacauucuucuguucaaagauauaaauucagaaucuuugggaguaguuaauuaggugcaucugugagaugaacaccuauuacagaguuggacacaggaguagcauucaaacaauacccuuuaauagauacaaaaauagaaaaacacaaaagagaauacauggacacaugagacuaauuuuuagauaaaagcaauuuguagauaguucuuuaauuugacuaaaaggaaagugaagaugucuaaauauccauaccugucauaugcuuuauacuauauaauauaauauauugguucaauuuucuuuuauaaaaucauaucauucuaauaaacaaaccuucacauaauauaaaaguaggaaaacuuggacuucugaaaccugcaaugugucugaaauacauaaacuagaauagcagcaaaagauagaaauuaagccuuccaugauuuugaagucugccuaucaucaaugcucuuaauuaacuuuugaaguugugauuucuuuugaagaaaaaaaaaaacuaaagugcaggagucuucuucaauacugaguuuuaguaaacauaaguuucuuuaauuaucugguuuaauucaauaaaauaauuuuauuguguaauuuacuucucuaaaggugaaauggaaaacaacuuauccaaacagcauuauaagagaaggacuaccggucugacaaaauucugccucuaaggaacguggugaauauucugggauuaugaaagccaacuuggcauauuacgaacacugacuuuuauaaauaacagaucuuuauaagugcuaauaguacacguuuaauuugguaaacaacaacaacaacaggcauuuccagcauuaaacagaauuaaacaauuuggaugugaaaauacauaggaacuauguuucuuggcucaauaauuuuuaaguacuagucucacaaggaacugaaguguaguaauuacuuauguucaacagauggcgaccuuuacauuguagucagcaaaacaguauuugcauaaaacaguauacucaaccagcagaaaaucaaagaucaauuacaaauauuccauauuuugcaagagacaaaaauuacuugaucauauacaccauuuuuugacaggugguuuuuuuacagguuggcaaacauauuuugugggaauguaucuugcuguucuauuuuuuggggcaaguccaauugcuaauucuuaucucaugaaaauaugugcaaauaucaggcuagggaauaauuccugcuuucuuugcauagcuuauuaagaauagcagcuuuagguaauauacucaacuaacgugucuuaauaaggauaugucacaagacaccaguauguuugugaaacacagcaauaaugaagacaugggaugggucagcaaacuauuucuucauuaaucuauuuugugugggucaaugaauaucaacaaaguuuuaaaaaaacuuacaaaacaguauguucagaaauauguaauuuaugcuuccuaaauuuucaaugaauguacaaagguaauagguuucuccuuaagaaguaacauuaacucuauucuuccagugcuaaaauuccauucugauggccuucagccagaaaucaugugggcauuauuuggugcuagaagauagccuacaagaagacgguuucccggcucaacauaacuguuuuaguuuguguauuguaauugguaaccugaguuugaauuguuuuucuacuaaagggagaagcagucacuccucuccuaaugagcauuuuaauucuuugacaauugggaagccauaucauaccuuauaauucuuaaccauugauaugguuuggcucuguguccccacccaaaccacaugucaaauuguaauccccacguguugagggagggaccuggcaggagguaccuggaucauuggggugguuuucccaugcuguucuugugacagugagauuguucucaugagaucugguuguuuaaaaguguguggcaccucccccuucucucucucuccugcuccaccauugugaagauguaccagcuuccccuucaccuuccaccaugauuguaaguuuccugaggacucccacccaugauuccuguuaagccuguggaacuaugagucaaucaaaccgcuuccucauaaauuacugucucagguaguucuuuuuagcaguaugagaacagacuaauacaaccauaauaaauaugugaaaaaucagaacccaucauucuuuaagcaagggagauaaagaaaagacugauaaaaauguuuugaucauuauuuuagauauccuuuugcuaaacuaaaauaccacuuuauacuuauauauagcauuaaaaauuugaaacuacaaagauugauauuauuauaauauaguagaaaauugagaacauggugagaucaagaaaaacauuuuuuaacuuaaauauacuuugacaauaugauuuuuuggcauaaaugugugugugugugugcauguauaacauaauuaucucuaaauaaaauuuucuauggucucccauaauucucuauucugguaggccauauauuauuaauaacuuucuauuauuaauaguuacugauaagauggcuguugccucuuuuaaaucaauguuguagauuagauucuuauauuuaaacauuuccuuauuaaugauaaaauuuucucuauuaguauuucuguucagagcacuaacuaagcaucucauauaccuauuucuaaaauuguuauucucuccucaugggaaucaccaugcucuucauguugaaacacaccaaaaacacaaaggaagacauuaaaaaacaaauguucauuuaaugaaaaaaagucuuucacuaauuaacaaacagaaaauaucaagaaaaaagugucaacuuuuggcaauuagauuacuccaacaaaguacaauuccccuuucaagugacuuagguaugguuucacuuuacuuucaauauagguauuaauguuuugauauuuuaaugucuugauauuuauaucgaacuuauauuccauuagagauagugugugccacgcuguaaacaacuucccuaaaauuagagaauuggaaaaauaugcaauuauauauaauuaguaacuuuuaguaccaaacaugguggggcauacuaaaauucucacauucaaagaacuuuuacuguaguugagaaaagagaauauggagacagauaccugucauccaaagguaaaaaggaagcaccaaaagagaggugacaacaaugcuuccaauuggguccaagucaaugauucuacuuaagaauaauuuuuucuuaaaugacaaucugucaaaaugaggauuaguauacaaagacaguuaguguauguuauggaccgaacuguaucccuccauuauucauauguugaagcccuaacccacaaugugacuauuuggagauagguccuuuaaagagguaauuaagguuaaaugaagucauaagggugggacccugacccaauagaacugauguccucaugagaagaagagaccccagagcuuucaugagaagaacagaccccagagcuuucacucuuucucugugcccccacagagaaaaagccaugugaggagacacagugagacaguggccauacacaaccguuaggaaaggccucaacagacaccaacucugcuggcaccuugaccuagaacuuacagucuccagaacuaugagauaauaaguuucuacugucccaccuaaccagucuguguaucuuguggcagccuaaacugacugaaaacaguauauuucuaacugaagaaacaguucaccagagaugaauuuuauaaucuuaacaaccaccuucaagauuagucauuucauugaauugcuaggccuuuacuuacaguugcagcuacaguugcauccaggaagaaauacgaugacuacuagaaugccuuuuccuuccaauuuauacuauuacacuaagacuuuuguuuuuaaagagcucaaacuucgguaggaaacuuaagacauguagaccaauauccauaagacaaauuagaaaaucaagugucaaaugagagguaaaaauuguguagcauguaaguucagaggugacaaaauuauuccuacugugggguggggcaguggagagggcgcucuguaggugagaauguaugauugugggaaggaauaagaaaaucuaggccagacgcaauggcucacaccgacgauccuaggacuucgggaggcugaggcaggcagauugccaagaguucgagaccagccugggcaacauggcaaaauccugccuuuacaaaaaauuagccaggugugguggugugugccuguaguuccagcuacuugggaggcugaggcgggaggaucacuugagccugggaggucgaggugcagugagccaugauggcgccacugcacucuagccugggugacaaagcgagacucugucucaaaaaacaaaacaaaacaaaacaaaacuuggaaaagggaauauuuacacucaaugcagucauucucaguagcacacaauuggcauauugggcuggaugauucuucagucugugggauucuucagugcauuacaagauauuuagcauccuccuuccuuuuaaugucuguacauuagccuacacauuuccaaaggcuacucagcaacagaaucuccccaggagagaacuagaaguaaacuuuuaaggaugugaagaaaacagauguguucuaacaaaauuuauuaagugaaauuaaauggcuaauacuuaagauuccaaaugcuauauauggaagacuauaacaugcaaaaucauaauauuuuaaguacugcuuuccuguuuuaaguguucauuacauuuacucaguaaauccuaauuucccauuuugaagcuucucauuucauugcuucauucacauaucucugauuauauuugauuucuaaauacauuugcuugcucauuacucuuaggcugccugugaugaagucaacaaauaaacauccuuaauuuaaggcaaaaaaauaaaguaaaugcauguuccuuuugacuuccacaugacuuucaguccucccuuaacugaucuuuuacuuugaagacuucuaaaugcuguguuugugaucuggguugaagaucuaaggagaaaagaugagaagugaccaaggcaaagaaagugaugagaagaugaauaaaagagaaccugugcaacugaggucuaaacuggggucuacaaaaauaugauucuuuugcagaucagguucuccagaaagcagagcuuagcgcuuagcaugcaggaugcuuauuaagaaaugucucuagggucaacaaccgugaaggggaaaggacagaaaaggaagcaggaguggguaggagagguggaguugcaaacaaaacuuaggacagccucagcugauaccacagcagcucaggagcuagaauggucccuccaaguuggucuuaauugggaugaaauaaccagguuuuuuacuucccucucuucccacuuaucaauuaguucacugcaugugggcagcaccaccaagccuagggucuugaagagggacugucugcagcugaggugauuugugaaggggcuggcacgugaaggcugucugccuacgacacucccaucagcagagguaucaaguccuccacugaagugguaucugagugggcagcacaguguccaccacagauuccaacuccauauuuuaaagaaguggaaacuuggggacagaaaguaaucagugaguuacaaaucucagucuaaaccccagaauuuaucacuugcugcuucuuacuacuucuaaccaagaaaaaaaugcuaugaucccuauuuuggaaagaaugugcaaggaaauuauuuacuugguuuaaggaauuuucuggaaugggaugaacaaaagcaaaguguccagacaauucuagcuaagauuuuacagagaagacuaaauccucaaauucaucacuauugugaagagcaucauaugucaaauguaauacaccaaaguguuuucguauuuucuguauggcuuuauguaugcucugcaggauauucuguuuguguucccuacauucauguuaccaacauauauuuaugccucucuuuucuaggucuucauugggucucaacaggaaaaauauuaaauuguuaauaaucagggcugguaccaacuagaugagcacacugcaauuagcagccugacuaaccucccugcuucuucauuuucuaauuuuauuugcaagcaguuagauaugauauaauaauuucccauuuaaaaugcuauggauaggaaugggaauauaaguguuuugucuauguacaggaguucuguuuuucuagcacacuucccugccuagccuggacuccagccacacugaccuuucuguucuguauucuugccaugcuccuuuccacacuugucuccccacccucaagucaucacacggcugacuccuuuuccacucccagcugcuggcauuaaggucgucuccucagagguccacauaaucuaaaguaauccuucccugacuaagccaaacuuauuuuauauuaacuuauuaucuugguaacacaauuaccucagaucaucuugcuuauacauuuaucuguuauucauuacuguaucccuaguacauugauuagugccuggcauuuacauauacagcaauuaugcauuugaaagaaugaaugcuggcaaugaguaaauauuuacaauuuauaugauuuuaaauacacaucagaaacacaauaauuacaaaauuacauaucuggagggaaaaucaauucauucuauauuuuugaauaauuuugaaaaaaagaaggaacugcuacucuguguccuaauaccacaauuuguauuaaauaaacucuaagaugucuaucuaucugagccuuccuaaagggcacaaugaugcacgaaagaaugcuuuuguuuuccacaaauacuguaauuuuugagguaacaaaaauacauuggaccaugugugagacagucuucugaauaaaacacuguaagaaaaaaauguugaggccaggcaugguggcucacaucuguaaucccagcacuuugggaggccgaggcacaggcggauaacuggaggucaggaguucgagaccagccuggccaacauggugaaacccugucucuacuaaaaaaacuauaaaaauuagcugggcagguggugcacaccuguaaucccagcuacucaggaggcugaggcaggagaaucacucaaaccugggagguggagguugcaguaagccaauaccgcaccaucgcacuccagccugggugacagagcacaacucugucucaaauuuaaaaaaaggaaagaaagaaggaaaaggaaagaaaagaaaagaaaagaaaagaaagaaagaaagagaaagagagaaagaaagguugaaaacauucuuaagagcaacuucccaauuucacuuuaaaaacaagccuauaaugaagaaagucuguauaagagagacaaacaaaaacaagucauaaauuaggucacuagauuuacaauaauauuuuugcaagaagcuuuuauaauuuuuggcuagcauuuaagcuaggaauauaaaaugucccaggagauucacugaacucaggaauucaucuucugacagggucaaauggaauguccuuuaugauccuggcugaauauuuauccucacacucaaguauggaucauuccauuuaucuaagucuaugcuuuuaaugaaacacuaaauacuucauuuuuaauuaagcaagguaauaaauuuuuauuuuaacuacaucuguuuuaaguuguaacucuauacauguucucuugucuguauuagggaaaauuuuucccauuuaccuaaaauucuuuugagucuucuuucugucaacucuguaguuauucuagaaauuggauucuugguggaaaauaccauguuuacucuaccacaccauuuauuuuuaugaggcuuuaauuccaucgucucucagacaucaucuuuuaggauccuggaagaauuacuggguauaggaaagccuggucuaagaaaguagauuuucuuacagaggccagagacacaguuauaacugggauugaugucagcaucauaaaaacacaagcaggcaggaccucugucuuaguaucugaagaacugauaugacaaaagaaagaugucuuauauaucaugagguauacuagaaaugagcauagauaaauccuagaauuauuuuuaaaaugcauuuggcaggccaaauuuuaucaaucacuaucuaaaauuaauguuaguuucuccaaauuaucaagcaguuguacuagacuauaaugucacuaaugugaaagaauaccuggaaaaauaaaaauagacccuugaauguaggcaagguccuucagcgguaauuucacacaaacaauaauucuauguugcacaauagucaacugauuuuauguuacucacauaggauacuacauauuguaauaauucagggggaaaaucaaacaugauccuuuacugugucuuauuuauuuauuuauuuauuuauuuauuuauuuauuuauuuagagauggaguuucgcucuuguugcccaggcuggaaugcaauggugcaaucucggcucaccgcaaccuucgccucccggguucaagcgauucuccugccucagccucccgaguaccugggauuacaggcaugcgccaccaugcccggcuaauuuuguauguuuaguagagaugggguuucuccauguugguuagucuggucuugaacucccaaccucaggugaucugcccgccucggccucccaaaaugcugggauuacaggcgugagccaccgcaccuggccugccguguuuuauacccauauuuuaguaaccucacaaaguuaaaaaugauuuccaaaugaaaugucagagaauuguuuauuuuacccaguuauacuguaauaaguccuggauggcacugugucuagccucaaggaaacagaacaaguacaaagcuaaguuaaaggcaacuuuauuacaauacuuuuccaaacuucaaaaugcauuuauuacuaaauuuagcucuaauauauaauacacuauauuacaaggcagaaagcauuucuggucauauauuuagcuuccauguauugcccaaaacacagcuuccuaggaauagcagcagugcacaguaguuuacgucacuaaguaaaauauuucucugaugucucacaaucauucauauuguaauuuauuuuauguucuucugauuaaaaaaagcaaagcuaucuuuuccauaucuuauuaguuaauaccuagaaauugucuauauucuaaaaauauccacuagagauauuuccuuugauaggaaauaggauuugcaaauccaaccaaaccaaagaauacuguauuucuccauugcaaauagggguaaaauucucuucuuuccuuuuucaggagccaaaucuggaccuccuccucuucauagagccauccugaauaguaccuacuuuaaaagauaccuaaaucu

(19) United States
(12) Patent Application Publication
(10) Pub. No.: US 2018/0363065 A1
(43) Pub. Date: Dec. 20, 2018

(54) USE OF ANTISENSE LONG NON-CODING RNAS FOR THE DIAGNOSIS OF PROSTATE CANCER

(71) Applicants: INSTITUT CURIE, PARIS CEDEX 05 (FR); CENTRE NATIONAL DE LA RECHERCHE SCIENTIFIQUE, PARIS (FR); SORBONNE UNIVERSITE, PARIS (FR); INSTITUT NATIONAL DE LA SANTE ET DE LA RECHERCHE MEDICALE, PARIS (FR); ASSISTANCE PUBLIQUE - HÔPITAUX DE PARIS, PARIS (FR); UNIVERSITE PARIS 12 - VAL DE MARNE, CRETEIL (FR)

(72) Inventors: ANTONIN MORILLON, PARIS (FR); ARTURO LONDONO-VALLEJO, PARIS (FR); YVES ALLORY, PARIS (FR); ZOHRA SACI, MONTREAL (CA)

(21) Appl. No.: 16/062,639

(22) PCT Filed: Dec. 16, 2016
(86) PCT No.: PCT/EP2016/081348

§ 371 Date: Jun. 15, 2018

(30) Foreign Application Priority Data
Dec. 17, 2015 (EP) 15307036.2

(51) Int. Cl. C12Q 1/6886

(52) U.S. Cl. CPC C12Q 1/6886; C12Q 2600/16; C12Q 2600/178

(57) ABSTRACT

The present invention provides three new antisense long non coding RNAs (lncRNAs) that allow to diagnose prostate cancer much more accurately than the existent non-invasive diagnostic tools. So, this invention relates to the use of at least one of these antisense 1ncRNA or a combination thereof as a diagnosis marker for prostate cancer. It also relates to an in vitro method for prostate cancer diagnosis of a subject as well as to a kit for performing this method.

### FIELD OF THE INVENTION

The present invention relates to the field of medicine, in particular of oncology. It provides new diagnostic markers in prostate cancer.

### BACKGROUND OF THE INVENTION

Over the decade, cancer of the prostate has become the most commonly diagnosed malignancy among men and the second leading cause of male cancer deaths in the western population, following lung cancer.

Early detection and treatment of prostate cancer before it has spread from the prostate gland reduces the mortality of the disease. This realization has prompted increasing efforts for early diagnosis and treatment. Indeed, the American Cancer Society recommend that male population at large undergo annual screening for prostate cancer beginning at age 50. The recommended age for screening is lowered to 40 for men giving a family history of prostate cancer or other risk factors.

Early screening and detection could also translate into a reduction in the healthcare burden, as early treatment can be less radical, more successful and therefore provided at a lower cost per treated patient. The key to accomplish this goal is to provide better differential diagnostic tools.

Nowadays, screening for prostate cancer involves mainly palpation of the prostate by digital rectal examination and assay of plasma levels of prostate specific antigen (PSA). PSA is a serine protease produced by the prostatic epithelium that is normally secreted in the seminal fluid to liquefy it. Disruption of the anatomy integrity of the prostate gland can compromise the cellular barriers that normally restrict PSA to within the duct system of the prostate, allowing it to disperse into blood or urine. A number of conditions can result in this leakage of PSA, including inflammation of the prostate, urinary retention, prostatic infection, benign prostatic hyperplasia, and prostate cancer. It is therefore not surprising that screening of serum PSA as an indicator of prostate cancer is not absolutely predictive.

This low level of specificity results in additional more invasive and costly diagnostic procedures. Indeed, the normal procedure for a subject positive to PSA and palpation is to proceed to a biopsy which consists in 10 to 15 extractions of prostatic tissue. Among the subjects tested, only 45% appear to really have a cancer and 10% develop a prostate infection after the biopsy. Biopsies, when unnecessary, are also very traumatic for the patients. The psychological impact of being diagnosed as positive until proven as a false positive should not be understated either. Moreover, even a biopsy is not always 100% certain, and a second biopsy procedure is often required.

High-throughput RNA sequencing has produced catalogues of long non-coding RNAs (lncRNAs) with now 58648 lncRNAs annotated (Iyer M K et al, Nat Genet, 2015, 47, pp. 199-208). LncRNA are RNA of at least 200 nucleotides long, cell type/tissue specific and poorly conserved during evolution. Among different classes of lncRNAs, natural antisense transcripts are the less described with only 4200 annotated antisense lncRNAs (Derrien T et al, Genome Res, 2012, 22, pp. 1775-1789), certainly due to their low expression. In addition, most genome-wide studies were focused on lncRNAs without addressing their strand-specificity explaining a lack of systematic characterization of antisense transcriptome.

LncRNAs have been shown to exhibit oncogenic or tumor suppressive functions in cancer biology. Being highly tissue specific and deregulated during tumorigenesis, an increasing number of lncRNAs were also proposed as biomarkers for diagnostic, prognostic and/or monitoring purposes. So far, only one lncRNA, PCA3, received the FDA approval for decisions to perform a biopsy in a prostate cancer (Groskopf J et al, Clinical chemistry, 2006, 52, pp. 1089-1095). Although addition of the PCA3 score globally improves diagnostic accuracy, it remains inaccurate in some tumours, especially among old patients and patients with advanced cancer, especially cancer relapse.

In view of the fact that advanced prostate cancer remains a life threatening disease reaching a very significant proportion of the male population and the number of unnecessary biopsies, there is a strong need to provide the most specific, selective, and rapid prostate cancer detection methods and kits. Thus, further investigations are still needed to identify new biomarkers and to evaluate their clinical performances. The present invention seeks to meet these and other needs.

### SUMMARY OF THE INVENTION

The inventors have discovered three antisense long non coding RNAs that present more robustness and better diagnostic precision of prostate cancer than the actual diagnostic PCA3 marker.

Accordingly, in a first aspect, the present invention concerns the use of at least one antisense long non-coding RNA (antisense lncRNA) or a fragment thereof of at least 50 nucleotides as a diagnosis marker for prostate cancer, wherein said at least one antisense lncRNA is selected from the group consisting of asFBXL7, asABCC4 and asPDLIM5.

Preferably, at least three antisense lncRNAs are used in combination and said combination comprises asFBXL7, asABCC4 and asPDLIM5.

The present invention also concerns, in a second aspect, an in vitro method for prostate cancer diagnosis of a subject, wherein the method comprises the step of determining the amount of at least one antisense lncRNA or a fragment thereof of at least 50 nucleotides in a biological sample from said subject, said at least one antisense lncRNA being selected from the group consisting of asFBXL7, asABCC4 and asPDLIM5, and wherein an increased amount of the at least one antisense lncRNA is indicative of prostate cancer.

Preferably, the amounts of at least three antisense lncRNAs are determined and said at least three antisense lncRNAs comprise asFBXL7, asABCC4 and asPDLIM5.

Optionally, the amounts of additional prostate cancer markers or fragments thereof are determined, preferably antisense lncRNAs prostate cancer markers or fragments thereof, more preferably the PCA3 antisense lncRNA or a fragment thereof.

Preferably, the sample is a body fluid, more preferably a urine sample.

Preferably, the antisense lncRNA amount is determined by amplification or by hybridization, more preferably by quantitative RT-PCR or by the Nanostring method, even more preferably by the Nanostring method.

The subject is a mammal, preferably a human. In particular, the subject is a man, preferably an adult man, more preferably a man of at least 50 years old.

The present invention also concerns, in a third aspect, a kit for the diagnosis of prostate cancer in a subject, wherein the kit comprises (i) probes and/or primers capable to specifically hybridize to asFBXL7; and/or (ii) probes and/or primers capable to specifically hybridize to asABCC4; and/or (iii) probes and/or primers capable to specifically hybridize to asPDLIM5; and, optionally, a leaflet providing guidelines to use such a kit.

Optionally, the kit further comprises probes and/or primers for the detection of additional prostate cancer markers, preferably probes and/or primers for the detection of antisense lncRNAs prostate cancer markers, more preferably probes and/or primers for the detection of the PCA3 antisense lncRNA.

Preferably, for each antisense lncRNA, the kit comprises two probes:
A capture-probe which comprises a nucleotide sequence hybridizing a first part of the antisense lncRNA and a molecule able to bind a solid support; and
A reporter-probe which comprises a nucleotide sequence hybridizing a second part of the antisense lncRNA and a detectable label.

The present invention also concerns, in a fourth aspect, the use of a kit as described above in the diagnosis of prostate cancer in a subject.

Preferably, the subject is an animal, more preferably a mammal, even more preferably a human.

In a most preferred embodiment, the subject is an adult man of at least 50 years old.

### BRIEF DESCRIPTION OF THE DRAWINGS

FIG. 1: Novel antisense lncRNAs are overexpressed in prostate cancer. PCA3, a known antisense lncRNA, and three new antisense lncRNAs (asFBXL7, asABCC4 and asPDLIM5) are highly expressed in a set of tumors (16 samples) compared to normal tissues (8 samples), measured by RNA-seq.

Boxplots of the expression of the three new antisense lncRNAs are compared to PCA3. Normal tissues are referenced with the suffix “nor” (e.g. PCA3_nor), prostate cancer tissues are referenced with the suffix “Tu” (e.g. PCA3_Tu). The mean is indicated by a horizontal bar in each box plot. Dispersion of expression is represented by vertical dash lines. The three novel antisense lncRNA have much less dispersion than PCA3.

FIG. 2: Antisense lncRNAs are a statistically significant novel signature for detection of prostate tumors. (A) The 3 antisense lncRNA are highly expressed in tumors samples from patients with or without relapse. Nanostring measured RNA levels in 89 cohort of patients using 3 probes against asFBXL7, asABCC4 and asPDLIM5 normalized on references mRNA expression. Signals are expressed in relative expression scale for each patient (triangle). The mean is indicated by a horizontal bar in each box plot. Normal tissues are from healthy patient (first box) or from healthy region of prostate with cancer. No and yes represent patient without or with relapse after 10 years. (B) PCA3 is highly expressed in tumor samples but with less dispersion and higher levels in non-relapse patients. Same as FIG. 2a but with PCA3 dedicated probes. (C) ROC curve for detection of relapse prostate tumors within a cohort of 89 patients using PCGEM3 lncRNA, PCA3 alone, sum of novel 3 antisense lncRNAs (asFBXL7/asABCC4/asPDIML5) or total of all the 4 transcripts (without PCGEM3) measured by NanoString. AUC (Area Under the Curve) values indicate the specificity of each set to detect high-risk tumors. Value higher than 0.90 are statistically relevant.

FIG. 3: The 3 antisense lncRNA are detectable in urines of cancer patients. Nanostring measured RNA levels in samples extracted from prostate biopsies tissues (198KU, 156MAR, 065GUI and 076GBE), commercial samples mixing different cancers (Ref cancer, a mix of Adenocarcinoma, mammary gland, Hepatoblastoma, liver Adenocarcinoma, cervix Embryonal carcinoma, testis Gliobastoma, brain Melanoma Liposarcoma, Histiocytic lymphoma, macrophage, histocyte, Lymphoblastic leukemia, T lymphoblast, Plasmacytoma myeloma, B lymphocyte),normal prostate RNAs (Ref ProstN), urines of 2 prostate cancer patients (001ANO and 003ANO), or breast cancer cell lines or tissues (PAMS38, MDA MB361, SKBR3 and HMEC) patients using a PCA3 probe (first bar of each sample results), a PCGEM1 probe (second bar of each sample results), or 3 probes against asFBXL7, asABCC4 and asPDLIM5 (third bar of each sample results). Results are normalized on references mRNA expression.

FIG. 4: Prediction of high sensitivity and specificity of the 3 antisense lncRNA. ROC curves for prediction of sensitivity and specificity of prostate cancer diagnosis within a cohort of 186 patients using as FBXL7 lncRNA, asABCC4 lncRNA, asPDIML5 lncRNA, and a sum of the three antisense lncRNAs (asFBXL7/asABCC4/asPDIML5) measured by NanoString. AUC (Area Under the Curve) values indicate the specificity and sensitivity of prostate cancer diagnosis of each set. Value higher than 0.85 are statistically relevant.

### DETAILED DESCRIPTION OF THE INVENTION

Recent data have uncovered a great number of long non-coding RNAs (lncRNAs). However, until now, antisense lncRNAs have been poorly studied despite their importance in disease and tissue specificity. By performing RNA-sequencing analyses, the inventors have discovered three novel antisense transcripts that are highly specific of prostate cancers and can be used as diagnostic tools for cancerous lesions. These new antisense lncRNAs appear to present more robustness and better diagnostic precision than the actual diagnostic PCA3 marker, recently approved by FDA.

The actual diagnosis procedure, i.e. prostate palpation and PSA assay followed by biopsy, leads to 55% of misadapted biopsies (the subjects tested doesn't have a cancer). With the inclusion of the test of these three new antisense lncRNA in the procedure, the percentages of misadapted biopsy fall under 10%. This new diagnosis test is highly specific (more than 90% of the biopsies are positives), rapid (results are obtained the day of the urine collection), and cost effective (unnecessary biopsies are avoided).

### Definitions

As used herein, the term “antisense lncRNAs” refers to a subset of long non-coding RNAs being natural antisense transcripts.

Long non-coding RNAs are non-protein coding, actively transcribed single-stranded RNAs. Mainly nuclear, lncRNA transcripts are subjected to processing in a manner analogous to mRNAs: they are capped, polyadenylated and frequently spliced. They are also cell type/tissue specific and poorly conserved during evolution. Their length, more than 200 nucleotides, distinguishes lncRNAs from small regulatory RNAs such as microRNAs (miRNAs), short interfering RNAs (siRNAs), Piwi-interacting RNAs (piRNAs), small nucleolar RNAs (snoRNAs), and other short RNAs.

As used herein, the terms “antisense lncRNA”, “(as)lncRNA”, and “Natural Antisense Transcripts (NATs)” are used interchangeably and have the same meaning. An antisense lncRNA is a lncRNA that is a natural antisense transcript of a given gene, which means that the sequence of an antisense lncRNA is complementary to the pre-mRNA sequence of said given gene. Therefore, the sequence of the antisense lncRNA cannot be complementary to artificial sequences, such as morpholinos, that are antisense to said given gene.

The term “pre-antisense lncRNA”, as used herein, refers to the nascent antisense lncRNA, prior to its maturation (capping, polyadenylation and splicing).

As used herein, the term “splicing” refers to a modification of a pre-RNA transcript in which introns are removed and exons are joined. “Alternative splicing” refers to a particular splicing process that can create a range of unique RNA splicing products from the same pre-RNA. Alternative splicing can occur in many ways, exons can be extended or skipped, or introns can be retained.

The term “cancer” or “tumor”, as used herein, refers to the presence of cells possessing characteristics typical of cancer-causing cells, such as uncontrolled proliferation, and/or immortality, and/or metastatic potential, and/or rapid growth and/or proliferation rate, and/or certain characteristic morphological features. This term refers to any type of malignancy (primary or metastases) in any type of subject. In particular, the term encompasses prostate cancer at any stage of progression.

As used herein, the term “diagnosis” refers to the determination as to whether a subject is likely to be affected of a prostate cancer. The skilled artisan often makes a diagnosis on the basis of one or more diagnosis markers, the presence, absence, or amount of which is indicative of the presence or absence of the prostate cancer. By “diagnosis” is also intended to refer to the providing of information useful for diagnosis.

As used herein, the term “diagnosis markers” and “biomarkers” are interchangeable and refer to biological parameters that aid the diagnosis of prostate cancer. It is a measurable indicator of the presence of this disease. This term refers particularly to “tumor diagnosis markers”. Tumor diagnosis markers are substances that are produced by cancer or by other cells of the body in response to cancer. Most tumor diagnosis markers are produced by normal cells in the absence of cancer as well; however, they are produced at much higher levels in cancerous conditions. These substances can be found in the blood, urine, stool, tumor tissue, or other tissues or bodily fluids of some patients with cancer.

As used herein, the term “treatment”, “treat” or “treating” refers to any act intended to ameliorate the health status of patients such as therapy, prevention, prophylaxis and retardation of the disease.

As used herein, the term “effective amount” refers to a quantity of a pharmaceutical composition which prevents, removes or reduces the deleterious effects of the prostate cancer.

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof). Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “sequence identity” or “identity” refers to an exact nucleotide to nucleotide correspondence of two polynucleotides. Percent of identity can be determined by a direct comparison of the sequence information between two molecules by aligning the sequences, counting the exact number of matches between the two aligned sequences, dividing by the length of the shorter sequence, and multiplying the result by 100.

As used herein, the term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

The term “hybridization”, as used herein, refers to “nucleic acid hybridization”. Nucleic acid hybridization depends on a principle that two single-stranded nucleic acid molecules that have complementary base sequences will form a thermodynamically favored double-stranded structure if they are mixed under the proper conditions. The double-stranded structure will be formed between two complementary single-stranded nucleic acids even if one is immobilized.

As used herein, the term “amplification” refers to the amplification of a sequence of a nucleic acid. It's a method for generating large amounts of a target sequence. In general, one or more amplification primers are annealed to a nucleic acid sequence. Using appropriate enzymes, sequences found adjacent to, or in between the primers are amplified.

The term “probe”, as used herein, means a strand of DNA or RNA of variable length (about 20-1000 bases long) which can be labelled. The probe is used in DNA or RNA samples to detect the presence of nucleotide sequences (the DNA or RNA target) that are complementary to the sequence in the probe.

The term “primer”, as used herein, means a strand of short DNA sequence that serves as a starting point for DNA synthesis. The polymerase starts polymerization at the 3′-end of the primer, creating a complementary sequence to the opposite strand. “PCR primers” are chemically synthesized oligonucleotides, with a length between 10 and 30 bases long, preferably about 20 bases long.

As used herein, the term “complementary DNA” (cDNA) refers to recombinant nucleic acid molecules synthetized by reverse transcription of a RNA molecule, for example an antisense lncRNA.

As used herein, the term “hybridizing conditions” is intended to mean those conditions of time, temperature, and pH, and the necessary amounts and concentrations of reactants and reagents, sufficient to allow at least a portion of complementary sequences to anneal with each other. As it is well known in the art, the time, temperature, and pH conditions required to accomplish hybridization depend on the size of the oligonucleotide probe or primer to be hybridized, the degree of complementarity between the oligonucleotide probe or primer and the target, the nucleotide type (e.g., RNA, or DNA) of the oligonucleotide probe or primer and the target, and the presence of other materials in the hybridization reaction mixture. The actual conditions necessary for each hybridization step are well known in the art or can be determined without undue experimentation. General parameters for specific (i.e., stringent) hybridization conditions for nucleic acids are described in Sambrook, et al., Molecular Cloning: A Laboratory Manual (3rd Edition, 2001). One of skills in the art will in particular appreciate that as the oligonucleotides become shorter, it may become necessary to adjust their length to achieve a relatively uniform melting temperature for satisfactory hybridization results.

The terms “quantity,” “amount,” and “level” are used interchangeably herein and may refer to an absolute quantification of a molecule in a sample, or to a relative quantification of a molecule in a sample, i.e., relative to another value such as relative to a reference value as taught herein, or to a range of values for the biomarker. These values or ranges can be obtained from a single patient or from a group of patients.

In a first aspect, the invention relates to the use of at least one antisense lncRNA or a fragment thereof as a diagnosis marker for prostate cancer, wherein said at least one antisense lncRNA is selected from the group consisting of asFBXL7, asABCC4 and asPDLIM5, and wherein said fragment has a length of at least 50 nucleotides.

In a second aspect, the present invention also concerns an in-vitro method for prostate cancer diagnosis of a subject, wherein the method comprises the step of determining the amount of at least one antisense lncRNA or a fragment thereof in a biological sample from said subject, said at least one antisense lncRNA being selected from the group consisting of asFBXL7, asABCC4 and asPDLIM5, wherein said fragment has a length of at least 50 nucleotides, and wherein an increased amount of the at least one antisense lncRNA is indicative of prostate cancer.

Antisense lncRNA

As used herein, the term “asFBXL7” encompasses the pre-antisense lncRNA having the nucleotide sequence of SEQ ID No.1, the different antisense lncRNAs obtainable by splicing and alternative splicing of the pre-antisense lncRNA and the RNAs having a nucleotide sequence identity to the pre-antisense lncRNA or to any of its splicing products of at least 90%, preferably of at least 95%, more preferably of at least 99%. The antisense lncRNA asFBXL7 is transcribed from a non-coding and non-annotated gene present on chromosome 5 from coordinate 15902312 to 15824308. Its sequence is complementary to the pre-mRNA of the FBXL7 (F-box and leucine-rich repeat protein 7) gene (Gene ID: 23194) but shorter than the sense gene.

As used herein, the term “asABCC4” encompasses the pre-antisense lncRNA having the nucleotide sequence of SEQ ID No.2, the different antisense lncRNAs obtainable by splicing or alternative splicing of the pre-antisense lncRNA and the RNAs having a nucleotide sequence identity to the pre-antisense lncRNA or to any of its splicing products of at least 90%, preferably of at least 95%, more preferably of at least 99%. In particular, two splicing isoforms are disclosed in SEQ ID No.28 and SEQ ID No.29. The antisense lncRNA asABCC4 is transcribed from a non-coding and non-annotated gene present on chromosome 13 from coordinate 95925901 to 95948506. Its sequence is complementary but shorter to the pre-mRNA of the ABCC4 (ATP-binding cassette, sub-family C (CFTR/MRP), member 4) gene (Gene ID: 10257).

As used herein, the term “asPDLIM5” encompasses the pre-antisense lncRNA having the nucleotide sequence of SEQ ID No.3, the different antisense lncRNAs obtainable by splicing or alternative splicing of the pre-antisense lncRNA and the RNAs having a nucleotide sequence identity to the pre-antisense lncRNA or to any of its splicing products of at least 90%, preferably of at least 95%, more preferably of at least 99%. In particular, a splicing isoform disclosed in SEQ ID No.30. The antisense lncRNA asPDLIM5 is transcribed from a non-coding and non-annotated gene present on chromosome 4 from coordinate 95545972 to 95507306. Its sequence is complementary but shorter to the pre-mRNA of the PDLIMS (PDZ and LIM domain 5) gene (Gene ID: 10611).

In a preferred embodiment, the above mentioned use and method relate to a combination of at least two antisense lncRNA, said combination comprising two RNAs selected from the group consisting of asFBXL7, asABCC4 and asPDLIM5. For instance, the combination can be selected from the group consisting of asFBXL7 and asABCC4, asFBXL7 and asPDLIM5, and asABCC4 and asPDLIM5.

In a more preferred embodiment, the above mentioned use and method relate to a combination of at least three antisense lncRNA, said combination comprising asFBXL7, asABCC4 and asPDLIM5.

The above mentioned combination may further comprise additional antisense lncRNA, preferably antisense lncRNA which are diagnosis marker for prostate cancer, more preferably PCA3 (Gene ID: 50652).

In a particular embodiment, the above mentioned use or method relate to a combination of no more than ten antisense lncRNAs or fragments thereof, preferably no more than 5 lncRNA.

The above mentioned use and method refer to an antisense lncRNA or a fragment thereof. As used herein, the term “fragment” refers to a portion of an antisense lncRNA constituted of consecutive nucleotides. Preferably, said fragment has a length of at least 50 nucleotides, more preferably of at least 60, 70, 80, 90, 100, 150, 200, 250, 500, 750, 1000 nucleotides. In a most preferred embodiment, said fragment has a length of at least 100 nucleotides. Preferably, the fragment is located in an exonic sequence of a pre-antisense lncRNA and, more preferably in a sequence combining at least two exons.

### Subject

As used herein, the terms “subject”, “individual” or “patient” are interchangeable and refer to an animal, preferably to a mammal, even more preferably to a human. However, the term “subject” can also refer to non-human animals, in particular mammals such as dogs, cats, horses, cows, pigs, sheep and non-human primates, among others, that are in need of diagnostic.

In a preferred embodiment of the above mentioned method, the subject is a man, preferably an adult man, more preferably a man of at least 50 years old, even more preferably a man of at least 60 years old.

In a particular embodiment of the above mentioned method, the subject has a family history of prostate cancer or other risk factors. In this case, the subject can be a man of at least 40 years old.

In another particular embodiment of the above mentioned method, the subject has relapse from a previous prostate cancer.

In a preferred embodiment of the above mentioned method, the subject is positive to a digital-rectal examination and/or to a prostate specific antigen (PSA) test.

In a preferred embodiment of the above mentioned method, the subject is periodically submitted to a prostate cancer diagnosis, preferably once a year, alternatively every two years or every three years.

In a particular embodiment of the above mentioned method, when the subject has a family history of prostate cancer or other risk factors, the subject is submitted to a prostate cancer diagnosis twice a year.

### Diagnosis Method

The above mentioned method comprises a step of determining the amount of at least one antisense lncRNA or a fragment thereof in a biological sample from the subject.

Prior to this step, the method may further comprise a step of obtaining or providing a sample from the subject.

The method may also comprise a step of preparing or extracting the nucleic acids, preferably the ribonucleic acids, from the sample. The amount of at least one antisense lncRNA may then be quantified in the preparation of RNAs extracted from the sample.

Sample

The term “biological sample”, as used herein, means any sample containing antisense lncRNAs derived from the subject. Examples of such biological samples include fluids such as blood, plasma, urine, seminal fluid samples or mixed urine and seminal fluid (first urine sample following ejaculation) as well as biopsies, organs, tissues or cell samples, in particular a prostate sample. Preferably, the biological sample is a blood or a urine sample. More preferably, the biological sample is a urine sample.

In one embodiment of the above mentioned method, the sample, preferably a urine sample, is obtained after an attentive digital-rectal examination (DRE) and/or a prostate specific antigen (PSA) test, preferably a urine sample is obtained if the PSA test and/or the digital-rectal examination were indicative of prostate cancer.

In a particular embodiment of the above mentioned method, the urine sample is obtained just after a digital-rectal examination. Indeed, the digital-rectal examination increases the amount of prostatic cells and prostatic cell fragments in the urine sample, thereby facilitating the detection of the antisense lncRNAs.

Of course, it should be understood that the present method can also be used on a sample obtained without being preceded by a digital-rectal examination and/or PSA test.

A urine preservative can be added to the sample, preferably a Norgen tubes preservative (ref. 18113 Norgen bioteck corporation), allowing the conservation of the sample at room temperature for 2 years.

Extraction

Several methods and kits are available for the person skilled in the art to extract the nucleic acids, and more particularly the ribonucleic acids, contained in the sample. For example, extraction may rely on lytic enzymes or chemical solutions or can be done with nucleic-acid-binding resins following the manufacturer's instructions.

In one embodiment of the above mentioned method, the cells, cell fragments and exosomes present in the sample, preferably a urine sample, are collected, preferably by centrifugation. A total nucleic acid extraction is then carried out. Non-limiting example are a phenol/chloroform or Trizol extraction methods. Total nucleic acid extraction may also be carried out using a solid phase band method on silica beads. Of course, it should be understood that numerous nucleic acid extraction methods exist and thus, that other methods can be used in accordance with the present invention.

As used herein, the term “exosome” refers to any kind of cell-derived vesicles present in biological fluids, including blood and urine. This term encompasses “microvesicles”, “epididimosomes”, “argosomes”, “exosome-like vesicles”, “microparticles”, “promininosomes”, “prostasomes”, “dexosomes”, “texosomes”, “dex”, “tex”, “archeosomes” “oncosomes”, “liposomes” and “micelles”.

Preferably, after total nucleic acid extraction, DNA is degraded so as to conserve only the RNA molecules. In particular, a deoxyribonuclease may be used to degrade DNA.

A variety of nucleic acids quantification techniques, well known by the skilled person, can be used to determine the amount of at least one antisense lncRNA from a biological sample, and in particular from the RNAs extracted from said sample. These techniques can be adapted in accordance with the type of sample, the sensitivity of the quantification desired, the amount of nucleic acid in the sample, and the like.

In particular, measurement of the amount of at least one antisense lncRNA can be direct or indirect. Indeed, the amount of an antisense lncRNAs can be directly quantified, preferably by hybridization, more preferably by hybridization of a labeled specific probe, still more preferably by hybridization of a fluorescent labeled specific probe immobilized directly or indirectly on a solid support, even more preferably by the Nanostring method.

Alternatively, the amount of an antisense lncRNA can be determined indirectly, especially after its conversion to cDNA, preferably by amplification, especially a quantitative amplification, more preferably by an amplification method coupled to real-time detection of the amplified products, even more preferably by quantitative RT-PCR.

Other methods of amplification include ligase chain reaction (LCR), transcription-mediated amplification (TMA), strand displacement amplification (SDA), nucleic acid sequence based amplification (NASBA), and microarray analysis. These methods are well known by the skilled person.

In a most preferred embodiment, the amount of at least one antisense lncRNA is determined by the nanostring method.

Labeled Probes

The method for determining the amount of at least one antisense lncRNA may involve probes, in particular labeled probes.

The term “label”, as used herein, refers to any atom or molecule that can be used to provide a quantifiable signal and that can be attached to a nucleic acid via a covalent bond or noncovalent interaction (e.g., through ionic or hydrogen bonding, or via immobilization, adsorption, or the like).

The probes of the present invention can be labeled by standard labeling techniques such as with a radiolabel, enzyme label, fluorescent label, biotin-avidin label, chemiluminescent label, and the like. After hybridization, the probes can be visualized using known methods. In particular, labels generally provide signals detectable by fluorescence, chemiluminescence, radioactivity, colorimetry, mass spectrometry, X-ray diffraction or absorption, magnetism, enzymatic activity, or the like.

Preferably, the detectable label may be a luminescent label. For example, fluorescent labels, bioluminescent labels, chemiluminescent labels, and colorimetric labels may be used in the practice of the invention, more preferably a fluorescent label.

The terms “fluorescent label”, “fluorophore”, “fluorogenic dye”, “fluorescent dye” as used herein are interchangeable and designate a functional group attached to a nucleic acid that will absorb energy of a specific wavelength and re-emit energy at a different, but equally specific, wavelength.

Fluorescent labels that can be used in the context of this invention include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. The fluorescent label can be made of a combination of fluorescent labels.

In one embodiment of the above described method, the probe is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

Quantitative RT-PCR

In one embodiment of the above mentioned method, the amount of at least one antisense lncRNA is quantified using an RNA reverse-transcription and amplification method. In such an embodiment, the RNA amplification method is coupled to a quantitative amplification such as real-time detection of the amplified products using fluorescence specific probes. Preferably, the amplification method is PCR. More preferably, the PCR is a quantitative PCR or a related method enabling detection in real-time of the amplified products.

Among the amplification methods, quantitative reverse transcription PCR (quantitative RT-PCR) is preferred.

As used herein, the terms “quantitative RT-PCR”, “qRT-PCR”, “Real time RT-PCR” and “quantitative Real time RT-PCR” are equivalent and can be used interchangeably. Likewise, the terms “quantitative PCR”, “qPCR”, “Real time PCR” and “quantitative real time PCR” are equivalent and can be used interchangeably.

Any of a variety of published quantitative RT-PCR protocols can be used (and modified as needed) for use in the present method. Suitable quantitative RT-PCR procedures include but are not limited to those presented in U.S. Pat. No. 5,618,703 and in U.S. Patent Application No. 2005/0048542, which are hereby incorporated by reference.

In a preferred embodiment of the above mentioned method, the quantitative RT-PCR includes two main steps, the reverse transcription (RT) of RNA in cDNA and the quantitative PCR (Polymerase Chain Reaction) amplification of the cDNA.

Quantitative RT-PCR can be performed by an uncoupled or by a coupled procedure. In an uncoupled quantitative RT-PCR, the reverse transcription is performed independently from the quantitative PCR amplification, in separate reactions. Whereas, in a coupled quantitative RT-PCR, the reverse transcription and the quantitative PCR amplification are performed in a single reaction tube using a common reaction mixture including both the reverse transcriptase and the DNA polymerase. The method of the invention encompasses all versions of quantitative RT-PCR.

The term “reaction mixture” or “master mix” or “master mixture” refers to an aqueous solution of constituents in a quantitative RT-PCR reaction that can be constant across different reactions. In case of a coupled procedure, an exemplary “quantitative RT-PCR reaction mixture” includes buffer, a mixture of deoxyribonucleoside triphosphates, reverse transcriptase, RT and PCR primers, probes, and DNA polymerase. In case of uncoupled procedure, two reaction mixtures are needed, a “RT reaction mixture” including, for example, buffer, a mixture of deoxyribonucleoside triphosphates, reverse transcriptase and RT primers, and a “quantitative PCR” or “PCR reaction mixture” including, for example, buffer, a mixture of deoxyribonucleoside triphosphates, PCR primers, probes, and DNA polymerase.

The RNA or cDNA templates have to be added to theses reaction mixtures.

Reverse Transcription

The reverse transcription includes three basic steps: (1) denaturation of RNA, (2) hybridizing of the RT primers, (3) synthesis of cDNA.

The term “RT primers”, as used herein, mean oligonucleotide primers that anneal to RNA and allow reverse transcriptase to elongate it into cDNA. Two different types of RT primers may be used, non-specific oligonucleotide RT primers or sequence specific oligonuclotide RT primers.

Using non-specific oligonucleotide RT primers such as Oligo dT (specific of polyA tail of RNAs) or random primers (for example hexamers) allows the reverse transcription of the most of the RNA present in the sample.

Using sequence specific RT primers (primers complementary to a sequence of the target RNA) allows the reverse-transcription of only the RNAs of interest. In particular, a sequence specific RT primer can be a primer which has a nucleic acid sequence complementary to the antisense lncRNA target but not to the corresponding mRNA, allowing the specific reverse transcription of the antisense lncRNA. However, to conduct a reverse transcription with a sequence specific RT primer, a multiplexing (use of several primers targeting several distinct RNA molecules) or multiple RT reactions are needed. In the latter case a greater amount of RNA is needed.

Preferably, the reverse transcription is non-specific, more preferably the RT primers used are random primers.

Reverse transcriptase enzymatic activity provides a cDNA transcript from an RNA template. There are many reverse transcriptases that are commercially available (e.g., Moloney Murine Leukemia Virus (M-MuLV) Reverse Transcriptase from New England Biolabs, Inc., Beverly, Mass.; HIV reverse transcriptase from Ambion, Inc., Austin, Tex.). The methods of the invention are not limited to any particular enzyme, although some enzymes may be preferable under specific conditions. The two most commonly used reverse transcriptases are avilo myeloblastosis virus reverse transcriptase (AMV-RT) and Moloney murine leukemia virus reverse transcriptase (MLV-RT).

Prior to the quantitative amplification by PCR, in case of an uncoupled procedure:
the reverse transcription may further comprises a step of RNA degradation, for example by addition of an RNA nuclease to the reaction mixture; and/or
the reverse transcription may further comprise a step of inactivating the reverse transcriptase.

Quantitative PCR

Quantitative PCR allows quantification of reaction products for each sample per cycle. Commonly used instrumentation and software products perform the quantification calculations automatically. The PCR process generally consists in the repetition of a sequence of temperature changes (or cycle) conducted by a thermal cycler. Usually, 25 to 50 cycles are needed.

These cycles normally consist of three stages: the first (denaturation), at around 95° C., allows the separation of the nucleic acids double chain; the second (alignment), at a temperature of around 50-60° C., allows the binding of the PCR primers with the DNA template; the third (elongation), at between 68-72° C., facilitates the polymerization carried out by the DNA polymerase. Due to the small size of the fragments amplified in quantitative PCR, the last step is usually omitted as the enzyme is able to increase their number during the change between the alignment stage and the denaturing stage. Preferably, a step of fluorescence measurement may be added. The temperatures and the timings used for each cycle depend on a wide variety of parameters, such as the DNA polymerase used, the concentration of divalent ions and deoxyribonucleotides in the reaction mixture and the binding temperature of the primers.

The recent advancement in PCR instrumentation technology (e.g., Cepheid's Smart Cycler® II), allows the simultaneous detection and quantification of different fluorescent signals in different channels (PCR multiplex) in real-time. In addition, the latest generation of thermal cyclers are designed to maximize fluorescent dye excitation providing a more accurate means of detecting fluorescence. Thus, multiple amplification products can be assessed in the same reaction mixture and quantified more accurately. For example, the amounts of at least three antisense lncRNA as defined above can be simultaneously determined by quantitative RT-PCR.

The term “PCR primer” or “primer pair”, as used herein, are equivalent and mean a pair of oligonucleotide primers that are complementary to the sequences within a target cDNA sequence in a PCR. The primer pair consists of a forward primer and a reverse primer which nucleic acid sequences are complementary to a minus (reverse) and a plus (forward) strand of the double stranded cDNA fragment of interest, respectively.

Primers of a primer pair should have similar hybridizing conditions and in particular similar melting temperatures since annealing in a PCR occurs for both simultaneously. A primer with a Tm (melting temperature) significantly higher than the reaction's annealing temperature may mishybridize and extend at an incorrect location along the DNA sequence, while a primer with a Tm significantly lower than the annealing temperature may fail to anneal and will not extend at all. Primer sequences also need to be chosen to uniquely select for a region of DNA, avoiding the possibility of mishybridization to a similar sequence nearby.

It is not necessary that every nucleotide of the PCR primers anneal to the template to allow the amplification of the cDNA. For example, a non-complementary nucleotide fragment may be attached to the 5′ end of the PCR primers with the remainder of the primer sequence being complementary to the cDNA. Alternatively, non-complementary bases can be interspersed into the PCR primer, provided that the primer sequence has sufficient complementarity with the cDNA. Thus, the embodiments of the invention contemplate variants of the PCR primers described herein.

In one particular embodiment of the above mentioned method, the primers span the 3′ region of a first exon and the 5′ region of a second exon, so as such primers can only amplify sequences which the first and second exon have been spliced into a contiguous position (i.e. by removing an intervening intronic sequence). Knowing the sequences of the exon boundaries, as well as those of the different exons, the primers which can be designed and used in the context of the present invention can be readily determined by a person of ordinary skill in the art to which the present invention pertains.

In a preferred embodiment of the above mentioned method, a primer pair consists in primers that are complementary to either exon or intron sequences of the pre-antisense lncRNA, but are obligatory out of exons of the corresponding pre-mRNA. Such primers cannot hybridize to the cDNA reversed transcript from said mRNA or any sequence deriving from it. Preferably, the primers are complementary to exon sequences of the pre-antisense lncRNA and are obligatory out of exons of the corresponding pre-mRNA.

This primer pair allows the specific amplification of the cDNA reversed transcript from the antisense lncRNA.

Many DNA polymerases suitable for quantitative PCR are commercially available (e.g., Taq and T7 DNA polymerases from New England Biolabs, Inc.; Pfu DNA polymerase from Promega, Inc., Madison, Wis.). The method of the invention is not limited to any particular enzyme, although some enzymes may be preferable under specific conditions.

The term “probe”, in the context of the quantitative RT-PCR, refers to an oligonucleotide that hybridizes to a target sequence situated between the annealing sites of the two primers of the primer pair. The probe includes a detectable label, e.g., a fluorophore (Texas-Red®, Fluorescein isothiocyanate, etc.) that can be covalently attached directly to the probe oligonucleotide, e.g., located at the probe's 5′ end or at the probe's 3′ end. The probe may also include a quencher. A probe includes about 8 nucleotides, about 10 nucleotides, about 15 nucleotides, about 20 nucleotides, about 30 nucleotides, about 40 nucleotides, or about 50 nucleotides. In some embodiments, a probe includes from about 8 nucleotides to about 15 nucleotides.

The term “quenching”, as used herein, refers to a decrease in fluorescence of a fluorescent detectable label caused by energy transfer associated with a quencher moiety, regardless of the mechanism. Suitable quencher moiety is for example Black Hole Quencher™ (Biosearch Technologies, Novato, Calif.) and Iowa Black (Integrated DNA Technologies, Coralville, Iowa).

Four different probe systems are in current use for quantitative PCR-Molecular Beacons (Sigma-Genosys, Inc., The Woodlands, Tex.), Scorpions® (DxS Ltd., Manchester, UK), SYBR® Green (Molecular Probes, Eugene, Oreg.), and TaqMan® (Applied Biosystems, Foster City, Calif.). These four systems employ fluorescent labels that the instrumentation detects and the software interprets levels of fluorescence.

SYBR® Green is a fluorescent dye that only strongly fluoresces when bound to double stranded DNA. SYBR® green assay is a quick method, particularly useful for detection.

Molecular Beacons, Scorpions®, and TaqMan® utilize Förster Resonance Energy Transfer (FRET) by coupling a fluorescent label with a quencher moiety. A fluorescent label is covalently bound to the 5′ end of an oligonucleotide probe, while the 3′ end has a quencher moiety attached. These oligonucleotide probes are site specific to hybridize to the amplified product. Preferably, the oligonucleotide probes are designed to hybridize to a central region of the amplified product.

For TaqMan® assays, the 5′→3′ exonuclease activity of the DNA polymerase cleaves the probe during the elongation cycle. Due to the cleavage of the probe, the quencher moiety is no longer coupled to the fluorescence label and cannot quench fluorescence. One molecule of reporter fluorescent dye is liberated for each new molecule synthesized, and detection of the unquenched reporter fluorescent dye provides the basis for quantitative interpretation of the data. Fluorescence thus represents replicating DNA.

Similarly, Molecular Beacons utilizes an oligonucleotide probe with a fluorescent label attached to the 5′ end and a quencher moiety attached to the 3′ end. When free in solution, the Molecular Beacons oligonucleotide probe forms a hairpin structure. In the hairpin structure, the quencher moiety is able to quench fluorescence due to FRET. However, during PCR, the oligonucleotide probe unfolds and hybridizes to its complementary DNA, and the quencher is no longer close enough to the fluorescent label to quench fluorescence. Thus, fluorescence reports the hybridization between an oligonucleotide probe and its specific target cDNA.

Scorpions® also utilize an oligonucleotide probe with a fluorescent label attached to the 5′ end and a quencher moiety attached to the 3′ end in a hairpin structure free in solution. However, the Scorpions® oligonucleotide probe also serves as a primer. The Scorpions® oligonucleotide probe/primer extends from the hairpin loop structure and hybridizes to the target. The Scorpions® oligonucleotide probe/primer fluoresces, and the DNA polymerase extends the target DNA from the primer. Thus, the probe detects the extension product, which is its own primer-unimolecular rearrangement. Thereby, the fluorescence reports the extension and thus copy number of reaction product.

In a preferred embodiment, the present method employs a SYBR® Green fluorescent dye. Alternatively, the present method employs TaqMan®-style probes, i.e. dual-labeled probes aimed to fluoresce upon 5′→3′ exonuclease activity.

Analysis of the Quantitative RT-PCR Results

RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). The system consists of a thermocycler, laser, charge-coupled device (CCD), camera and computer. The system includes software for running the instrument and for analyzing the data (determining the quantity of amplification product based upon the fluorescence data).

To minimize errors and the effect of sample-to-sample variations, quantitative RT-PCR is usually performed using an internal reference. The ideal internal reference is expressed at a constant level among different samples, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A standard curve can be generated from a cDNA of known concentration. The standard curve can then be used to determine absolute or relative cDNA levels.

The comparative cycle threshold (Ct) method, also known as the 2-ΔΔCt method, allows to quantify cDNA levels. Fluorescence values are recorded during every cycle and represent the amount of product amplified to that point in the amplification reaction. The point when the fluorescent signal is first recorded as statistically significant is the threshold cycle (Ct).

The Ct method compares a test reaction with a control or calibrator sample. The Ct values of both the control/calibrator sample and the test sample are normalized. In an embodiment of the invention, the Ct values may be normalized to an arbitrary cutoff (e.g. 20-22).

The Ct method can also be described by the formula ΔΔCt=ΔCttest sample−ΔCt reference sample. The amplification efficiencies of the test sample and the reference sample must be about the same for the formula to operate. Amplification efficiencies can be determined by a comparison of the samples with template dilution. The amplification efficiency is about the same when a plot of cDNA dilution versus ΔCt approximates zero.

In one embodiment of the above mentioned method, the amount of at least one antisense lncRNA is determined by quantitative RT-PCR, wherein said quantitative RT-PCR comprises the steps of:
reverse transcription of the RNAs present in the sample into cDNAs; and
amplification of the cDNA reverse transcript from the at least one antisense lncRNA or a part of said cDNA by quantitative PCR, wherein said part has a length of at least 50 nucleotides.

Preferably, said reverse transcription comprises a step of contacting said RNAs with RT primers, more preferably random primers.

Preferably, said amplification comprises a step of contacting said cDNA with at least one primer pair, more preferably said amplification further comprises the step of contacting said cDNA with at least one labeled probe. The labeled probe of the invention is complementary to either exon or intron sequences of the pre-antisense lncRNA, but is obligatory out of exons of the corresponding sense pre-mRNA. More preferably, the labeled probe is complementary to exon sequences of the pre-antisense lncRNA and is obligatory out of exons of the corresponding pre-mRNA. Thus, the labeled probe of the invention is able to label the pre-antisense lncRNA but not the corresponding sense pre-mRNA.

Preferably, the at least one primer pair consists in primers that are complementary to either exon or intron sequences of the pre-antisense lncRNA, but are obligatory out of exons of the corresponding sense pre-mRNA. More preferably, the primers are complementary to exon sequences of the pre-antisense lncRNA and are obligatory out of exons of the corresponding pre-mRNA. Thus, primer pairs of the invention are able to amplify the pre-antisense lncRNA but not the corresponding sense pre-mRNA.

Even more preferably, said at least one primer pair is selected from the group consisting of:
the primers of SEQ ID No.4 and SEQ ID No.5, wherein said primers are specific of the cDNA reverse transcript from asABCC4; and
the primers of SEQ ID No.6 and SEQ ID No.7, wherein said primers are specific of the cDNA reverse transcript from asABCC4; and
the primers of SEQ ID No.8 and SEQ ID No.9, wherein said primers are specific of the cDNA reverse transcript from asPDLIM5; and
the primers of SEQ ID No.10 and SEQ ID No.11, wherein said primers are specific of the cDNA reverse transcript from asPDLIM5; and
the primers of SEQ ID No.12 and SEQ ID No.13, wherein said primers are specific of the cDNA reverse transcript from asPDLIM5; and
the primers of SEQ ID No.14 and SEQ ID No.15, wherein said primers are specific of the cDNA reverse transcript from asPDLIM5; and
the primers of SEQ ID No.16 and SEQ ID No.17, wherein said primers are specific of the cDNA reverse transcript from asFXL7; and
the primers of SEQ ID No.18 and SEQ ID No.19, wherein said primers are specific of the cDNA reverse transcript from asFXL7; and
the primers of SEQ ID No.20 and SEQ ID No.21, wherein said primers are specific of the cDNA reverse transcript from asFXL7.

Preferably, said amplification further comprises a step of contacting said cDNAs with a SYBR® Green fluorescent dye, alternatively said amplification further comprises a step of contacting said cDNAs with at least a dual labeled probe, i.e. a probe with a fluorescent label and a quencher moiety, said at least one dual labeled probe being complementary to a sequence of cDNA localized between the two primers of the primer pair. More preferably, the dual labeled probe fluoresce upon 5′→3′ exonuclease activity.

Preferably, when said at least one labeled probe comprises more than one probe, each probe has a different label, i.e. fluoresces at a different wavelength.

Alternatively, a combination of two different primer pairs specific of the same antisense lncRNA are used to amplify the cDNA by quantitative PCR. Preferably, the two primer pairs of this combination are used in separate PCR mixtures, alternatively they can be used in the same PCR mixture. For instance, this combination of two primer pairs can be selected from the group consisting of: the primer pair of SEQ ID No.4 and SEQ ID No.5 and the primer pair of SEQ ID No.6 and SEQ ID No.7, the primer pair of SEQ ID No.8 and SEQ ID No.9 and the primer pair of SEQ ID No.10 and SEQ ID No.11, the primer pair of SEQ ID No.8 and SEQ ID No.9 and the primer pair of SEQ ID No.12 and SEQ ID No.13; the primer pair of SEQ ID No.8 and SEQ ID No.9 and the primer pair of SEQ ID No.14 and SEQ ID No.15, the primer pair of SEQ ID No.10 and SEQ ID No.11 and the primer pair of SEQ ID No.12 and SEQ ID No.13, the primer pair of SEQ ID No.10 and SEQ ID No.11 and the primer pair of SEQ ID No.14 and SEQ ID No.15, the primer pair of SEQ ID No.12 and SEQ ID No.13 and the primer pair of SEQ ID No.14 and SEQ ID No.15, the primer pair of SEQ ID No.16 and SEQ ID No.17 and the primer pair of SEQ ID No.18 and SEQ ID No.19, the primer pair of SEQ ID No.15 and SEQ ID No.16 and the primer pair of SEQ ID No.20 and SEQ ID No.21, and the primer pair of SEQ ID No.18 and SEQ ID No.19 and the primer pair of SEQ ID No.20 and SEQ ID No.21.

In a particular embodiment of the above mentioned method, the amount of at least two antisense lncRNA is determined by quantitative RT-PCR, wherein said quantitative RT-PCR comprises the steps of:
reverse transcription of the RNAs present in the sample into cDNAs.
amplification of the cDNA reverse transcripts from the at least two antisense lncRNAs or a part of said cDNAs by quantitative PCR, wherein said part has a length of at least 50 nucleotides and wherein said amplification comprises the step of contacting said cDNAs with at least two primer pairs.

Preferably, said amplification further comprises the step of contacting said cDNAs with at least two labeled probes, more preferably dual labeled probes, even more preferably dual labeled probe that fluoresce upon 5′→3′ exonuclease activity, wherein each of said at least two dual labeled probes being complementary to a sequence of a cDNA localized between the two primers of one of the at least two primer pairs.

Preferably, said combination of at least two primer pairs are used in the same PCR mixture. Alternatively they can be used in different PCR mixture. For instance, this combination of at least two primer pairs can be selected from the group consisting of: the primer pair of SEQ ID No.4 and SEQ ID No.5 and the primer pair of SEQ ID No.8 and SEQ ID No.9, the primer pair of SEQ ID No.4 and SEQ ID No.5 and the primer pair of SEQ ID No.10 and SEQ ID No.11, the primer pair of SEQ ID No.4 and SEQ ID No.5 and the primer pair of SEQ ID No.12 and SEQ ID No.13, the primer pair of SEQ ID No.4 and SEQ ID No.5 and the primer pair of SEQ ID No.14 and SEQ ID No.15, the primer pair of SEQ ID No.4 and SEQ ID No.5 and the primer pair of SEQ ID No.16 and SEQ ID No.17, the primer pair of SEQ ID No.4 and SEQ ID No.5 and the primer pair of SEQ ID No.18 and SEQ ID No.19, the primer pair of SEQ ID No.4 and SEQ ID No.5 and the primer pair of SEQ ID No.20 and SEQ ID No.21, the primer pair of SEQ ID No.6 and SEQ ID No.7 and the primer pair of SEQ ID No.8 and SEQ ID No.9, the primer pair of SEQ ID No.6 and SEQ ID No.7 and the primer pair of SEQ ID No.10 and SEQ ID No.11, the primer pair of SEQ ID No.6 and SEQ ID No.7 and the primer pair of SEQ ID No.12 and SEQ ID No.13, the primer pair of SEQ ID No.6 and SEQ ID No.7 and the primer pair of SEQ ID No.14 and SEQ ID No.15, the primer pair of SEQ ID No.6 and SEQ ID No.7 and the primer pair of SEQ ID No.16 and SEQ ID No.17, the primer pair of SEQ ID No.6 and SEQ ID No.7 and the primer pair of SEQ ID No.18 and SEQ ID No.19, the primer pair of SEQ ID No.6 and SEQ ID No.7 and the primer pair of SEQ ID No.20 and SEQ ID No.21, the primer pair of SEQ ID No.8 and SEQ ID No.9 and the primer pair of SEQ ID No.16 and SEQ ID No.17, the primer pair of SEQ ID No.8 and SEQ ID No.9 and the primer pair of SEQ ID No.18 and SEQ ID No.19, the primer pair of SEQ ID No.8 and SEQ ID No.9 and the primer pair of SEQ ID No.20 and SEQ ID No.21, the primer pair of SEQ ID No.10 and SEQ ID No.11 and the primer pair of SEQ ID No.16 and SEQ ID No.17, the primer pair of SEQ ID No.10 and SEQ ID No.11 and the primer pair of SEQ ID No.18 and SEQ ID No.19, the primer pair of SEQ ID No.10 and SEQ ID No.11 and the primer pair of SEQ ID No.20 and SEQ ID No.21, the primer pair of SEQ ID No.12 and SEQ ID No.13 and the primer pair of SEQ ID No.16 and SEQ ID No.17, the primer pair of SEQ ID No.12 and SEQ ID No.13 and the primer pair of SEQ ID No.18 and SEQ ID No.19, the primer pair of SEQ ID No.12 and SEQ ID No.13 and the primer pair of SEQ ID No.20 and SEQ ID No.21, the primer pair of SEQ ID No.14 and SEQ ID No.15 and the primer pair of SEQ ID No.16 and SEQ ID No.17, the primer pair of SEQ ID No.14 and SEQ ID No.15 and the primer pair of SEQ ID No.18 and SEQ ID No.19, and the primer pair of SEQ ID No.14 and SEQ ID No.15 and the primer pair of SEQ ID No.20 and SEQ ID No.21.

Preferably, a combination of four different primer pairs specific of two different antisense lncRNAs are used to amplify the cDNAs by quantitative PCR. For instance, this combination of at least four primer pairs can be selected from the group consisting of: the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.1, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.16 and SEQ ID No.7 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.10 and SEQ ID No.11 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.10 and SEQ ID No.11 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.10 and SEQ ID No.11 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.20 and SEQ ID No.21, and the primer pair of SEQ ID No.12 and SEQ ID No.13 and the primer pair of SEQ ID No.14 and SEQ ID No.15 and the primer pair of SEQ ID No.18 and SEQ ID No.19 and the primer pair of SEQ ID No.20 and SEQ ID No.21.

In a preferred embodiment of the above mentioned method, the amount of at least three antisense lncRNAs is determined by quantitative RT-PCR, wherein said quantitative RT-PCR comprises the steps of:
reverse transcription of the RNAs present in the sample into cDNAs.
amplification of the cDNA reverse transcripts from the at least three antisense lncRNAs or a part of said cDNAs by quantitative PCR, wherein said part has a length of at least 50 nucleotides and wherein said amplification comprises the step of contacting said cDNAs with at least three primer pairs.

Preferably, said combination of at least three primer pairs are used in the same PCR mixture, alternatively they can be used in different PCR mixture. For instance, this combination of at least three primer pairs can be selected from the group consisting of: the primer pair of SEQ ID No.4 and SEQ ID No.5 and the primer pair of SEQ ID No.8 and SEQ ID No.9 and the primer pair of SEQ ID No.16 and SEQ ID No.17, the primer pair of SEQ ID No.4 and SEQ ID No.5 and the primer pair of SEQ ID No.8 and SEQ ID No.9 and the primer pair of SEQ ID No.18 and SEQ ID No.19, the primer pair of SEQ ID No.4 and SEQ ID No.5 and the primer pair of SEQ ID No.8 and SEQ ID No.9 and the primer pair of SEQ ID No.20 and SEQ ID No.21, the primer pair of SEQ ID No.4 and SEQ ID No.5 and the primer pair of SEQ ID No.10 and SEQ ID No.11 and the primer pair of SEQ ID No.16 and SEQ ID No.17, the primer pair of SEQ ID No.4 and SEQ ID No.5 and the primer pair of SEQ ID No.10 and SEQ ID No.11 and the primer pair of SEQ ID No.18 and SEQ ID No.19, the primer pair of SEQ ID No.4 and SEQ ID No.5 and the primer pair of SEQ ID No.10 and SEQ ID No.11 and the primer pair of SEQ ID No.20 and SEQ ID No.21, the primer pair of SEQ ID No.4 and SEQ ID No.5 and the primer pair of SEQ ID No.12 and SEQ ID No.13 and the primer pair of SEQ ID No.16 and SEQ ID No.17, the primer pair of SEQ ID No.4 and SEQ ID No.5 and the primer pair of SEQ ID No.12 and SEQ ID No.13 and the primer pair of SEQ ID No.18 and SEQ ID No.19, the primer pair of SEQ ID No.4 and SEQ ID No.5 and the primer pair of SEQ ID No.11 and SEQ ID No.12 and the primer pair of SEQ ID No.20 and SEQ ID No.21, the primer pair of SEQ ID No.4 and SEQ ID No.5 and the primer pair of SEQ ID No.14 and SEQ ID No.15 and the primer pair of SEQ ID No.16 and SEQ ID No.17, the primer pair of SEQ ID No.4 and SEQ ID No.5 and the primer pair of SEQ ID No.14 and SEQ ID No.15 and the primer pair of SEQ ID No.18 and SEQ ID No.19, the primer pair of SEQ ID No.4 and SEQ ID No.5 and the primer pair of SEQ ID No.14 and SEQ ID No.15 and the primer pair of SEQ ID No.20 and SEQ ID No.21, the primer pair of SEQ ID No.6 and SEQ ID No.7 and the primer pair of SEQ ID No.8 and SEQ ID No.9 and the primer pair of SEQ ID No.16 and SEQ ID No.17, the primer pair of SEQ ID No.6 and SEQ ID No.7 and the primer pair of SEQ ID No.8 and SEQ ID No.9 and the primer pair of SEQ ID No.18 and SEQ ID No.19, the primer pair of SEQ ID No.6 and SEQ ID No.7 and the primer pair of SEQ ID No.8 and SEQ ID No.9 and the primer pair of SEQ ID No.20 and SEQ ID No.21, the primer pair of SEQ ID No.6 and SEQ ID No.7 and the primer pair of SEQ ID No.10 and SEQ ID No.11 and the primer pair of SEQ ID No.16 and SEQ ID No.17, the primer pair of SEQ ID No.6 and SEQ ID No.7 and the primer pair of SEQ ID No.10 and SEQ ID No.11 and the primer pair of SEQ ID No.18 and SEQ ID No.19, the primer pair of SEQ ID No.6 and SEQ ID No.7 and the primer pair of SEQ ID No.10 and SEQ ID No.11 and the primer pair of SEQ ID No.20 and SEQ ID No.21, the primer pair of SEQ ID No.6 and SEQ ID No.7 and the primer pair of SEQ ID No.12 and SEQ ID No.13 and the primer pair of SEQ ID No.16 and SEQ ID No.17, the primer pair of SEQ ID No.6 and SEQ ID No.7 and the primer pair of SEQ ID No.12 and SEQ ID No.13 and the primer pair of SEQ ID No.18 and SEQ ID No.19, the primer pair of SEQ ID No.6 and SEQ ID No.7 and the primer pair of SEQ ID No.11 and SEQ ID No.12 and the primer pair of SEQ ID No.20 and SEQ ID No.21, the primer pair of SEQ ID No.6 and SEQ ID No.7 and the primer pair of SEQ ID No.14 and SEQ ID No.15 and the primer pair of SEQ ID No.16 and SEQ ID No.17, the primer pair of SEQ ID No.6 and SEQ ID No.7 and the primer pair of SEQ ID No.14 and SEQ ID No.15 and the primer pair of SEQ ID No.18 and SEQ ID No.19, and the primer pair of SEQ ID No.6 and SEQ ID No.7 and the primer pair of SEQ ID No.14 and SEQ ID No.15 and the primer pair of SEQ ID No.20 and SEQ ID No.21.

Preferably, said amplification further comprises the step of contacting said cDNAs with at least three labeled probe, more preferably dual labeled probes, even more preferably dual labeled probe that fluoresce upon 5′→3′ exonuclease activity, wherein each of said at least three dual labeled probes being complementary to a sequence of cDNA localized between the two primers of one of the at least three primer pairs.

Preferably, a combination of six different primer pairs specific of three different antisense lncRNAs are used to amplify the cDNAs by quantitative PCR. For instance, this combination of at least six primer pairs can be selected from the group consisting of: the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.20 and SEQ ID No.21, and the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.18 and SEQ ID No.19 and the primer pair of SEQ ID No.20 and SEQ ID No.21.

The above mentioned method may further comprise additional primer pairs, preferably primer pairs targeting other antisense lncRNA which are diagnosis marker for prostate cancer, more preferably primer pairs for PCA3 quantitative amplification.

Hybridization Method

In one embodiment of the above mentioned method, the amount of at least one antisense lncRNA is determined using an RNA hybridization method. In such an embodiment, the RNA hybridization method is coupled to the detection of the hybridized RNA using a probe, preferably a labeled specific probe. Even more preferably, the specific probe is labeled by a fluorophore.

When the amounts of several antisense lncRNAs are determined simultaneously, each probe is linked to a different label.

The complex probe/antisense lncRNA may be bound on a solid support, allowing to wash the reaction of the unbound probes.

After extraction of the RNA from the sample, the determination of the amount of at least one antisense lncRNA by an RNA hybridization method comprises the following steps:
Contacting at least one labeled specific probe with the extracted RNAs in conditions suitable for hybridization of said probe with its antisense lncRNA target; and
Quantification of the signal emitted by the probe.

Optionally, the RNAs are denatured prior to the contacting step, thereby removing the RNA secondary structure.

In a preferred embodiment, the at least one antisense lncRNA is immobilized on a solid support. Optionally, after immobilization of the at least one antisense lncRNA and the step of contacting, the method described above comprises a washing step, thereby removing the unbound probes.

The antisense lncRNAs may be directly immobilized on a solid support prior to be contacted with the probes. In such an embodiment, the amount of at least one antisense lncRNA is preferably determined by Northern blot.

Alternatively, the antisense lncRNAs may be indirectly immobilized on a solid support through its hybridization to its specific probe. Particularly, the sample may be contacted with probes already immobilized on a solid support. Preferably, the complex antisense lncRNA/probe is immobilized on a solid support after hybridization.

Preferably, the probe is linked to a molecule that helps the probe to bind a solid support. More preferably said molecule is biotin, thereby the probe and its complementary antisense lncRNA are immobilized on the solid support.

Examples of solid supports suitable for the invention include, but are not limited to, plastics such as polycarbonate, complex carbohydrates such as agarose and sepharose, and acrylic resins such as polyacrylamide and latex beads. Techniques for coupling nucleic acid and/or nucleic acid probes to such solid supports are well known in the art.

In another particular embodiment, the amount of at least one antisense lncRNA is determined by a LNA (Lock nucleic acid) method (see for example the techniques developed by Exiqon).

Preferably, the amount of at least one antisense lncRNA is determined by the Nanostring method.

Nanostring Method

The nanostring method is a hybridization method that allows to quantify RNA without requiring linear (array) nor exponential (PCR) amplification. It is a very sensitive method, since only 10 ng of RNA are needed to perform it, allowing analysis of quantity limited biological samples, such as urine. The restricted number of sample manipulation steps together with the absence of enzymatic reaction allows precise and physiologically correct quantifications. This method is also extremely flexible since it can be applied to various types of samples.

The Nanostring method necessitates the use of a pair of probes specifically designed for each antisense lncRNA target.

The first probe, called the capture-probe, specifically hybridizes the antisense lncRNA target and binds it to a solid support, preferably a counting stand. Preferably, the capture probe is linked to a molecule that allows the probe to bind the solid support, more preferably said molecule is biotin, thereby immobilizing the antisense lncRNA of interest onto the counting stand.

The second probe, called the reporter-probe, specifically hybridizes the antisense lncRNA of interest and is linked to a label that allows the detection and quantification of the antisens lncRNA. Preferably, this label is a fluorescent label. More preferably the label is made of a combination of fluorochromes. Even more preferably the label is made of a combination of 6 fluorochromes chosen among 4 fluorochromes of different colors, defining a code specific to each target antisense lncRNA. This color code confers to the technique a very high sensitivity and enables the analysis of quantity-limited biological samples. When several reporter probes are used simultaneously to determine the amounts of several antisense lncRNA, each reporter probe is linked to a different label, preferably a different combination of 6 fluorochromes chosen among 4 fluorochromes of different colors.

Preferably, the fluorescence is analyzed by an nCounter, an optical system that is capable to identify the color codes.

Preferably, the determination of the amount of at least one antisense lncRNA by the Nanostring method comprises the following steps:
Contacting the sample with the capture and the reporter probes, in conditions suitable for hybridization with its antisense lncRNA target; and
Elimination of the excess probes.
Alignment and immobilization of the probes/target complexes into the solid support.
Collection of the signal by the Nanostring device.

More preferably, the determination of the amount of at least one antisense lncRNA by the Nanostring method comprises the following steps:
Contacting the sample with the capture and the reporter probes, in conditions suitable for hybridization with its antisense lncRNA target; and
Elimination of the excess probes is achieved on a Nanostring Prep Station following an high sensitivity mode containing a dual purification process. The Nanostring prep station is robotic liquid handling system dedicated to purification of molecules coupled to probes and specially to capture probes and reporter probes. As capture probes and reporter probes contain also a specific tail (generic and specific sequence to the capture probe, idem for the reporter probes), two types of dedicated magnetic beads supplied by Nano string will be used in this main step. First, magnetic bead coupled with sequences complementary to tails of capture probes are used to purify capture probes hybridized or not to targets of interest. After washes, purified molecules are mixed with another magnetic beads that are coupled to sequences complementary to reporter probes tails. This step selects molecules bound to the set of capture probes and reporter probes. Washes are applied to by the Nanostring Prep station to ensure correct purification of hybrids.
Next, dually purified molecules are injected in a solid device: a 12 slot Nanostring Cartridge. This device contains 12 slots meaning that 12 samples can be analyzed in the meantime. Per slot, purified and injected molecules are immobilized thanks to a streptavidin coating. A magnetic fields is also applied to align the probes/target complexes into the solid device.
Collection of the signal, in particular by the Digital Analyzer on the sample Cartridges. Color codes on the surface of the cartridge are counted and tabulated for each target molecule.

Optionally, the ARNs are denatured prior to the contacting step, thereby removing the ARN secondary structure.

The Nanostring probes hybridize to their antisense lncRNA target with a part of their nucleotide sequence which is of a length of between about 40 and about 60 nucleotides, preferably of a length of about 50 nucleotides. The capture probe may further comprise a sequence that allows to link it to biotin. The reporter probe may further comprise a sequence that allows to link it to a six fluorochromes code. The color code is specific to each lot of production done by Nanostring. To deconvoluate signals and assign color code to targets, Nanostring provides a correspondence file specific to each kit.

Preferably, the sequences of the Nanostring probes of a pair of probes target different parts of their antisense lncRNA and these parts do not overlap.

Optionally, the sequences of the Nanostring probes of a pair of probes may be chosen so as they are not distant of more than 5 nucleotides on their antisense lncRNA target, preferably they are not distant of more than 10, 15, 20, 30, 40, 50, 100 nucleotides. In a most preferred embodiment, the Nanostring probes of a pair of probes are directly consecutive.

In one embodiment of the above mentioned method, the amount of at least one antisense lncRNA is determined by the Nanostring method, wherein said Nanostring method comprises the step of contacting the sample with at least one capture-probe and one reporter probe, wherein said capture probe is able to specifically hybridize the at least one antisense lncRNA and is preferably linked to a molecule that helps to immobilize said at least one antisense lncRNA onto a solid support, more preferably said molecule is biotin, and wherein said reporter-probe is able to specifically hybridize the at least one antisense lncRNA and is linked to a fluorescent label, preferably a combination of fluorophores, more preferably a combination of 6 fluorochromes chosen among 4 fluorochromes of different colors defining a code specific to said at least one antisense lncRNA.

Preferably, said at least one capture-probe and one reporter probe are selected from the group consisting of:
the probes of SEQ ID No.22 and SEQ ID No.23, wherein said probes are complementary to the antisense lncRNA of SEQ ID No.1; and
the probes of SEQ ID No.24 and SEQ ID No.25, wherein said probes are complementary to the antisense lncRNA of SEQ ID No.2; and
the probes of SEQ ID No.26 and SEQ ID No.27, wherein said probes are complementary to the antisense lncRNA of SEQ ID No.3;

In a particular embodiment of the above mentioned method, the amount of at least two antisense lncRNAs are determined by the Nanostring method, wherein said Nanostring method comprises the step of contacting the sample with a combination of at least two pairs of probes, said combination comprising two of the pairs of probes listed above. For instance, the combination can be selected from the group consisting of the pair of probes of SEQ ID No.22 and SEQ ID No.23 and the pair of probes consisted of SEQ ID No.24 and SEQ ID No.25, or the pair of probes of SEQ ID No.22 and SEQ ID No.23 and the pair of probes consisted of SEQ ID No.26 and SEQ ID No.27, or the pair of probes of SEQ ID No.24 and SEQ ID No.25 and the pair of probes consisted of SEQ ID No.26 and SEQ ID No.27.

In a preferred embodiment of the above mentioned method, the amount of at least three antisense lncRNAs are determined by the Nanostring method, wherein said Nanostring method comprises the step of contacting the sample with a combination of at least three pairs of probes, said combination comprising the three pairs of probes listed above.

The above mentioned combination may further comprise additional pairs of probes, preferably pairs of probes targeting other antisense lncRNAs which are diagnosis markers for prostate cancer, more preferably pairs of probes for PCA3 quantification by the Nanostring method.

The amount of an antisense lncRNA can also be determined from a biological sample by serial analysis of gene expression (SAGE), immunoassay, mass spectrometry, and any sequencing-based methods known in the art.

### Comparison to a Control Reference Value and Ttreatment

In an embodiment of the above mentioned methods, the method further comprises the step of comparing the amount of antisense lncRNA in a biological sample to a reference amount. In particular, the reference amount can be the amount of the same antisense lncRNA in a normal sample, i.e. a sample from a subject that did not have a prostate cancer. The normal sample may be obtained from the subject affected with the prostate cancer before the beginning of the disease or from another subject, preferably a normal or healthy subject, i.e. a subject who does not suffer from a cancer, especially a prostate cancer. The reference amount can be an average of the amounts obtained with different normal samples from different subjects.

The amounts of antisense lncRNAs obtained with subjects may also be normalized by using the amounts obtained with other RNAs which are known to have stable expression.

In a further embodiment of the above mentioned methods, the method further comprises the step of determining whether the amount of an antisense lncRNA is dysregulated, on the basis of the comparison between the amount in the biological sample and the reference amount, a higher amount of the antisense lncRNA being indicative of a higher susceptibility to have or to develop a prostate cancer.

The amount of an antisense lncRNA in a biological sample is considered as significantly different (i.e. dysregulated) compared to the reference amount, if, optionally after normalization, differences are in the order of 2-fold higher than the reference amount or more. Preferably, the amount of an antisense lncRNA in a biological sample is considered as dysregulated if the amount is at least 2.5-fold higher, or 3, 3.5, 4, 4.5 or 5-fold higher than the reference amount.

In a preferred embodiment, the present invention provides a method for detecting prostate cancer in a subject from a sample comprising the steps of: (i) determining the amount of at least one antisense lncRNA or a fragment thereof in a biological sample from the subject; (ii) comparing the amount in the sample to a reference amount derived from the amount of said antisense lncRNA in samples obtained from subjects who haven't a prostate cancer; and (iii) identifying the subject as having a prostate cancer or as having an increased risk to develop a prostate cancer when the amount of said antisense lncRNA in the sample is greater than the reference amount and/or identifying the subject as not having a prostate cancer or as not having an increased risk to develop a prostate cancer when the amount of said antisense lncRNA in the sample is equal or less than the reference amount.

Optionally, the method may further comprise performing a biopsy of the prostate of the subject to confirm that the subject has a prostate cancer when the amount of said antisense lncRNA in the sample is greater than the reference amount. Alternatively, the method may further comprise observing a biopsy of the prostate of the subject to confirm that the subject has a prostate cancer when the amount of said antisense lncRNA in the sample is greater than the reference amount.

In yet another aspect, the present method further comprises a step of treating the patient diagnosed as having a prostate cancer.

The treatment of the patient diagnosed as having a prostate cancer may comprise the administration to the patient of an effective amount of a therapeutic agent and/or the prostate resection.

The therapeutic agent may be selected from the group consisting of radiotherapeutic agents, hormonal therapy agents, chemotherapy agents, immunotherapy agents and monoclonal antibody therapy agents, preferably hormonal therapy agents.

It is understood that the administered dose of the therapeutic agent may be adapted by those skilled in the art according to the patient, the pathology, the mode of administration, etc. The dosage and regimen depends in particular on the stage and severity of the prostate cancer, the weight and general state of health of the patient and the judgment of the prescribing physician.

In some embodiments, the treatment includes one or more of open prostatectomy, minimally invasive laparoscopic robotic surgery, intensity modulated radiation therapy (IMRT), proton therapy, brachytherapy, cryotherapy, molecular-targeted therapy, vaccine therapy and gene therapy, hormone therapy, active surveillance, or a combination thereof.

In a particular embodiment, the present invention provides a method to assess the efficiency of a prostate cancer treatment in a subject having a prostate cancer comprising the steps of: (i) administering a prostate cancer treatment, i.e. an effective amount of a therapeutic agent, to the subject, (ii) determining the amount of at least one antisense lncRNA or a fragment thereof in a biological sample from the subject, (iii) comparing the amount in the sample to a reference amount derived from the amount of said antisense lncRNA in samples obtained from subjects that do not have a prostate cancer, and (iv) identifying the method as efficient or the subject as having recovered from prostate cancer when the amount of said antisense lncRNA in the sample is equal or less than the reference amount.

Optionally or alternatively, the amount at least one antisense lncRNA determined in a biological sample obtained after administration of an effective amount of a therapeutic agent may further be compare to the amount of said antisense lncRNA in a sample from the same patient obtained before the treatment, a significant decrease in the amount of said antisense lncRNA being indicative of the efficiency of said prostate cancer treatment.

### Additional Markers

In another embodiment of the above mentioned method, the method further comprises the determination of the amount of additional prostate cancer markers or fragments thereof, preferably antisense lncRNAs which are prostate cancer markers, more preferably the PCA3 antisense lncRNA.

Non-limiting other examples of prostate cancer markers include PSA and other kallikrein family members, GSTP1 (Glutathione S-Transferase Pi-1), AMACR (Alpha-Methyl-Acyl-CoA Racemase), ERG (ETS-Related Gene), gene fusions involving ETS (Erythroblast Transformation-Specific)-related genes, and PCGEM1 (prostate-specific transcript 1 (non-protein coding)).

The determination of the amounts of antisense lncRNAs of the invention and other prostate cancer markers can be carried out in the same or in different reaction mixtures, simultaneously or not.

Different prostate cancer scores can also be calculated to contribute to the diagnostic of the subject: the prostate specific antigen (PSA) Score, the Myriad Prolaris Assay (MPA) Score, the Oncotype DX Genomic Prostate Score (GPS), the Cancer of the Prostate Risk Assessment (CAPRA) Score, and the Gleason Score.

### Kit and Use of a Kit

The invention also concerns a kit for the diagnosis of prostate cancer in a subject, wherein the kit comprises probes and/or primers capable to specifically hybridize to at least one antisense lncRNA selected from the group consisting of asFBXL7, asABCC4, and asPDLIM5.

Preferably, the kit comprises probes and/or primers capable to specifically hybridize to at least two antisense lncRNA, said at least two antisense lncRNA comprising two of the antisense lncRNA described above. For instance, the kit may comprise probes and/or primers capable to specifically hybridize to asFBXL7 and asABCC4, or asFBXL7 and asPDLIM5, or asABCC4, and asPDLIM5.

Even more preferably, the kit comprises probes and/or primers capable to specifically hybridize to at least three antisense lncRNA, said at least three antisense lncRNA comprising three of the antisense lncRNA described above.

Optionally, the above mentioned kit further comprises a leaflet providing guidelines to use such a kit.

In an embodiment of the above mentioned kit, the kit further comprises probes and/or primers for the detection of additional prostate cancer markers, preferably probes and/or primers for the detection of antisense lncRNAs prostate cancer markers, more preferably probes and/or primers for the detection of the PCA3 antisense lncRNA.

Probes and/or primers of the kit, as used herein, are as defined above in any embodiment.

Preferably, for each antisense lncRNA, the kit comprises two probes, preferably Nanostring probes consisting of:
A capture-probe which comprises a nucleotide sequence hybridizing a first part of the antisense lncRNA and a molecule able to bind a solid support; and
A reporter-probe which comprises a nucleotide sequence hybridizing a second part of the antisense lncRNA and a detectable label.

Alternatively, for each antisense lncRNA, the kit comprises two probes, preferably Nanostring probes consisting of:
A capture-probe which hybridizes the antisense lncRNA and links a molecule that allows to immobilize the antisense lncRNA onto a solid support; and
A reporter-probe which hybridizes the antisense lncRNA and links a label that allows the detection of the immobilized antisense lncRNA.

In a particular embodiment, the kit comprises at least one pair of probes selected from the group consisting of:
the probes of SEQ ID No.22 and SEQ ID No.23, wherein said probes are complementary to the antisense lncRNA of SEQ ID No.1; and
the probes of SEQ ID No.24 and SEQ ID No.25, wherein said probes are complementary to the antisense lncRNA of SEQ ID No.2; and
the probes of SEQ ID No.26 and SEQ ID No.27, wherein said probes are complementary to the antisense lncRNA of SEQ ID No.3;

Preferably, the kit comprises at least two pairs of probes, said at least two pairs of probes comprising two pairs of probes selected from the group consisting of the pair of probes of SEQ ID No.22 and SEQ ID No.23 and the pair of probes consisted of SEQ ID No.24 and SEQ ID No.25, or the pair of probes of SEQ ID No.22 and SEQ ID No.23 and the pair of probes consisted of SEQ ID No.26 and SEQ ID No.27, or the pair of probes of SEQ ID No.24 and SEQ ID No.25 and the pair of probes consisted of SEQ ID No.26 and SEQ ID No.27.

More preferably, the kit comprise at least the three pairs of probes, said at least the three pairs of probes comprising described above.

In another particular embodiment, the kit comprises at least one pair of probes selected from the group consisting of:
the probes of SEQ ID No.22 and SEQ ID No.23, wherein said probes are complementary to the antisense lncRNA of SEQ ID No.1, and wherein the probe of SEQ ID No.22 further comprises an immobilizing agent and the probe of SEQ ID No. 23 further comprises a label; and
the probes of SEQ ID No.24 and SEQ ID No.25, wherein said probes are complementary to the antisense lncRNA of SEQ ID No.2, and wherein the probe of SEQ ID No.24 further comprises an immobilizing agent and the probe of SEQ ID No. 25 further comprises a label; and
the probes of SEQ ID No.26 and SEQ ID No.27, wherein said probes are complementary to the antisense lncRNA of SEQ ID No.3, and wherein the probe of SEQ ID No.26 further comprises an immobilizing agent and the probe of SEQ ID No. 27 further comprises a label.

Preferably, the immobilizing agent comprises biotin.

Preferably, the label is a fluorescent label, more preferably a combination of several fluorophores, even more preferably a combination of six fluorophores selected from four different fluorophores.

Preferably, the kit comprises at least two pairs of probes selected from the pairs of probes above mentioned. More preferably, the kit comprises at least the three pairs of probes above mentioned.

When several pairs of probes are included in the kit, the label is different for each of the probes presenting a label.

Alternatively, for each antisense lncRNA, the kit comprises:
at least one primer pair;
optionally, RT primers, preferably random primers, alternatively a RT primer complementary to said at least one antisense lncRNA.
optionally, a labeled probe.

Preferably, the labeled probe, is a fluorescent probe, more preferably a dual labeled probe which is complementary to a sequence of the target sequence localized between the two primers, even more preferably, the dual label probe is aimed to fluoresce upon 5′→3′ exonuclease activity.

Preferably, the at least one primer pair consists in primers that are complementary to either exon or intron sequences of the pre-antisense lncRNA, and are obligatory out of exons of the corresponding pre-mRNA. More preferably, the primers are complementary to exon sequences of the pre-antisense lncRNA, and are obligatory out of exons of the corresponding pre-mRNA.

In a particular embodiment, the kit comprises at least one primer pair selected from the group consisting of:
the primers of SEQ ID No.4 and SEQ ID No.5, wherein said primers are specific of the cDNA reverse transcript from asABCC4; and
the primers of SEQ ID No.6 and SEQ ID No.7, wherein said primers are specific of the cDNA reverse transcript from asABCC4; and
the primers of SEQ ID No.8 and SEQ ID No.9, wherein said primers are specific of the cDNA reverse transcript from asPDLIM5; and
the primers of SEQ ID No.10 and SEQ ID No.11, wherein said primers are specific of the cDNA reverse transcript from asPDLIM5; and
the primers of SEQ ID No.12 and SEQ ID No.13, wherein said primers are specific of the cDNA reverse transcript from asPDLIM5; and
the primers of SEQ ID No.14 and SEQ ID No.15, wherein said primers are specific of the cDNA reverse transcript from asPDLIM5; and
the primers of SEQ ID No.16 and SEQ ID No.17, wherein said primers are specific of the cDNA reverse transcript from asFXL7; and
the primers of SEQ ID No.18 and SEQ ID No.19, wherein said primers are specific of the cDNA reverse transcript from asFXL7; and
the primers of SEQ ID No.20 and SEQ ID No.21, wherein said primers are specific of the cDNA reverse transcript from asFXL7.

Preferably, the kit comprises a combination of two different primer pairs specific of the same antisense lncRNA. For instance, this combination of two primer pairs can be selected from the group consisting of: the primer pair of SEQ ID No.4 and SEQ ID No.5 and the primer pair of SEQ ID No.6 and SEQ ID No.7, the primer pair of SEQ ID No.8 and SEQ ID No.9 and the primer pair of SEQ ID No.10 and SEQ ID No.11, the primer pair of SEQ ID No.8 and SEQ ID No.9 and the primer pair of SEQ ID No.12 and SEQ ID No.13; the primer pair of SEQ ID No.8 and SEQ ID No.9 and the primer pair of SEQ ID No.14 and SEQ ID No.15, the primer pair of SEQ ID No.10 and SEQ ID No.11 and the primer pair of SEQ ID No.12 and SEQ ID No.13, the primer pair of SEQ ID No.10 and SEQ ID No.11 and the primer pair of SEQ ID No.14 and SEQ ID No.15, the primer pair of SEQ ID No.12 and SEQ ID No.13 and the primer pair of SEQ ID No.14 and SEQ ID No.15, the primer pair of SEQ ID No.16 and SEQ ID No.17 and the primer pair of SEQ ID No.18 and SEQ ID No.19, the primer pair of SEQ ID No.15 and SEQ ID No.16 and the primer pair of SEQ ID No.20 and SEQ ID No.21, and the primer pair of SEQ ID No.18 and SEQ ID No.19 and the primer pair of SEQ ID No.20 and SEQ ID No.21.

In a preferred embodiment of the above mentioned kit, the kit comprises at least two primer pairs targeting two different antisense lncRNA and selected from the group consisting of: the primer pair of SEQ ID No.4 and SEQ ID No.5 and the primer pair of SEQ ID No.8 and SEQ ID No.9, the primer pair of SEQ ID No.4 and SEQ ID No.5 and the primer pair of SEQ ID No.10 and SEQ ID No.11, the primer pair of SEQ ID No.4 and SEQ ID No.5 and the primer pair of SEQ ID No.12 and SEQ ID No.13, the primer pair of SEQ ID No.4 and SEQ ID No.5 and the primer pair of SEQ ID No.14 and SEQ ID No.15, the primer pair of SEQ ID No.4 and SEQ ID No.5 and the primer pair of SEQ ID No.16 and SEQ ID No.17, the primer pair of SEQ ID No.4 and SEQ ID No.5 and the primer pair of SEQ ID No.18 and SEQ ID No.19, the primer pair of SEQ ID No.4 and SEQ ID No.5 and the primer pair of SEQ ID No.20 and SEQ ID No.21, the primer pair of SEQ ID No.6 and SEQ ID No.7 and the primer pair of SEQ ID No.8 and SEQ ID No.9, the primer pair of SEQ ID No.6 and SEQ ID No.7 and the primer pair of SEQ ID No.10 and SEQ ID No.11, the primer pair of SEQ ID No.6 and SEQ ID No.7 and the primer pair of SEQ ID No.12 and SEQ ID No.13, the primer pair of SEQ ID No.6 and SEQ ID No.7 and the primer pair of SEQ ID No.14 and SEQ ID No.15, the primer pair of SEQ ID No.6 and SEQ ID No.7 and the primer pair of SEQ ID No.16 and SEQ ID No.17, the primer pair of SEQ ID No.6 and SEQ ID No.7 and the primer pair of SEQ ID No.18 and SEQ ID No.19, the primer pair of SEQ ID No.6 and SEQ ID No.7 and the primer pair of SEQ ID No.20 and SEQ ID No.21, the primer pair of SEQ ID No.8 and SEQ ID No.9 and the primer pair of SEQ ID No.16 and SEQ ID No.17, the primer pair of SEQ ID No.8 and SEQ ID No.9 and the primer pair of SEQ ID No.18 and SEQ ID No.19, the primer pair of SEQ ID No.8 and SEQ ID No.9 and the primer pair of SEQ ID No.20 and SEQ ID No.21, the primer pair of SEQ ID No.10 and SEQ ID No.11 and the primer pair of SEQ ID No.16 and SEQ ID No.17, the primer pair of SEQ ID No.10 and SEQ ID No.11 and the primer pair of SEQ ID No.18 and SEQ ID No.19, the primer pair of SEQ ID No.10 and SEQ ID No.11 and the primer pair of SEQ ID No.20 and SEQ ID No.21, the primer pair of SEQ ID No.12 and SEQ ID No.13 and the primer pair of SEQ ID No.16 and SEQ ID No.17, the primer pair of SEQ ID No.12 and SEQ ID No.13 and the primer pair of SEQ ID No.18 and SEQ ID No.19, the primer pair of SEQ ID No.12 and SEQ ID No.13 and the primer pair of SEQ ID No.20 and SEQ ID No.21, the primer pair of SEQ ID No.14 and SEQ ID No.15 and the primer pair of SEQ ID No.16 and SEQ ID No.17, the primer pair of SEQ ID No.14 and SEQ ID No.15 and the primer pair of SEQ ID No.18 and SEQ ID No.19, and the primer pair of SEQ ID No.14 and SEQ ID No.15 and the primer pair of SEQ ID No.20 and SEQ ID No.21.

Preferably, the kit comprises a combination of four different primer pairs specific of two different antisense lncRNAs and selected from the group consisting of: the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.1, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15, the primer pair of SEQ ID No.4 and SEQ ID No.5 and the primer pair of SEQ ID No.6 and SEQ ID No.7 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.16 and SEQ ID No.7 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.10 and SEQ ID No.11 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.8 and SEQ ID No.9 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.10 and SEQ ID No.11 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.10 and SEQ ID No.11 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.20 and SEQ ID No.21, and the primer pair of SEQ ID No.12 and SEQ ID No.13 and the primer pair of SEQ ID No.14 and SEQ ID No.15 and the primer pair of SEQ ID No.18 and SEQ ID No.19 and the primer pair of SEQ ID No.20 and SEQ ID No.21.

In an even more preferred embodiment of the above mentioned kit, the kit comprises at least three primer pairs targeting three different antisense lncRNA and selected from the group consisting of: the primer pair of SEQ ID No.4 and SEQ ID No.5 and the primer pair of SEQ ID No.8 and SEQ ID No.9 and the primer pair of SEQ ID No.16 and SEQ ID No.17, the primer pair of SEQ ID No.4 and SEQ ID No.5 and the primer pair of SEQ ID No.8 and SEQ ID No.9 and the primer pair of SEQ ID No.18 and SEQ ID No.19, the primer pair of SEQ ID No.4 and SEQ ID No.5 and the primer pair of SEQ ID No.8 and SEQ ID No.9 and the primer pair of SEQ ID No.20 and SEQ ID No.21, the primer pair of SEQ ID No.4 and SEQ ID No.5 and the primer pair of SEQ ID No.10 and SEQ ID No.11 and the primer pair of SEQ ID No.16 and SEQ ID No.17, the primer pair of SEQ ID No.4 and SEQ ID No.5 and the primer pair of SEQ ID No.10 and SEQ ID No.11 and the primer pair of SEQ ID No.18 and SEQ ID No.19, the primer pair of SEQ ID No.4 and SEQ ID No.5 and the primer pair of SEQ ID No.10 and SEQ ID No.11 and the primer pair of SEQ ID No.20 and SEQ ID No.21, the primer pair of SEQ ID No.4 and SEQ ID No.5 and the primer pair of SEQ ID No.12 and SEQ ID No.13 and the primer pair of SEQ ID No.16 and SEQ ID No.17, the primer pair of SEQ ID No.4 and SEQ ID No.5 and the primer pair of SEQ ID No.12 and SEQ ID No.13 and the primer pair of SEQ ID No.18 and SEQ ID No.19, the primer pair of SEQ ID No.4 and SEQ ID No.5 and the primer pair of SEQ ID No.11 and SEQ ID No.12 and the primer pair of SEQ ID No.20 and SEQ ID No.21, the primer pair of SEQ ID No.4 and SEQ ID No.5 and the primer pair of SEQ ID No.14 and SEQ ID No.15 and the primer pair of SEQ ID No.16 and SEQ ID No.17, the primer pair of SEQ ID No.4 and SEQ ID No.5 and the primer pair of SEQ ID No.14 and SEQ ID No.15 and the primer pair of SEQ ID No.18 and SEQ ID No.19, the primer pair of SEQ ID No.4 and SEQ ID No.5 and the primer pair of SEQ ID No.14 and SEQ ID No.15 and the primer pair of SEQ ID No.20 and SEQ ID No.21, the primer pair of SEQ ID No.6 and SEQ ID No.7 and the primer pair of SEQ ID No.8 and SEQ ID No.9 and the primer pair of SEQ ID No.16 and SEQ ID No.17, the primer pair of SEQ ID No.6 and SEQ ID No.7 and the primer pair of SEQ ID No.8 and SEQ ID No.9 and the primer pair of SEQ ID No.18 and SEQ ID No.19, the primer pair of SEQ ID No.6 and SEQ ID No.7 and the primer pair of SEQ ID No.8 and SEQ ID No.9 and the primer pair of SEQ ID No.20 and SEQ ID No.21, the primer pair of SEQ ID No.6 and SEQ ID No.7 and the primer pair of SEQ ID No.10 and SEQ ID No.11 and the primer pair of SEQ ID No.16 and SEQ ID No.17, the primer pair of SEQ ID No.6 and SEQ ID No.7 and the primer pair of SEQ ID No.10 and SEQ ID No.11 and the primer pair of SEQ ID No.18 and SEQ ID No.19, the primer pair of SEQ ID No.6 and SEQ ID No.7 and the primer pair of SEQ ID No.10 and SEQ ID No.11 and the primer pair of SEQ ID No.20 and SEQ ID No.21, the primer pair of SEQ ID No.6 and SEQ ID No.7 and the primer pair of SEQ ID No.12 and SEQ ID No.13 and the primer pair of SEQ ID No.16 and SEQ ID No.17, the primer pair of SEQ ID No.6 and SEQ ID No.7 and the primer pair of SEQ ID No.12 and SEQ ID No.13 and the primer pair of SEQ ID No.18 and SEQ ID No.19, the primer pair of SEQ ID No.6 and SEQ ID No.7 and the primer pair of SEQ ID No.11 and SEQ ID No.12 and the primer pair of SEQ ID No.20 and SEQ ID No.21, the primer pair of SEQ ID No.6 and SEQ ID No.7 and the primer pair of SEQ ID No.14 and SEQ ID No.15 and the primer pair of SEQ ID No.16 and SEQ ID No.17, the primer pair of SEQ ID No.6 and SEQ ID No.7 and the primer pair of SEQ ID No.14 and SEQ ID No.15 and the primer pair of SEQ ID No.18 and SEQ ID No.19, and the primer pair of SEQ ID No.6 and SEQ ID No.7 and the primer pair of SEQ ID No.14 and SEQ ID No.15 and the primer pair of SEQ ID No.20 and SEQ ID No.21.

Preferably, the kit comprises a combination of six different primer pairs specific of three different antisense lncRNAs selected from the group consisting of: the primer pairs of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.10 and SEQ ID No.11 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.8 and SEQ ID No.9 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.12 and SEQ ID No.13 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.16 and SEQ ID No.17 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.10 and SEQ ID No.11 and the primer pair of SEQ ID No.14 and SEQ ID No.15 and the primer pair of SEQ ID No.18 and SEQ ID No.19 and the primer pair of SEQ ID No.20 and SEQ ID No.21, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.18 and SEQ ID No.19, the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.16 and SEQ ID No.17 and the primer pair of SEQ ID No.20 and SEQ ID No.21, and the primer pair of SEQ ID No.4 and SEQ ID No.5 and the primer pair of SEQ ID No.6 and SEQ ID No.7 and the primer pair of SEQ ID No.12 and SEQ ID No.13 and the primer pair of SEQ ID No.14 and SEQ ID No.15 and the primer pair of SEQ ID No.18 and SEQ ID No.19 and the primer pair of SEQ ID No.20 and SEQ ID No.21.

The above mentioned combination may further comprise additional primer pairs, preferably primer pairs targeting other antisense lncRNA which are diagnosis marker for prostate cancer, more preferably primer pairs for PCA3 quantitative amplification.

The invention also concerns the use of a kit as described above in the diagnosis of prostate cancer in a subject.

Preferably, the subject is an animal, more preferably a mammal, even more preferably a human.

In a most preferred embodiment, the subject is an adult man of at least 50 years old.

Further aspects and advantages of the present invention will be described in the following examples, which should be regarded as illustrative and not limiting.

### EXAMPLES

Material and Methods

### Prostate and Breast Tissues, RNA Samples:

Prostate tissues were provided by the Mondor Hospital and breast tissues by Institut Curie. The histological status of each tumor was determined by respective clinical pathology services.

Total RNA was extracted from tissues or urines using Qiagen kit, quantified and quality controlled using a 2100 Bioanalyzer (Agilent). RNA samples with RNA Integrity Number (RIN) above 6 were depleted for ribosomal RNA and converted into cDNA library using a TruSeq Stranded Total Library Preparation kit (Illumina). cDNA libraries were normalized using an Illumina Duplex-specific Nuclease (DSN) protocol.

### RNA Sequencing and Data Analysis:

Strand-specific cDNA library were sequenced in a paired-end mode on HiSeg™ 2500 (Illumina, NGS Curie platform). At least a 20× coverage per sample was considered as minimum of unique sequences for further data analysis. RNAseq data has been further analysed by in-house developed computational pipeline using a novel extended antisense annotation file to isolate all antisense lncRNA expressed to 0.8 RPKM.

### Signature of Prostate Tumor Tissues:

Differential expression analysis between prostate normal and tumor samples was performed using the DESeq R package. Only antisense lncRNAs with an expression fold change >2 and adjusted P-value below 0.05 were retained as differentially expressed to constitute prostate tumor signature.

### Receiver Operating Curve (ROC) Analysis:

ROC curves assessed the assignment of 89 patients as high-risk versus low or intermediate risk, based on PCGEM1 only, PCA3 only or the sum of 3 novel antisense lncRNA (asFBXL7, asABCC4 and asPDLIM5) or the sum of 4 lncRNAs (asFBXL7, asABCC4, asPDLIM5 and PCA3), using pROC R-package and log2(expression) scores from Nanostring data.

Reverse transcription and quantitative PCR: Reverse Transcription was performed on 100 ng of total RNA using gene-specific, strand-specific or random primers and the SuperScript II (Invitrogen) reverse transcriptase. cDNA for each transcript was quantified relative to the POLR2A housekeeping gene by the real-time PCR with LightCycler-time PCR with as quantified relative to Roche LightCycler480. Error bars represent standard deviations of three independent RT.

NanoString nCounter Gene Expression Assay:

Total RNA isolated from 80 tumors and 9 normal prostate tissues was used for direct digital detection of 13 different RNAs: 3 housekeeping genes (RPL11, GAPDH and POLR2A), 4 mRNAs deregulated in our or published RNAseq datasets (HPN, OR51E2, PDLIMS, PCA3 and SLC14A1), 3 lncRNAs previously reported as deregulated in different cancers (HOTAIR, ANRIL and PCGEM1), and 3 antisense lncRNA (asFBXL7, asPDLIM5 and asABCC4). Each target gene of interest was detected using reporter and capture probes carrying 35- to 50-base target-specific sequences on Nanostring nCounter V2 (Curie Genomic platform).

Results

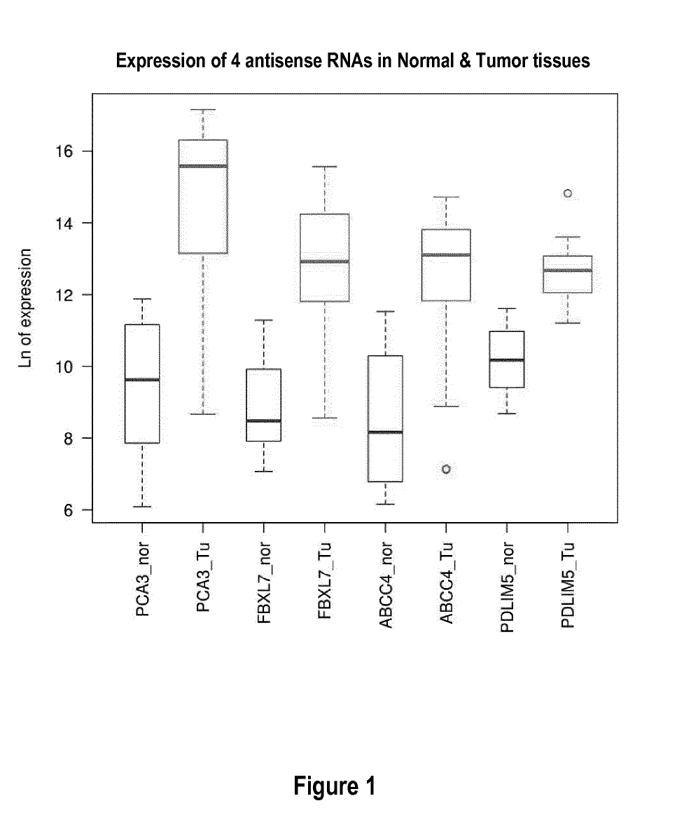

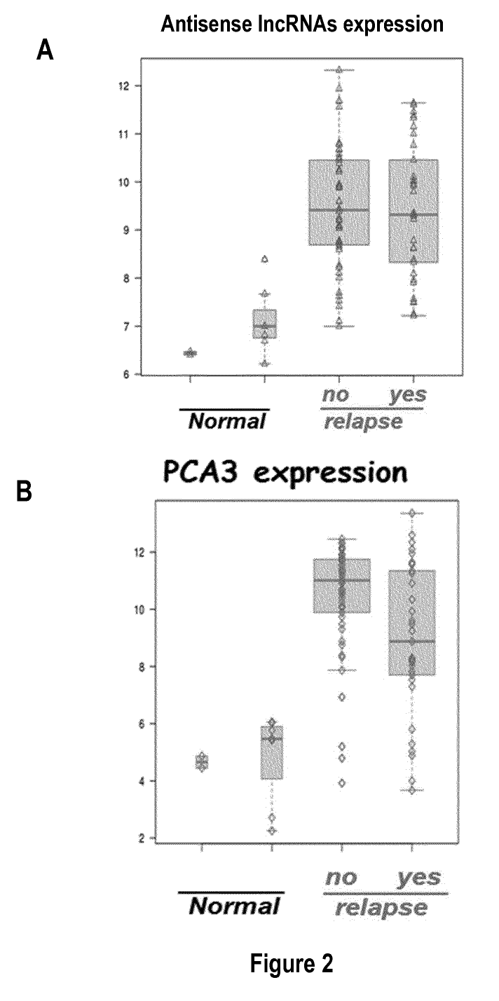

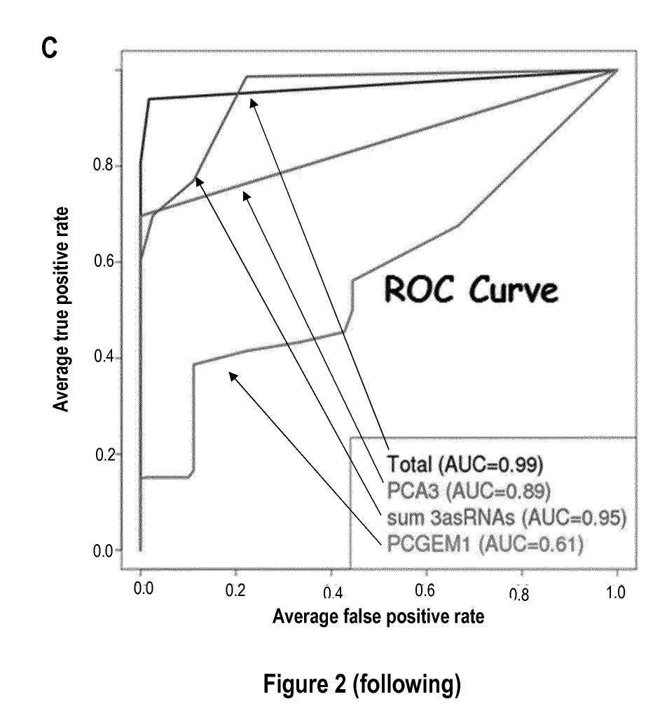

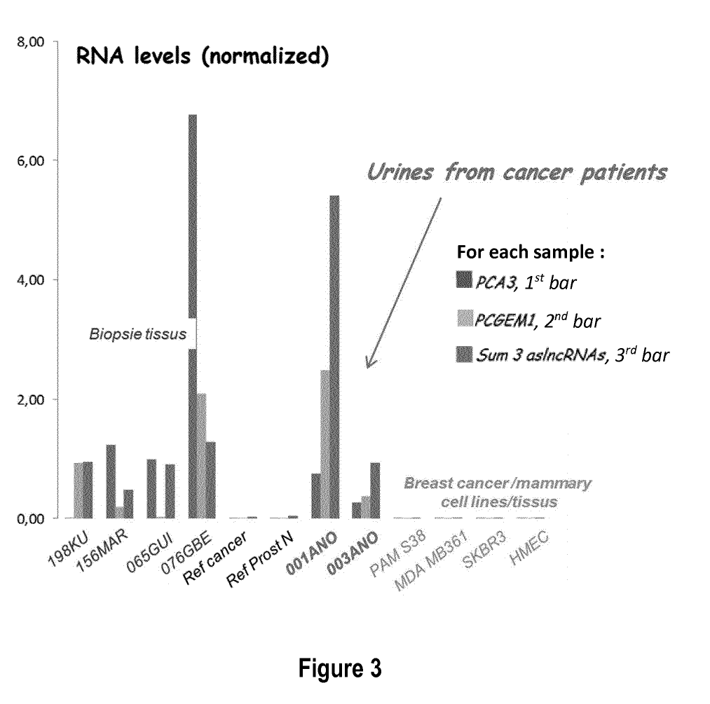

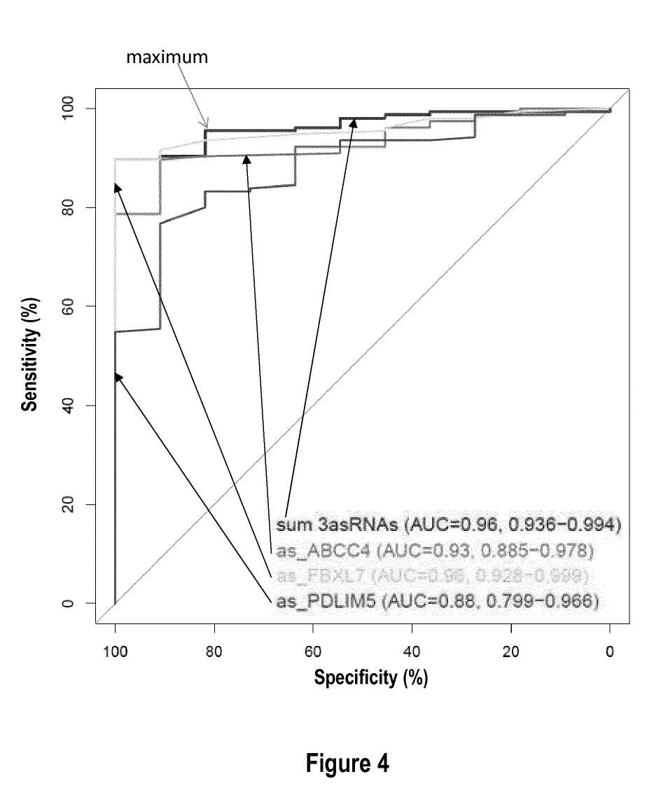

Here, we investigated the presence of antisense lncRNAs by performing RNA-seq experiments within 8 normal and 16 tumor prostate tissues. Differential expression analysis revealed 157 antisense lncRNAs significantly overexpressed in tumors encompassing 48 previously annotated antisense lncRNA, and 109 novel transcripts. Interestingly, the 3 most up-regulated antisense lncRNA showed similar fold-changes as the classic prostate cancer marker PCA3 albeit with lower dispersion, revealing a novel robust restricted signature for prostate cancer (FIG. 1). Expression of these three antisense lncRNAs have been further validated by the quantitative RT-PCR (data not shown) and nanostring single molecule quantification, all of them being significantly (at least 5 fold) higher expressed in tumor tissues comparing to normal ones (FIG. 2A). Interestingly, antisense lncRNA nanostring signals were constant in relapse and non-relapse tumor patients revealing robust detection accuracy. In contrast, the commercial PCA3 RNA level failed to maintain such robustness since the relapse patients show a dramatic decrease in PCA3 expression measurements (FIG. 2B). Strikingly, a prediction analysis on a cohort of 89 patients from RNA extraction measured by Nanostring demonstrated that the “restricted signature” discriminates relapse patients more efficiently than PCA3 alone (AUC 0.95 and 0.89, respectively; FIG. 2C). In contrast, these novel markers had similar or lower AUC than PCA3 for non-relapsing cases (data not shown), supporting their combined usage as markers for prognostic studies. Strikingly, the detection of prostate tumors is compatible with RNA extracted directly from urines after prostate massages (FIG. 3), suggesting an easy, fast and cost effective way to detect prostate tumors at early stages without intrusive biopsy.

The inventors have discovered three new antisense lncRNA that allow discriminating prostate tumors from normal prostate tissues, but also prostate tumors from breast tumor and normal tissues. Interestingly, using a cohort of 89 patients' biopsies they showed comparable fold-changes of expression as PCA3 albeit with lower dispersion, revealing a novel robust restricted signature for prostate cancer. Strikingly, a prediction analysis on urines patients based on NanoString antisense lncRNAs measurements, demonstrated that the restricted signature discriminates prostate tumors more efficiently than PCA3.

The inventors also compared sensitivity and specificity prediction for the three new antisense lncRNA alone and in combination. ROC curves analysis show that each antisense lncRNA has a significant prediction level: area under the curve are of 0.88 for asPDLIM5, 0.96 for asFBXL7, and 0.93 for asABCC4. The sum of the three antisense lncRNA is characterized by an area under the curve of 0.96, indicating an excellent robustness of detection with a maximum of both specificity and sensitivity when compared to the 3 separately.

1-14. (canceled)

15. An in vitro method for prostate cancer diagnosis of a subject, wherein the method comprises the step of determining the amount of at least one antisense lncRNA or a fragment thereof of at least 50 nucleotides in a biological sample from said subject, said at least one antisense lncRNA being selected from the group consisting of asFBXL7, asABCC4 and asPDLIM5, and wherein an increased amount of the at least one antisense lncRNA is indicative of prostate cancer.

16. The method according to claim 15, wherein the method comprises the determination of the amounts of at least three antisense lncRNAs and wherein said at least three antisense lncRNAs comprise asFBXL7, asABCC4 and asPDLIM5.

17. The method according to claim 15, wherein the method further comprises the determination of the amounts of additional prostate cancer markers or fragments thereof.

18. The method according to claim 15, wherein said sample is a body fluid.

19. The method according to claim 15, wherein the antisense lncRNA amount is determined by amplification or by hybridization.

20. The method according to claim 15, wherein the subject is a mammal.

21. The method according to claim 20, wherein the mammal is a man.

22. The method according to claim 17, wherein the additional prostate cancer markers or fragments thereof are antisense lncRNAs prostate cancer markers or fragments thereof.

23. The method according to claim 22, wherein the antisense lncRNA prostate cancer marker or fragment thereof is PCA3 antisense lncRNA or a fragment thereof.

24. The method according to claim 18, wherein the bodily fluid is urine.

25. The method according to claim 19, wherein the amount of antisense lncRNA is determined by quantitative RT-PCR or the Nanostring method.

26. The method according to claim 21, wherein the man is at least 50 years old.

27. A kit for the diagnosis of prostate cancer in a subject, wherein the kit comprises (i) probes and/or primers capable to specifically hybridize to asFBXL7; and/or (ii) probes and/or primers capable to specifically hybridize to asABCC4; and/or (iii) probes and/or primers capable to specifically hybridize to asPDLIM5; and optionally, a leaflet providing guidelines to use such a kit.

28. The kit according to claim 27, wherein the kit further comprises probes and/or primers for the detection of additional prostate cancer markers.

29. The kit according to claim 27, wherein, for each antisense lncRNA, the kit comprises two probes:
a capture-probe which comprises a nucleotide sequence hybridizing a first part of the antisense lncRNA and a molecule able to bind a solid support; and
a reporter-probe which comprises a nucleotide sequence hybridizing a second part of the antisense lncRNA and a detectable label.

30. The kit according to claim 28, wherein the probes and/or primers for the detection of additional prostate cancer markers are probes and/or primers for the detection of antisense lncRNAs prostate cancer markers.

31. The kit according to claim 30, wherein the probes and/or primers for the detection of antisense lncRNAs prostate cancer markers are for the detection of the PCA3 antisense lncRNA.
